(19) United States
(12) Patent Application Publication
(10) Pub. No.: US 2011/0269734 A1
(43) Pub. Date: Nov. 3, 2011

(54) PIPERIDINYL GPCR AGONISTS

(75) Inventors: Lisa Sarah Bertram, Oxford (GB); Matthew Colin Thor Fyfe, Oxford (GB); Revathy Perpetua Jeevaratnam, Oxford (GB); John Keily, Oxford (GB); Thomas Martin Krulle, Oxford (GB); Chrystelle Marie Rasamison, Oxford (GB); Colin Peter Sambrook-Smith, Oxford (GB); Simon Andrew Swain, Oxford (GB)

(73) Assignee: Prosidion Limited, Oxford (GB)

(21) Appl. No.: 13/003,546

(22) PCT Filed: Jul. 10, 2009
(86) PCT No.: PCT/GB2009/050825

§ 371 Date: Apr. 28, 2011

(30) Foreign Application Priority Data
Jul. 10, 2008 (GB) 0812622.9
Jul. 10, 2008 (GB) 0812646.8
Feb. 13, 2009 (GB) 0902391.2

(51) Int. Cl. A61K 31/397; A61K 31/506; C07D 401/14; A61P 9/12; A61K 31/4545; A61P 3/04; A61P 3/10; A61P 3/06; C07D 401/04; A61K 31/501

(52) U.S. Cl. 514/210.2; 544/332; 514/275; 544/238; 514/252.03; 546/194; 514/318

(57) ABSTRACT

Compounds of formula (I):or pharmaceutically acceptable salts thereof, are GPCR agonists and are useful as for the treatment of diabetes and obesity.

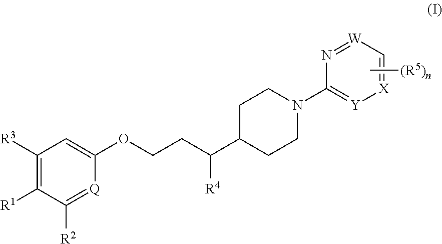

### BACKGROUND OF THE INVENTION

The present invention is directed to G-protein coupled receptor (GPCR) agonists. In particular, the present invention is directed to agonists of GPR119 that are useful for the treatment of obesity, e.g. as regulators of satiety, metabolic syndrome and for the treatment of diabetes.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) places people at high risk of coronary artery disease, and is characterized by a cluster of risk factors including central obesity (excessive fat tissue in the abdominal region), glucose intolerance, high triglycerides and low HDL cholesterol, and high blood pressure. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of obesity and diabetes.

International patent applications WO2005/061489, WO2006/070208 and WO2006/067532 disclose heterocyclic derivatives as GPR119 receptor agonists. International patent applications WO2006/067531, WO2007/003960, WO2007/003961, WO2007/003962 and WO2007/003964, WO2007/116230 and WO2007/116229 disclose GPR119 receptor agonists.

The present invention relates to agonists of GPR119 which are useful for the treatment of diabetes and as peripheral regulators of satiety, e.g. for the treatment of obesity and metabolic syndrome.

### SUMMARY OF THE INVENTION

Compounds of formula (I):

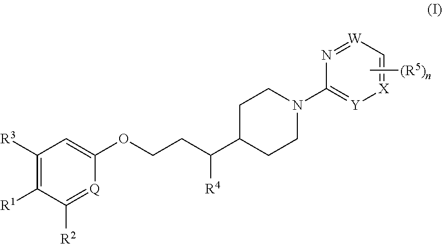

or pharmaceutically acceptable salts thereof, are agonists of GPR119 and are useful for the prophylactic or therapeutic treatment of diabetes and obesity.

### DETAILED DESCRIPTION OF THE INVENTION

The present invention is directed to a compound of formula (I), or a pharmaceutically acceptable salt thereof:

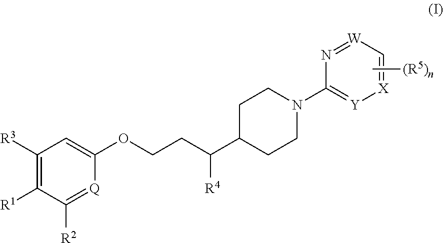

wherein Q is CH or N;

one of W, X and Y is N or CH and the others are CH where the H may be replaced by R⁵ when present;

R¹ is —SO₂Me or —CONHR⁶;

R², R³ and R⁴ are independently selected from hydrogen and methyl;

n is 0, 1 or 2;

R⁵ is independently C₁₋₄ alkyl, C₁₋₄ alkoxy, fluoro, chloro, C₁₋₃ fluoroalkyl or benzyl;

R⁶ is hydrogen, 3-azetidinyl, 3-pyrrolidinyl, 3-piperidinyl or 4-piperidinyl, wherein the azetidinyl, pyrrolidinyl and piperidinyl rings may be optionally substituted with OH, CH₂OH or CH₃; C₁₋₃ alkyl, C₂₋₄ alkyl substituted by —N(R⁷)₂ and/or one or two hydroxy groups, or C₁₋₄ alkyl substituted by a 4- to 6-membered nitrogen-containing heterocyclic ring; and

R⁷ is independently hydrogen or methyl.

In one embodiment of the invention, Q is CH. In another Q is N.

Q is preferably CH.

In one embodiment of the invention, R¹ is —SO₂Me. In another, R¹ is —CONHR⁶.

R¹ is preferably —CONHR⁶.

One or both of R² and R³ are preferably methyl, for example R² is methyl and R³ is hydrogen.

In one embodiment of the invention R⁴ is hydrogen and in another R⁴ is methyl. R⁴ is preferably hydrogen. When R⁴ is methyl, the stereocenter produced preferably has the (R)-configuration.

n is preferably 1.

When n is 1, R⁵ is preferably meta or para, more preferably para, to the point of attachment to the piperidinyl nitrogen.

R⁵ is preferably C₁₋₃ alkyl, fluoro, chloro or C₁₋₃ fluoroalkyl, more preferably C₂₋₃ alkyl, chloro or C₁₋₃ fluoroalkyl, especially chloro, ethyl or isopropyl, more especially chloro.

W and X are preferably CH, for example W, X and Y may be CH. Y is preferably N.

R⁶ is preferably hydrogen or C₂₋₃ alkyl substituted by —N(R⁷)₂ or one or two hydroxy groups, for example hydrogen or C₂₋₃ alkyl substituted by one or two hydroxy groups. R⁶ is more preferably C₂₋₃ alkyl substituted by one or two hydroxy groups, e.g. 2-hydroxyethyl, 2-hydroxy-1-methylethyl, 2,3-dihydroxypropyl or 2-hydroxy-1-hydroxymethylethyl.

When R⁶ is C₂₋₄alkyl substituted a 4- to 6-membered nitrogen-containing heterocyclic ring, suitable 4- to 6-membered nitrogen-containing heterocyclic rings include pyrrolidinyl and azetidinyl.

R⁷ is preferably hydrogen.

A group of compounds which may be mentioned are those where:

Q is N;

one of W, X and Y is N or CH and the others are CH where the H may be replaced by R⁵ when present;

R¹ is —SO₂Me or —CONHR⁶;

R², R³ and R⁴ are independently selected from hydrogen and methyl;

n is 0, 1 or 2;

R⁵ is C₁₋₄ alkyl, C₁₋₄ alkoxy, fluoro, chloro, C₁₋₃ fluoroalkyl or benzyl; and

R⁶ is hydrogen, 3-azetidinyl, 3-pyrrolidinyl, C₁₋₃ alkyl, or C₂₋₃ alkyl substituted by amino or one or two hydroxy groups;

and pharmaceutically acceptable salts thereof.

A further group of compounds which may be mentioned are those where:

Q is CH;

one of W, X and Y is N or CH and the others are CH where the H may be replaced by R⁵ when present;

R¹ is —SO₂Me or —CONHR⁶;

when R¹ is —SO₂Me, R² and R³ are hydrogen, and when R¹ is —CONHR⁶, R² and R³ are independently selected from hydrogen and methyl;

R⁴ is hydrogen or methyl;

n is 0, 1 or 2;

R⁵ is independently C₁₋₄ alkyl, C₁₋₄ alkoxy, fluoro, chloro or C₁₋₃ fluoroalkyl; and

R⁶ is hydrogen, 3-azetidinyl, 3-pyrrolidinyl, 4-piperidinyl, C₁₋₃ alkyl, or C₂₋₄alkyl substituted by amino and/or one or two hydroxy groups, or by a 4- or 5-membered nitrogen-containing heterocyclic ring.

In this further group of compounds, certain compounds which may be mentioned are those where R⁶ is hydrogen, 3-azetidinyl, 3-pyrrolidinyl, C₁₋₃ alkyl, or C₂₋₃ alkyl substituted by amino or one or two hydroxy groups.

While the preferred groups for each variable have generally been listed above separately for each variable, preferred compounds of this invention include those in which several or each variable in formula (I) is selected from the preferred, more preferred or particularly listed groups for each variable. Therefore, this invention is intended to include all combinations of preferred, more preferred and particularly listed groups.

Specific compounds of the invention which may be mentioned are those included in the Examples and pharmaceutically acceptable salts thereof.

As used herein, unless stated otherwise, “alkyl” means carbon chains which may be linear or branched or combinations thereof. Examples of alkyl groups include methyl, ethyl, propyl, isopropyl, butyl, sec- and tert-butyl.

“Fluoroalkyl” means alkyl groups substituted by one or more fluoro atoms, e.g. CHF₂ and CF₃.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. Salts derived from bases include those derived from bases such as, for example, potassium and sodium salts and the like. Salts derived from pharmaceutically acceptable non-toxic acids, include those derived from inorganic and organic acids such as, for example, hydrochloric, methanesulfonic, sulfuric, p-toluenesulfonic acid and the like.

Since the compounds of formula (I) are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, wherein R¹, R², R³, R⁴, R⁵, Q, W, X and Y are defined as above, PG represents a protecting group, DG represents a displaceable group e.g, F, Cl, Br, MeSO₂, and Ak is C₁₋₂alkyl.

Unichiral building blocks for compounds of formula (I), where R⁴ is Me, can be readily prepared from known compounds (Scheme 1). For example, the ethyl ester of compound (II) where PG is Boc has been previously reported (U.S. Pat. No. 6,518,423). Saponification and hydrogenation, under standard conditions, will yield the racemic compound of formula (III). Chiral reduction of the alkenoic acid (II) under suitable conditions, such as a hydrogenation in the presence of a chiral catalyst, yields compounds of formula (III) in high enantiomeric excess. An example of a suitable catalyst is [Rh(norbornadiene)₂]BF₄ and (S)-1-[(R)-2-(di-tert-butylphosphino)ferrocenyl]ethylbis(2-methylphenyl)phosphine. Compounds of formula (IV) can then be obtained by reduction of the carboxylic acids of formula (III) under standard conditions, for example employing borane in a suitable solvent such as THF.

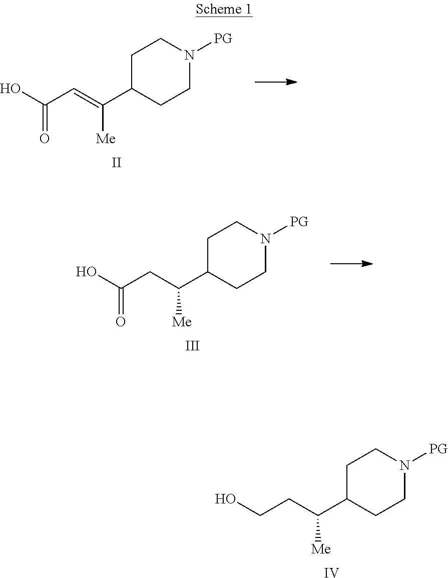

Compounds of formula (I) can be prepared as outlined in Scheme 2. Compounds of formula (VII) can be synthesized by reaction of a phenol of formula (V) with an alcohol of formula (VI) under, for example, Mitsunobu conditions. Subsequent removal of the protecting group, using conditions well known to those with skill in the art, and reaction with an appropriate 6-membered heteroaromatic halide, yields compounds of formula (I).

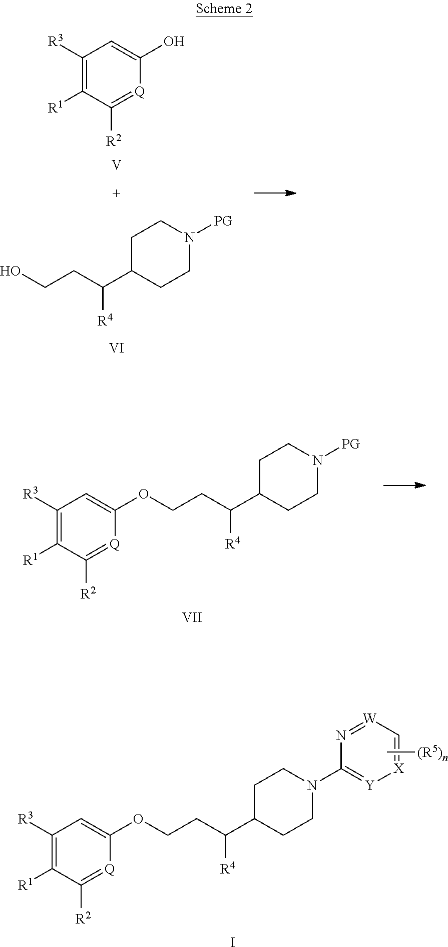

Compounds of formula (I) where Q is CH and R¹ is —SO₂Me can also be prepared from compounds of formula (VI), by reaction with the appropriate 4-methanesulfonylphenol under Mitsunobu conditions (for example using a suitable solvent such as THF, at between 0° C. and room temperature, followed by the addition of triphenylphosphine and diisopropylazo-dicarboxylate) followed by removal of the protecting group under conditions well known to those with skill in the art. Subsequent reaction with an appropriate 6-membered heteroaromatic halide in dimethylsulfoxide and 1,8-diazabicyclo[5.4.0]undec-7-ene at 100° C., yields compounds of formula (I) (Scheme 2).

Compounds of formula (I) where Q is N and R¹ is SO₂Me can also be produced as outlined in Scheme 3. For example, the compound of formula (VIII) where R² is methyl, R³ is hydrogen and DG is fluorine is commercially available. Bromine-metal exchange using butyllithium in a suitable solvent such as toluene, in the presence of a chelating agent such as TMEDA, if required, at low temperature followed by the addition of dimethyldisulfide affords the corresponding sulfide. Subsequent oxidation of the sulfide to the sulfone, using a standard oxidizing agent such as m-CPBA, affords compounds of formula (IX). Compounds of formula (X) are then formed by displacement of the leaving group in compounds of formula (IX) with the alkoxide of a compound of formula (VI) under standard conditions. Removal of the protecting group using conditions well known to those with skill in the art, followed by reaction with an appropriate 6-membered heteroaromatic halide, under suitable conditions, such as standard displacement conditions in DMSO in the presence of 1,8-diazabicyclo[5.4.0]undec-7-ene, yields compounds of formula (I) as described above.

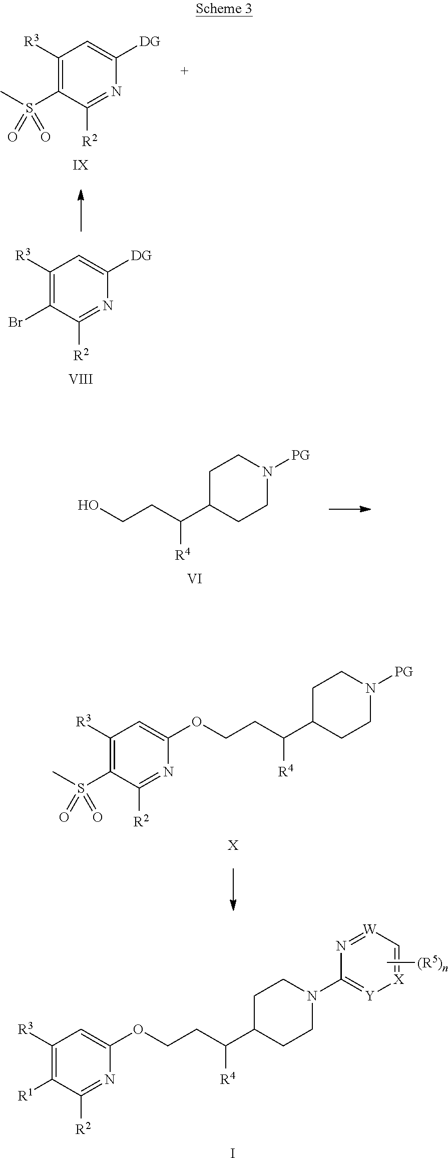

Compounds of formula (I) where R¹ is —CONHR⁶ can also be produced as outlined in Scheme 4. Reaction of compounds of formula (VI) under suitable conditions with compounds of formula (XI) yields compounds of formula (XII). An example of suitable conditions is Mitsunobu conditions using a suitable solvent such as THF, at between 0° C. and room temperature, followed by the addition of triphenylphosphine and diisopropylazodicarboxylate. Removal of the protecting group, using conditions well known to those with skill in the art, followed by reaction with an appropriate 6-membered heteroaromatic halide, under suitable conditions, yields compounds of formula (XIII). An example of suitable conditions is standard displacement conditions in DMSO in the presence of 1,8-diazabicyclo[5.4.0]undec-7-ene. Hydrolysis of the alkyl ester (XIII) under standard hydrolysis conditions, for example LiOH in MeOH and H₂O, yields the carboxylic acids (XIV). A subsequent amide bond formation, under standard conditions, well known to those with skill in the art, yields compounds of formula (I) where R¹ is —CONHR⁶ as described above.

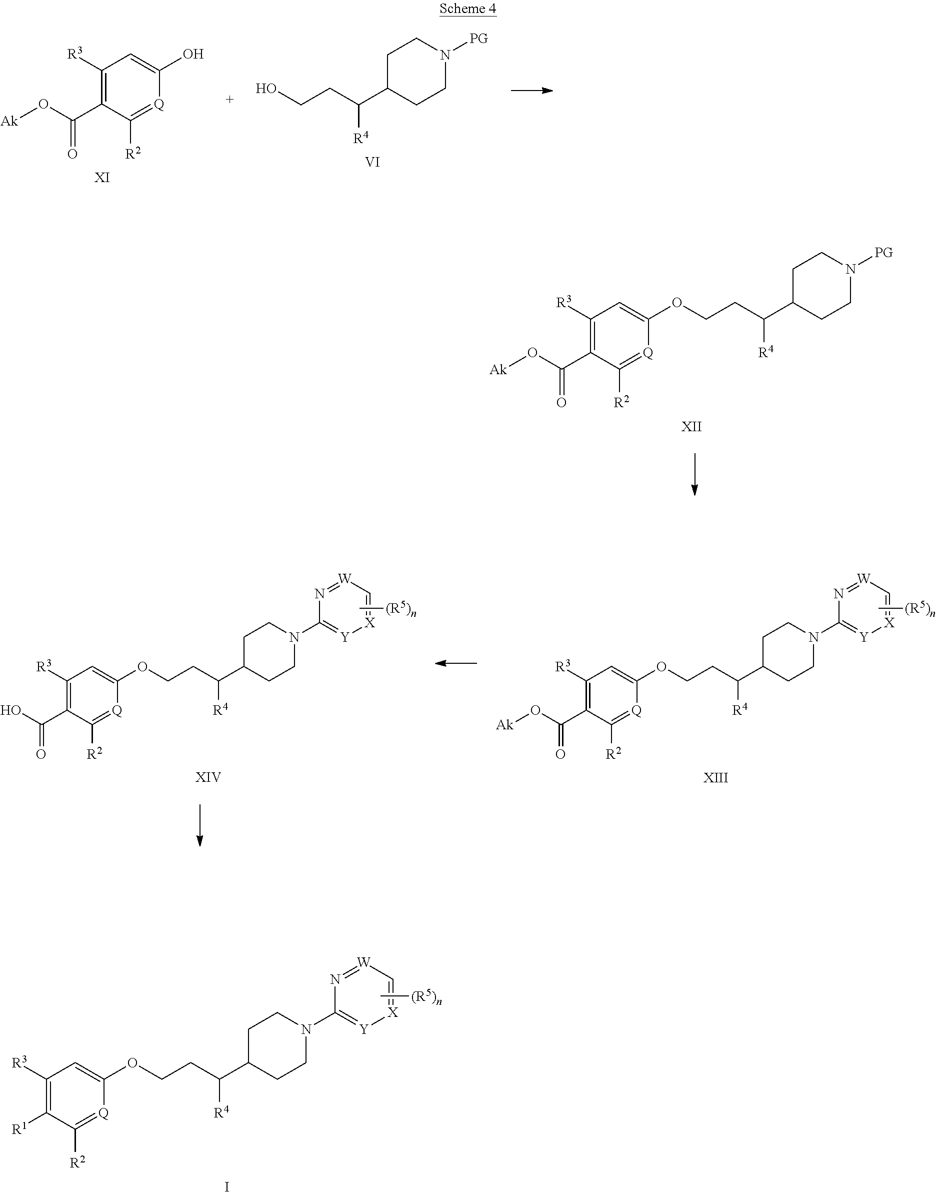

Also, compounds of formula (I), where Q is CH, R¹ is —CONHR⁶ and R² and R³ are Me can be produced as outlined in Scheme 5. Reaction of compounds of formula (XV) with 4-bromo-3,5-dimethylphenol in sulfolane with K₂CO₃ at 85° C., yields compounds of formula (XVI). Subsequent reaction with n-butyllithium in THF at −78° C., followed by quenching with CO₂, yields compounds of formula (XIV). Reaction of compounds of formula (XVII) with an appropriate 6-membered heteroaromatic halide, under suitable conditions, yields compounds of formula (XVIII). Examples of suitable conditions are standard displacement conditions in DMSO in the presence of 1,8-diazabicyclo[5.4.0]undec-7-ene, or Buchwald coupling conditions using, for example, bis(dibenzylideneacetone)palladium, NaOt-Bu, and 2,8,9-triisobutyl-2,5,8,9-tetraaza-1-phosphabicyclo[3.3.3]undecane. A subsequent amide bond formation, under standard conditions, well known to those with skill in the art, yields compounds of formula (I), where Q is CH, R¹ is —CONHR⁵ and R² and R³ are Me as described above.

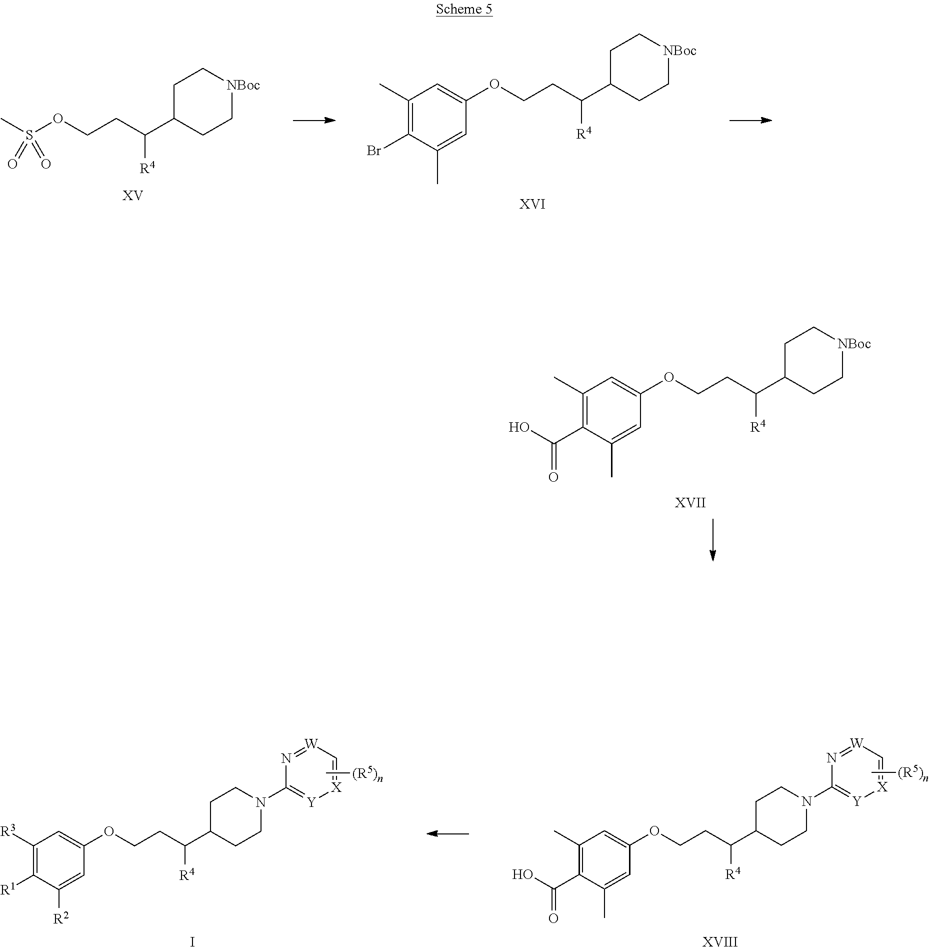

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates, such as those defined above, may be of use in the synthesis of compounds of formula (I) and are therefore also included within the scope of the invention, for example compounds of any one of formulae (VII), (X), (XIII), (XIV) and (XVIII) or a salt or protected derivative thereof.

The processes for the production of compounds of formula (I) described above also represent further aspects of the invention.

As indicated above the compounds of formula (I) are useful as GPR119 agonists, e.g. for the treatment and/or prophylaxis of obesity and diabetes. For such use the compounds of formula (I) will generally be administered in the form of a pharmaceutical composition.

The invention also provides a compound of formula (I), or a pharmaceutically acceptable salt thereof, for use as a pharmaceutical.

The invention also provides a pharmaceutical composition comprising a compound of formula (I), in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by modulating GPR119, resulting in the prophylactic or therapeutic treatment of obesity, e.g. by regulating satiety, or for the treatment of diabetes, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I) may be used in the treatment of diseases or conditions in which GPR119 plays a role.

Thus the invention also provides a method for the treatment of a disease or condition in which GPR119 plays a role comprising a step of administering to a subject in need thereof an effective amount of a compound of formula (I), or a pharmaceutically acceptable salt thereof. Diseases or conditions in which GPR119 plays a role include obesity and diabetes. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound and diabetes (including Type 1 and Type 2 diabetes, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The compounds of the invention may offer advantages over compounds acting via different mechanisms for the treatment of the above mentioned disorders in that they may offer beta-cell protection, increased cAMP and insulin secretion and also slow gastric emptying.

The compounds of the invention may also be used for treating conditions characterised by low bone mass such asosteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine and loss of height.

The invention also provides a method for the regulation of satiety comprising a step of administering to a subject in need thereof an effective amount of a compound of formula (I), or a pharmaceutically acceptable salt thereof.

The invention also provides a method for the treatment of obesity comprising a step of administering to a subject in need thereof an effective amount of a compound of formula (I), or a pharmaceutically acceptable salt thereof.

The invention also provides a method for the treatment of diabetes, including Type 1 and Type 2 diabetes, particularly type 2 diabetes, comprising a step of administering to a patient in need thereof an effective amount of a compound of formula (I), or a pharmaceutically acceptable salt thereof.

The invention also provides a method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of formula (I), or a pharmaceutically acceptable salt thereof.

The invention also provides a compound of formula (I), or a pharmaceutically acceptable salt thereof, for use in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I) may exhibit advantageous properties compared to known GPR119 agonists, for example, the compounds may exhibit improved potency or half-life, or solubility or metabolic profiles, or improved anti-obesity effects, or other advantageous properties for compounds to be used as pharmaceuticals.

The compounds of formula (I), or pharmaceutically acceptable salts thereof, may be administered alone or in combination with one or more other therapeutically active compounds.

The other therapeutically active compounds may be for the treatment of the same disease or condition as the compounds of formula (I) or a different disease or condition. The therapeutically active compounds may be administered simultaneously, sequentially or separately.

The compounds of formula (I) may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, RXR agonists, fatty acid oxidation inhibitors, α-glucosidase inhibitors, dipeptidyl peptidase IV inhibitors, GLP-1 agonists e.g. GLP-1 analogues and mimetics, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of formula (I), or a pharmaceutically acceptable salt thereof, and at least one other antiobesity agent represents a further aspect of the invention.

The present invention also provides a method for the treatment of obesity in a mammal, such as a human, which method comprises administering an effective amount of a compound of formula (I), or a pharmaceutically acceptable salt thereof, and another antiobesity agent, to a mammal in need thereof.

The invention also provides the use of a compound of formula (I), or a pharmaceutically acceptable salt thereof, and another antiobesity agent for the treatment of obesity.

The invention also provides the use of a compound of formula (I), or a pharmaceutically acceptable salt thereof, in the manufacture of a medicament for use in combination with another antiobesity agent, for the treatment of obesity.

The compound of formula (I), or a pharmaceutically acceptable salt thereof, and the other antiobesity agent(s) may be co-administered or administered sequentially or separately.

Co-administration includes administration of a formulation which includes both the compound of formula (I), or a pharmaceutically acceptable salt thereof, and the other antiobesity agent(s), or the simultaneous or separate administration of different formulations of each agent. Where the pharmacological profiles of the compound of formula (I), or a pharmaceutically acceptable salt thereof, and the other antiobesity agent(s) allow it, coadministration of the two agents may be preferred.

The invention also provides the use of a compound of formula (I), or a pharmaceutically acceptable salt thereof, and another antiobesity agent in the manufacture of a medicament for the treatment of obesity.

The invention also provides a pharmaceutical composition comprising a compound of formula (I), or a pharmaceutically acceptable salt thereof, and another antiobesity agent, and a pharmaceutically acceptable carrier. The invention also encompasses the use of such compositions in the methods described above.

GPR119 agonists are of particular use in combination with centrally acting antiobesity agents.

The other antiobesity agent for use in the combination therapies according to this aspect of the invention is preferably a CB-1 modulator, e.g. a CB-1 antagonist or inverse agonist. Examples of CB-1 modulators include SR141716 (rimonabant) and SLV-319 ((4S)-(−)-3-(4-chlorophenyl)-N-methyl-N-[(4-chlorophenyl)sulfonyl]-4-phenyl-4,5-dihydro-1H-pyrazole-1-carboxamide); as well as those compounds disclosed in EP576357, EP656354, WO 03/018060, WO 03/020217, WO 03/020314, WO 03/026647, WO 03/026648, WO 03/027076, WO 03/040105, WO 03/051850, WO 03/051851, WO 03/053431, WO 03/063781, WO 03/075660, WO 03/077847, WO 03/078413, WO 03/082190, WO 03/082191, WO 03/082833, WO 03/084930, WO 03/084943, WO 03/086288, WO 03/087037, WO 03/088968, WO 04/012671, WO 04/013120, WO 04/026301, WO 04/029204, WO 04/034968, WO 04/035566, WO 04/037823 WO 04/052864, WO 04/058145, WO 04/058255, WO 04/060870, WO 04/060888, WO 04/069837, WO 04/069837, WO 04/072076, WO 04/072077, WO 04/078261 and WO 04/108728, and the references disclosed therein.

Other diseases or conditions in which GPR119 has been suggested to play a role include those described in WO 00/50562 and U.S. Pat. No. 6,468,756, for example cardiovascular disorders, hypertension, respiratory disorders, gestational abnormalities, gastrointestinal disorders, immune disorders, musculoskeletal disorders, depression, phobias, anxiety, mood disorders and Alzheimer's disease.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh) unless specified otherwise. LCMS data were obtained as follows: Method A: Atlantis 3μC₁₈ column (3.0×20.0 mm, flow rate=0.85 mL/min) eluting with a H₂O—CH₃CN solution containing 0.1% HCO₂H over 6 min with UV detection at 220 nm. Gradient information: 0.0-0.3 min 100% H₂O; 0.3-4.25 min: Ramp up to 10% H₂O-90% CH₃CN; 4.25-4.4 min: Ramp up to 100% CH₃CN; 4.4-4.9 min: Hold at 100% CH₃CN; 4.9-6.0 min: Return to 100% H₂O. The mass spectra were obtained using an electrospray ionisation source in either the positive (ES⁺) or negative (ES⁻) ion modes; Method B: Waters Xterra MS C18, 5 μm (4.6×50 mm, flow rate 1.5 mL/min) eluting with a H₂O-MeCN gradient containing 0.1% v/v ammonia over 12 min with UV detection at 215 and 254 nm. Gradient information: 0.0-8.0 min: Ramp from 95% H₂O-5% MeCN to 5% H₂O-95% MeCN; 8.0-9.9 min: Hold at 5% H₂O-95% MeCN; 9.9-10.0 min: Return to 95% H₂O-5% MeCN; 10.0-12.0 min: Hold at 95% H₂O-5% MeCN. Mass spectra were obtained using an electrospray ionization source in either the positive (ES⁺) or negative (ES⁻) mode.

Abbreviations and acronyms: Ac: Acetyl; t-Bu: tent-Butyl; Boc: tert-butyloxycarbonyl; DBU: 1,8-Diazabicyclo[5.4.0]undec-7-ene; DCM: Dichloromethane; DEAD: Diethyl azodicarboxylate; DIAD: Diisopropyl azodicarboxylate; DIPEA: N,N-Diisopropylethylamine; DMF: Dimethylformamide; DMSO: Dimethylsulfoxide; EDCI: 1-(3-Dimethylaminopropyl)-3-ethylcarbodiimide hydrochloride; Et: Ethyl; HATU: O-(7-Azabenzotriazol-1-yl)-N,N,N′N′-tetramethyluronium hexafluorophosphate; HOAt: 1-Hydroxy-7-azabenzotriazole; HOBt: 1-Hydroxybenzotriazole; HPLC: High performance liquid chromatography; h: hour(s); IH: Isohexane; iPr: Isopropyl; Me: Methyl; min: minute/s; MP: Macroporous polystyrene; PE-AX column: silica based quaternary amine column; Ph: Phenyl; RP-HPLC: Reverse phase-high performance liquid chromatography; RT: Retention time; SCX column: strong cation exchange column (silica bound tosic acid column); THF: Tetrahydrofuran.

The syntheses of the following compounds have been described elsewhere: tert-butyl 4-((E)-2-ethoxycarbonyl-1-methylvinyl)piperidine-1-carboxylate: U.S. Pat. No. 6,518,423; tert-butyl 4-(3-hydroxypropyl)piperidine-1-carboxylate: Tetrahedron 1999, 55, 11619-11639; 2-chloro-5-isopropylpyrimidine: WO03/074495; (+)-(4S,1′R)-2,2-dimethyl-4-(1′-aminoethyl)-1,3-dioxolane: Liebigs Ann./Recueil, 1997, 1089-1100; 5-ethyl-2-fluoropyridine: US2005/277681; 4-(3-methanesulfonyloxypropyl)piperidine-1-carboxylic acid tert-butyl ester: WO98/07703; 6-hydroxy-2,4-dimethylnicotinic acid ethyl ester: Tetrahedron 1970, 26, 4641-4648; 6-hydroxy-2-methylnicotinic acid ethyl ester: Tetrahedron, 1974, 30, 623-32; 3-piperidin-4-yl-propan-1-ol: Tetrahedron 1999, 55, 11619-11639. All other compounds were available from commercial sources.

### Preparation 1: tert-Butyl 4-((E)-2-carboxy-1-methylvinyl)piperidine-1-carboxylate

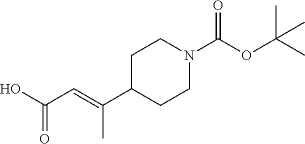

A solution of tert-butyl 4-((E)-2-ethoxycarbonyl-1-methylvinyl)piperidine-1-carboxylate (18.7 g, 62.9 mmol) in MeOH (90 mL) and H₂O (25 mL) was treated with 2M NaOH (94.5 mL, 189 mmol). The reaction was stirred for 16 h, the MeOH was removed under reduced pressure, then the remainder was partitioned between EtOAc and H₂O. The aqueous layer was separated and acidified to pH 2 with 12M HCl, before being extracted with EtOAc (2×). The organic extracts were washed with brine, dried (MgSO₄), filtered, and concentrated, then the remainder was recrystallised from EtOAc-IH to provide the title compound: m/z (ES⁻)=268.3 [M−H]⁻ (Method A).

### Preparation 2: tert-Butyl 4-((R)-2-carboxy-1-methylethyl)piperidine-1-carboxylate

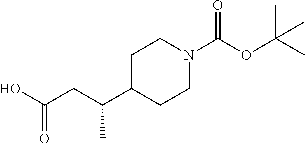

tert-Butyl 4-((E)-2-carboxy-1-methylvinyl)piperidine-1-carboxylate (Preparation 1, 130 g, 0.483 mol) was placed in a hydrogenation flask under an Ar atmosphere, then degassed MeOH (400 mL) was added. [Rh(norbornadiene)₂]BF₄ (1.80 g, 4.81 mmol) and (S)-1-[(R)-2-(di-tert-butylphosphino)ferrocenyl]ethylbis(2-methylphenyl)phosphine (2.90 g, 5.08 mmol) were placed in a separate Schlenk flask under Ar, before being treated with degassed MeOH (200 mL). This catalyst mixture was stirred for 15 min at ambient temperature, before being transferred via cannula into the hydrogenation flask. The Schlenk flask was rinsed with more degassed MeOH (100 mL). These washings were transferred to the hydrogenation flask, then more degassed MeOH (300 mL) was added. The hydrogenation flask was sealed, the Ar replaced by H₂, and the pressure set to 1.05 bar. The reaction mixture was heated to 35° C., and stirring/shaking was started. After 48 h, the reaction was stopped and a representative sample of the reaction mixture was analysed by HPLC and ¹H NMR. The conversion was 100% and the enantiomeric purity of the crude (R)-acid was 98.2%, as ascertained by the following HPLC method: Column: CHIRALPAK AD-H (previously used with CF₃CO₂H-containing solvents) 4.6×250 mm; Solvent: C₆H₁₄-iPrOH (97:3 isocratic); Temperature: 20° C.; Flow rate: 1 mL/min; UV-detection (210, 230 nm); Sample: 100 μL reaction solution dissolved with 1 mL MeOH. Retention times: (S)-acid: 19.3 min, (R)-acid: 20.6 min, starting enoic acid: 22.1 min. Isolation procedure: The MeOH was evaporated, then the crude hydrogenation product was dissolved in t-BuOMe and extracted with aqueous NaOH. The aqueous phase was added to a mixture of 1M HCl and EtOAc. The aqueous phase was extracted further with EtOAc, then the combined organic extracts were washed with brine and dried (MgSO₄). The title compound was isolated following filtration and complete removal of the solvent.

### Preparation 3: tert-Butyl 4-((R)-3-hydroxy-1-methylpropyl)piperidine-1-carboxylate

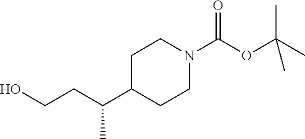

BH₃.THF (1M, 15.7 mL, 15.7 mmol) was added dropwise over 5 min to a stirred solution of tert-butyl 4-((R)-2-carboxy-1-methylethyl)piperidine-1-carboxylate (Preparation 2, 1.70 g, 6.30 mmol) in anhydrous THF at 0° C. After 1 h, the reaction was treated with Et₂O, then with 2M HCl. The organic layer was washed with brine, before being dried (Na₂SO₄). Filtration, solvent evaporation, and column chromatography (EtOAc—CH₂Cl₂, 1:3) provided the title compound: RT=3.17 min; m/z (ES⁺)=258.1 [M+H]⁺ (Method A).

### Preparation 4: 4-[(R)-3-(4-Methanesulfonylphenoxy)-1-methylpropyl]piperidine-1-carboxylic acid tert-butyl ester

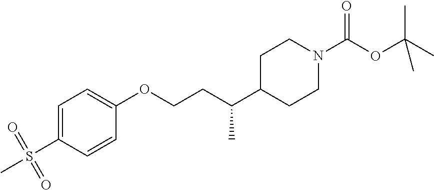

DEAD (10.8 mL, 68.4 mmol) was added to a stirred solution of tert-butyl 4-((R)-3-hydroxy-1-methylpropyl)piperidine-1-carboxylate (Preparation 3, 8.00 g, 31.1 mmol), 4-methanesulfonylphenol (5.63 g, 32.7 mmol) and PPh₃ (10.60 g, 40.4 mmol) in anhydrous THF (300 mL) at 0° C. After stirring at ambient temperature for 0.5 h, the solvent was removed in vacuo, and the remainder was dissolved in EtOAc to give a solution that was washed with 2M NaOH (2×) and brine. The organic layer was dried (MgSO₄), concentrated under reduced pressure and the remainder was triturated with IH-Et₂O. The solid produced was filtered and washed with Et₂O. The combined washings and filtrate were concentrated under reduced pressure and the residue was purified by column chromatography (EtOAc-IH, 3:7) to afford the title compound: RT=4.09 min; m/z (ES⁺)=412.00 [M+H]⁺ (Method A).

### Preparation 5: 4-[(R)-3-(4-Methanesulfonylphenoxy)-1-methylpropyl]piperidine hydrochloride

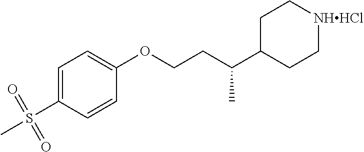

A mixture of 4-[(R)-3-(4-methanesulfonylphenoxy)-1-methylpropyl]piperidine-1-carboxylic acid tert-butyl ester (Preparation 4, 15.50 g, 37.7 mmol) and 4M HCl in dioxane (150 mL) was stirred at ambient temperature for 1 h. The solvent was removed in vacuo, azeotroping with toluene (2×), to afford the title compound: RT=2.19 min; m/z (ES⁺)=311.93 [M+H]⁺ (Method A).

### Preparation 6: 4-[3-(4-Methoxycarbonyl-3-methylphenoxy)propyl]piperidine-1-carboxylic acid tert-butyl ester

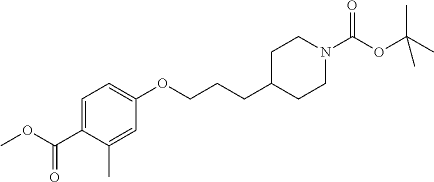

DIAD (8.00 mL, 40.9 mmol) was added to a stirred solution of 4-hydroxy-2-methylbenzoic acid methyl ester (6.00 g, 37.4 mmol), tert-butyl 4-(3-hydroxypropyl)piperidine-1-carboxylate (8.25 g, 34.0 mmol) and PPh₃ (10.71 g, 40.9 mmol) in anhydrous THF (60 mL) at ambient temperature. After stirring for 7.5 h, the solvent was removed in vacuo, and the remainder was dissolved in EtOAc and washed with 2M NaOH (2×) and brine. The organic layer was dried (MgSO₄), concentrated under reduced pressure and the remainder was triturated with IH-Et₂O. The solid produced was filtered and washed with Et₂O. The combined washings and filtrate were concentrated under reduced pressure and purified by column chromatography (EtOAc-IH, 1:9) to afford the title compound: RT=4.48 min; m/z (ES⁺)=392.3 [M+H]⁺ (Method A).

### Preparation 7: 4-[3-(4-Carboxy-3-methylphenoxy)propyl]piperidine-1-carboxylic acid tert-butyl ester

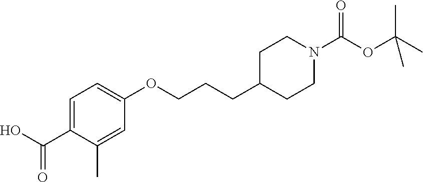

To a solution of 4-[3-(4-methoxycarbonyl-3-methylphenoxy)propyl]piperidine-1-carboxylic acid tert-butyl ester (Preparation 6, 6.00 g, 15.3 mmol) in MeOH (200 mL) and H₂O (20 mL) was added LiOH.H₂O (6.43 g, 153.3 mmol) and the resulting mixture was stirred at 40° C. for 16 h. The MeOH was evaporated off under reduced pressure, then the remainder was dissolved in H₂O (200 mL), washed with EtOAc and acidified to pH 4 with 2M HCl, before being extracted with EtOAc (2×). The combined organic extracts were washed with brine, dried (MgSO₄), filtered, and concentrated in vacuo to yield the title compound RT=4.06 min; m/z (ES⁺)=378.22 [M+H]⁺ (Method A).

### Preparation 8: 4-{3-[4-(2-Hydroxyethylcarbamoyl)-3-methylphenoxy]propyl}piperidine-1-carboxylic acid tert-butyl ester

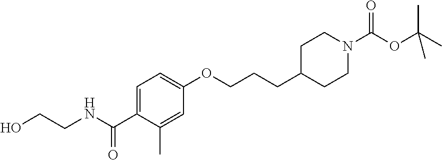

HOBt.H₂O (77.0 mg, 660 μmol) was added to a stirred solution of 4-[3-(4-carboxy-3-methylphenoxy)propyl]piperidine-1-carboxylic acid tert-butyl ester (Preparation 7, 200 mg, 530 μmol) and EDCI (126 mg, 660 μmol) in THF (10 mL). After 1 h, 2-aminoethanol (64 μL, 1.06 mmol) was added and the resulting mixture was stirred at ambient temperature for 16 h. The reaction mixture was diluted with DCM, washed with 1M NaOH, dried through a hydrophobic fit and concentrated in vacuo. Purification by column chromatography (EtOAc-IH, 4:1) afforded the title compound: RT=3.54 min; m/z (ES⁺)=421.14 [M+H]⁺ (Method A).

### Preparation 9: N-(2-Hydroxyethyl)-2-methyl-4-(3-piperidin-4-ylpropoxy)benzamide hydrochloride

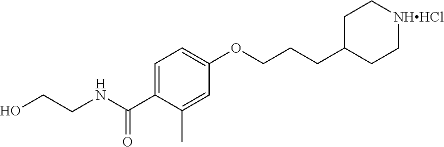

This compound was prepared from 4-{3-[4-(2-hydroxyethylcarbamoyl)-3-methyl-phenoxy]propyl}piperidine-1-carboxylic acid tert-butyl ester (Preparation 8, 710 mg, 1.69 mmol) using a procedure similar to that outlined in Preparation 5: RT=2.02 min; m/z (ES⁺)=321.10 [M+H]⁺ (Method A).

### Preparation 10: 2-Methyl-4-(3-piperidin-4-yl-propoxy)benzoic acid hydrochloride

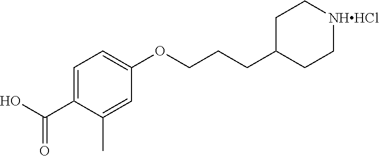

This compound was prepared from 4-[3-(4-carboxy-3-methylphenoxy)propyl]-piperidine-1-carboxylic acid tert-butyl ester (Preparation 7, 126 mg, 340 μmol) using a procedure similar to that outlined in Preparation 5: RT=2.37 min; ink (ES⁺)=278.17 [M+H]⁺ (Method A).

### Preparation 11: 4-{3-[1-(5-Chloropyrimidin-2-yl)piperidin-4-yl]propoxy}-2-methylbenzoic acid methyl ester

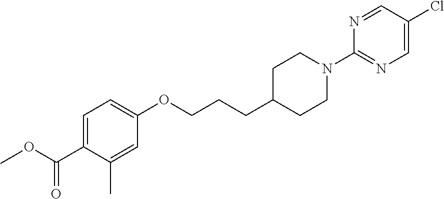

4M HCl in dioxane (7.7 mL) was added to a stirred solution of 4-[3-(4-methoxycarbonyl-3-methylphenoxy)propyl]piperidine-1-carboxylic acid tert-butyl ester (Preparation 6, 4.00 g, 10.2 mmol) in dioxane (10 mL) at ambient temperature. After 3 h, the mixture was diluted with Et₂O and the solid product formed was collected by filtration and washed with Et₂O to afford the hydrochloride salt of 2-methyl-4-(3-piperidin-4-ylpropoxy)-benzoic acid methyl ester: RT=2.65 min; ink (ES⁺)=292.4 [M+H]⁺ (Method A). To a stirred solution of this compound (1.27 g, 3.89 mmol) in DMSO (12 mL) was added 2,5-dichloropyrimidine (580 mg, 3.89 mmol) and DBU (1.25 mL, 8.54 mmol) and the resulting solution was stirred at 100° C. for 16 h. The reaction mixture was diluted with H₂O and extracted with EtOAc (2×), then the combined organic extracts were washed with brine, before being dried (MgSO₄). Filtration, removal of solvent under reduced pressure and purification by column chromatography (EtOAc-IH, 1:19) afforded the title compound: RT=4.80 min; m/z (ES⁺)=404.15 [M+H]⁺ (Method A).

### Preparation 12: 4-{3-[1-(5-Chloropyrimidin-2-yl)piperidin-4-yl]propoxy}-2-methylbenzoic acid

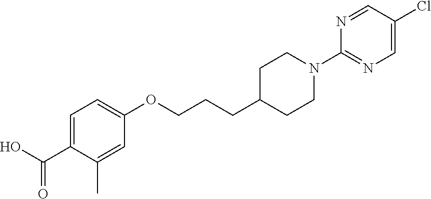

A mixture of LiOH.H₂O (308 mg, 7.33 mmol) and 4-{3-[1-(5-chloropyrimidin-2-yl)-piperidin-4-yl]propoxy}-2-methylbenzoic acid methyl ester (Preparation 11, 1.41 g, 3.49 mmol) in THF (48 mL) and H₂O (4.8 mL) was heated at 65° C. for 96 h. The THF was removed under reduced pressure, then the remainder was partitioned between 2M NaOH and EtOAc. The aqueous phase was acidified to pH 1 with 12M HCl, before being extracted with EtOAc (2×). The combined organic extracts were washed with brine, dried (MgSO₄), filtered and concentrated in vacuo to afford the title compound: RT=4.27 min; m/z (ES⁺)=390.15 [M+H]⁺ (Method A).

### Preparation 13: 4-[3-(5′-Ethyl-3,4,5,6-tetrahydro-2H-[1,2′]bipyridinyl-4-yl)propoxy]-2-methylbenzoic acid methyl ester

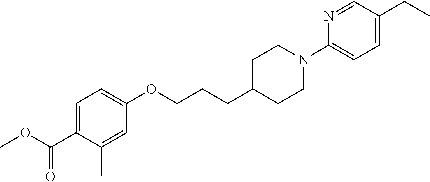

The title compound was synthesized from 4-[3-(4-methoxycarbonyl-3-methyl-phenoxy)propyl]piperidine-1-carboxylic acid tert-butyl ester (Preparation 6) and 5-ethyl-2-fluoropyridine employing the procedures outlined in Preparation 11: RT=3.18 min; m/z (ES⁺)=397.20 [M+H]⁺ (Method A).

### Preparation 14: 4-[3-(5′-Ethyl-3,4,5,6-tetrahydro-2H-[1,2′]bipyridinyl-4-yl)propoxy]-2-methylbenzoic acid

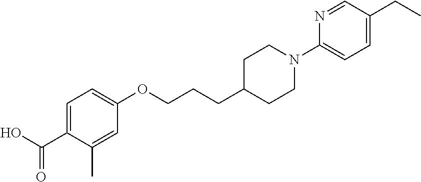

2M NaOH (18.9 mL, 37.9 mmol) was added to a solution of 4-[3-(5′-ethyl-3,4,5,6-tetrahydro-2H-[1,2′]bipyridinyl-4-yl)propoxy]-2-methylbenzoic acid methyl ester (Preparation 13, 1.50 g, 3.79 mmol) in MeOH (30 mL) and the resulting solution was stirred at ambient temperature for 16 h. The reaction mixture was acidified to pH 6 with 2M HCl and concentrated in vacuo to afford the title compound: RT=2.90 min; m/z (ES⁺)=38118 [M+H]⁺ (Method A).

### Preparation 15: 4-{3-[1-(5-Ethylpyrimidin-2-yl)piperidin-4-yl]propoxy}-2-methylbenzoic acid methyl ester

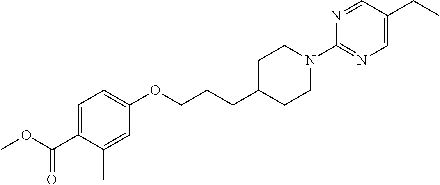

The title compound was synthesized from 4-[3-(4-methoxycarbonyl-3-methyl-phenoxy)propyl]piperidine-1-carboxylic acid tert-butyl ester (Preparation 6) and 5-ethyl-2-chloropyrimidine employing the procedures outlined in Preparation 11: RT=4.51 min; m/z (ES⁺)=398.83 [M+H]⁺ (Method A).

### Preparation 16: 4-{3-[1-(5-Ethylpyrimidin-2-yl)piperidin-4-yl]propoxy}-2-methylbenzoic acid

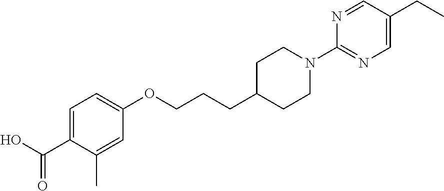

A mixture of LiOH.H₂O (63.3 mg, 1.51 mmol) and 4-{3-[1-(5-ethylpyrimidin-2-yl)-piperidin-4-yl]propoxy}-2-methylbenzoic acid methyl ester (Preparation 15, 60.0 mg, 1.51 mmol) in MeOH (2 mL) and H₂O (200 pt) was heated at 50° C. for 5 h. The MeOH was removed under reduced pressure, then the remainder was partitioned between H₂O and EtOAc. The aqueous phase was acidified to pH 4 with 2M HCl, before being extracted with EtOAc (2×). The combined organic extracts were washed with brine, dried (MgSO₄), filtered and concentrated in vacuo to afford the title compound: RT=3.80 min; m/z (ES⁺)=384.34 [M+H]⁺ (Method A).

### Preparation 17: 4-[3-(4-Bromo-3,5-dimethylphenoxy)propyl]piperidine-1-carboxylic acid tert-butyl ester

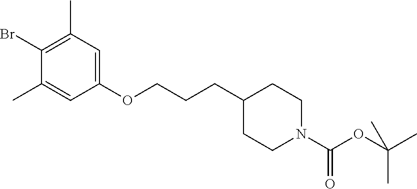

4-Bromo-3,5-dimethylphenol (13.75 g, 68.4 mmol) and K₂CO₃ (18.90 g, 136.8 mmol) were added to a solution of 4-(3-methanesulfonyloxypropyl)piperidine-1-carboxylic acid tert-butyl ester (21.98 g, 68.4 mmol) in sulfolane (260 mL) and the resulting solution was heated at 85° C. for 4 h. The reaction mixture was diluted with Et₂O (500 mL) and H₂O (500 mL) and the organic layer was washed with H₂O (4×), 2M NaOH (2×) and brine, before being dried (MgSO₄). Filtration, solvent removal and purification by column chromotagraphy (DCM) furnished the title compound: RT=4.94 min; m/z (ES⁺)=426.20 [M+H]⁺ (Method A).

### Preparation 18: 4-[3-(4-Carboxy-3,5-dimethylphenoxy)propyl]piperidine-1-carboxylic acid tert-butyl ester

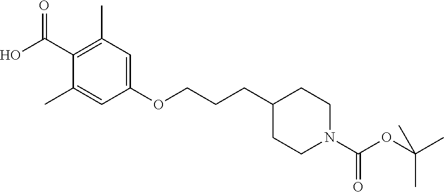

To a solution of 1.6 M n-butyllithium in hexane (20.64 mL, 51.6 mmol) in anhydrous THF (23 mL) at −78° C. under argon, was added a solution of 4-[3-(4-bromo-3,5-dimethyl-phenoxy)propyl]piperidine-1-carboxylic acid tert-butyl ester (Preparation 17, 11.00 g, 25.8 mmol) in anhydrous THF (34 mL). The reaction mixture was stirred at −78° C. for 50 min, then CO₂ gas was bubbled through the reaction mixture as it warmed to ambient temperature (˜0.5 h). The reaction mixture was quenched with H₂O and diluted with EtOAc. The organic layer was extracted with 2M NaOH (2×) and the combined basic extracts were combined with the aqueous layer. The aqueous was acidified to pH 1 with 2M HCl and extracted with EtOAc (3×), then the combined organic extracts were washed with brine and dried (MgSO₄). Filtration, solvent removal and purification by column chromatography ((EtOAc-IH, 3:7)) furnished the title compound: RT=3.93 min; m/z (ES⁺)=392.23 [M+H]⁺ (Method A).

### Preparation 19: 2,6-Dimethyl-4-(3-piperidin-4-ylpropoxy)benzoic acid hydrochloride

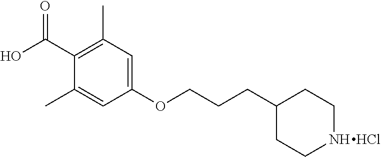

4M HCl in dioxane (21.95 mL) was added to a stirred solution of 4-[3-(4-carboxy-3,5-dimethylphenoxy)propyl]piperidine-1-carboxylic acid tert-butyl ester (Preparation 18, 4.91 g, 12.5 mmol) in dioxane (20 mL) at ambient temperature. After 2.5 h, the solid product that had formed was collected by filtration and washed with Et₂O to afford the title compound: RT=2.50 min; m/z (ES⁺)=291.40 [M+H]⁺ (Method A).

### Preparation 20: 4-{3-[1-(5-Chloropyrimidin-2-yl)piperidin-4-yl]propoxy}-2,6-dimethyl-benzoic acid

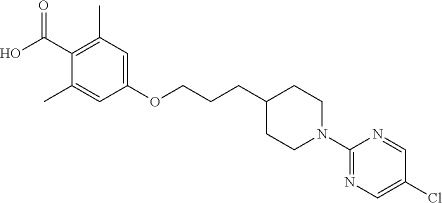

To 2,6-dimethyl-4-(3-piperidin-4-ylpropoxy)benzoic acid hydrochloride (Preparation 19, 600 mg, 1.83 mmol) in DMSO (850 mL) was added 2,5-dichloropyrimidine (327 mg, 2.20 mmol), DBU (960 μL, 6.41 mmol) and H₂O (6 drops). The resulting suspension was heated in a sealed tube in the microwave at 130° C. for 3 h. The reaction mixture was diluted with H₂O, acidified to pH 5 with 2M HCl and extracted with EtOAc (3×), then the combined organic extracts were washed with brine, before being dried (MgSO₄). Filtration, removal of solvent under reduced pressure and purification by column chromatography (EtOAc-IH, 2:3 to 3:2) afforded the title compound: RT=4.20 min; m/z (ES⁺)=404.16 [M+H]⁺ (Method A).

The benzoic acids listed in Table 1 were synthesised from 2,6-dimethyl-4-(3-piperidin-4-ylpropoxy)benzoic acid hydrochloride (Preparation 19) and either 5-chloro-2-fluoropyridine or the appropriate 2-chloropyrimidine employing a procedure similar to that outlined in Preparation 20.

TABLE 1

| Prep. | Structure | Name | Spectra: LCMS Method A |
|---|---|---|---|
| 21 | 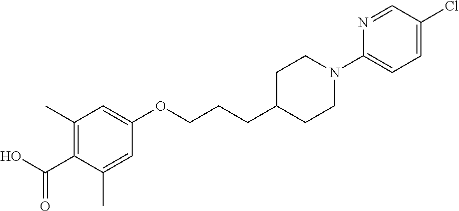 | 4-[3-(5′-chloro-3,4,5,6- tetrahydro-2H- [1,2′]bipyridinyl-4-yl)- propoxy]-2,6-dimethyl- benzoic acid | RT = 3.87 min; m/z (ES⁺) = 403.11 [M + H]⁺ |
| 22 | 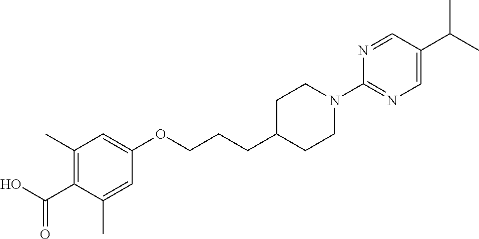 | 4-{3-[1-(5-Isopropyl- pyrimidin-2-yl)- piperidin-4-yl]- propoxy}-2,6-dimethyl- benzoic acid | RT = 3.79 min; m/z (ES⁺) = 412.14 [M + H]⁺ |
| 23 | 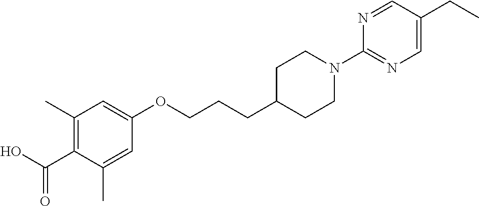 | 4-{3-[1-(5-Ethyl- pyrimidin-2-yl)- piperidin-4-yl]- propoxy}-2,6-dimethyl- benzoic acid | RT = 3.95 min; m/z (ES⁺) = 398.22 [M + H]⁺ |
| 24 | 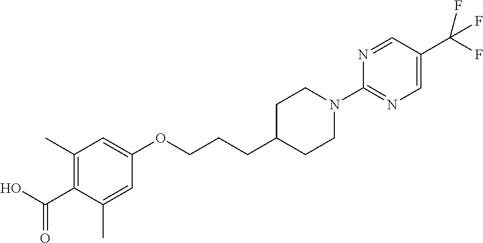 | 2,6-Dimethyl-4-{3-[1- (5-trifluoromethyl- pyrimidin-2-yl)- piperidin-4-yl]- propoxy}benzoic acid | RT = 4.53 min; m/z (ES⁺) = 438.15 [M + H]⁺ |

### Preparation 25: 4-[3-(5′-Isopropyl-3,4,5,6-tetrahydro-2H-[1,2′]bipyridinyl-4-yl)propoxy]-2,6-dimethylbenzoic acid

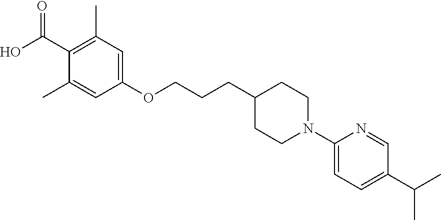

To a solution of 2,6-dimethyl-4-(3-piperidin-4-ylpropoxy)benzoic acid hydrochloride (Preparation 19, 280 mg, 853 μmol) in dioxane (4 mL) was added 2-chloro-5-isopropyl-pyridine (1.59 g, 1.02 mmol), bis(dibenzylideneacetone)palladium (78.1 mg, 85.0 μmol), NaOt—Bu (287 mg, 2.99 mmol) and 2,8,9-triisobutyl-2,5,8,9-tetraaza-1-phosphabicyclo[3.3.3]undecane (29.2 mg, 85.0 μmol). Argon was bubbled through the reaction mixture for 0.5 h, then the reaction mixture was heated in a sealed tube in the microwave at 115° C. for 1.5 h. The reaction was diluted with methanol, filtered through celite and concentrated in vacuo. The remainder was diluted with H₂O, acidified to pH 5 with 2M HCl and extracted with EtOAc (2×). The combined organics were washed with brine, dried (MgSO₄), concentrated in vacuo and purified by column chromatography (EtOAc-IH, 3:2) to afford the title compound: RT=2.98 min; m/z (ES⁺)=411.21 [M+H]⁺ (Method A).

### Preparation 26: 4-[3-(5′-Isopropyl-3,4,5,6-tetrahydro-2H-[1,2′]bipyridinyl-4-yl)propoxy]-2-methylbenzoic acid

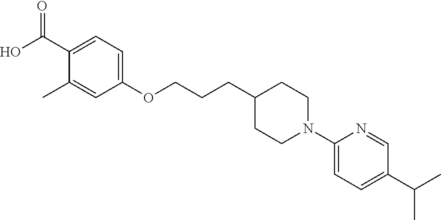

The title compound was synthesized from 2-methyl-4-(3-piperidin-4-ylpropoxy)-benzoic acid hydrochloride (Preparation 10, 100 mg, 320 μmol) employing a procedure similar to that outlined in Preparation 25: RT=3.05 min; m/z (ES⁺)=397.22 [M+H]⁺ (Method A).

The benzoic acids listed in Table 2 were synthesized from 2-methyl-4-(3-piperidin-4-ylpropoxy)benzoic acid hydrochloride (Preparation 10), and the appropriate 5-fluoropyridine, 5-chloropyridine or 5-ethylpyrimidine employing a procedure similar to that outlined in Preparation 20.

TABLE 2

| Prep. | Structure | Name | Spectra: LCMS Method A |
|---|---|---|---|
| 27 | 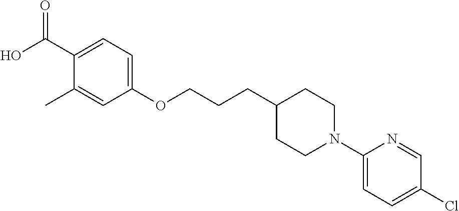 | 4-[3-(5′-Chloro-3,4,5,6- tetrahydro-2H- [1,2′]bipyridinyl-4-yl)- propoxy]-2-methyl- benzoic acid | RT = 3.90 min; m/z (ES⁺) = 389.10 [M +H]⁺ |
| 28 | 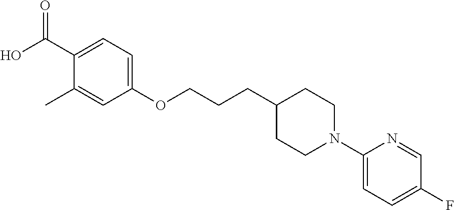 | 4-[3-(5′-Fluoro-3,4,5,6- tetrahydro-2H- [1,2′]bipyridinyl-4-yl)- propoxy]-2-methyl- benzoic acid | RT = 3.55 min; m/z (ES⁺) = 373.15 [M +H]⁺ |
| 29 | 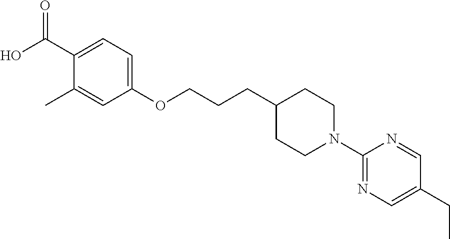 | 4-{3-[1-(5-Ethyl- pyrimidin-2-yl)- piperidin-4-yl]- propoxy}-2-methyl- benzoic acid | RT = 3.78 min; m/z (ES⁺) = 384.20 [M +H]⁺ |

### Preparation 30: 4-((R)-3-Methanesulfonyloxy-1-methylpropyl)piperidine-1-carboxylic acid tert-butyl ester

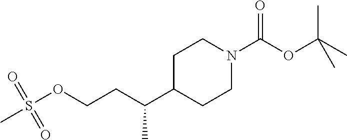

NEt₃ (13.0 mL, 93.4 mmol) was added to a stirred solution of tert-butyl 4-((R)-3-hydroxy-1-methylpropyl)piperidine-1-carboxylate (Preparation 3, 20.0 g, 77.9 mmol) and methanesulfonyl chloride (9.80 g, 85.6 mmol) in DCM (200 mL). The reaction mixture was warmed to ambient temperature and stirred for 3 h. Further methanesulfonyl chloride (5.00 g, 43.7 mmol) and NEt₃ (6.90 mL, 49.5 mmol) were added and stirring at ambient temperature was continued for 10 h. The reaction mixture was partitioned between DCM and H₂O, then the organic phase was washed with 1M HCl, 1M NaOH and brine, dried (MgSO₄), filtered and concentrated in vacuo. Purification by column chromotography (EtOAc-DCM, 1:19) afforded the title compound: RT=3.65 min; m/z (ES⁺)=336.19 [M+H]⁺ (Method A).

### Preparation 31: 4-[(R)-3-(4-Methoxycarbonyl-3-methylphenoxy)-1-methylpropyl]piperidine-1-carboxylic acid tert-butyl ester

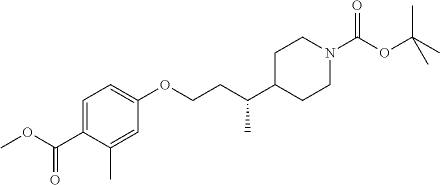

The title compound was synthesized from 4-hydroxy-2-methylbenzoic acid methyl ester (8.10 g, 48.7 mmol) and 4-((R)-3-methanesulfonyloxy-1-methylpropyl)piperidine-1-carboxylic acid tert-butyl ester (Preparation 30, 16.3 g, 48.7 mmol) employing a procedure similar to that outlined in Preparation 17: RT=4.55 min; ink (ES⁺)=406.30 [M+H]⁺ (Method A).

### Preparation 32: 2-Methyl-4-((R)-3-piperidin-4-yl-butoxy)benzoic acid hydrochloride

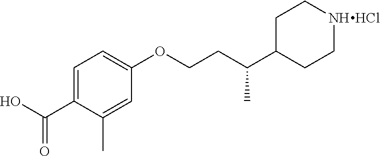

To a solution of 4-[(R)-3-(4-methoxycarbonyl-3-methylphenoxy)-1-methylpropyl]-piperidine-1-carboxylic acid tert-butyl ester (Preparation 31, 10.0 g, 24.7 mmol) in MeOH (100 mL) and H₂O (10 mL) was added LiOH.H₂O (10.4 g, 246.9 mmol) and the resulting mixture was stirred at 50° C. for 16 h. The MeOH was evaporated off under reduced pressure, then the remainder was dissolved in H₂O and acidified to pH 1 with 1M HCl, before being extracted with DCM (2×). The combined organic extracts were dried (MgSO₄), filtered, and concentrated in vacuo to yield 4-[(R)-3-(4-carboxy-3-methylphenoxy)-1-methylpropyl]piperidine-1-carboxylic acid tert-butyl ester: RT=4.10 min; m/z (ES⁺)=392.30 [M+H]⁺ (Method A). This compound was stirred with 4M HCl in dioxane for 2 h. Removal of the solvent in vacuo afforded the title compound: RT=2.43 min; m/z (ES⁺)=292.18 [M+H]⁺ (Method A).

### Preparation 33: 4-{(R)-3-[1-(5-Chloropyrimidin-2-yl)piperidin-4-yl]butoxy}-2-methylbenzoic acid

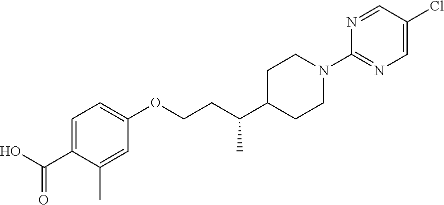

The title compound was synthesized from 2-methyl-4-((R)-3-piperidin-4-ylbutoxy)-benzoic acid hydrochloride (Preparation 32, 500 mg, 1.53 mmol) and 2,5-dichloropyrimidine (270 mg, 1.84 mmol) employing a procedure similar to that outlined in Preparation 20: RT=4.39 min; m/z (ES⁺)=404.14 [M+H]⁺ (Method A).

### Preparation 34: 4-{(R)-3-[1-(5-Ethylpyrimidin-2-yl)piperidin-4-yl]butoxy}-2-methylbenzoic acid

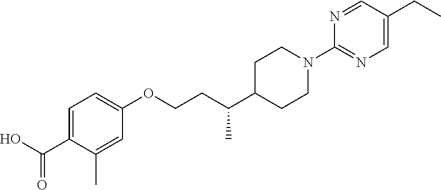

The title compound was synthesized from 2-methyl-4-((R)-3-piperidin-4-ylbutoxy)-benzoic acid hydrochloride (Preparation 32, 500 mg, 1.53 mmol) and 2-chloro-5-ethyl-pyrimidine (260 mg, 1.84 mmol) employing a procedure similar to that outlined in Preparation 20: RT=3.92 min; m/z (ES⁺)=398.21 [M+H]⁺ (Method A).

### Preparation 35: 5-tert-Butyl-2-chloropyridine

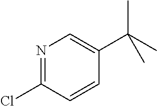

3,3-Dimethylbutyraldehyde (6.30 mL, 50.0 mmol) was added dropwise over 20 min to a stirring solution of morpholine (4.00 mL, 46.0 mmol) in anhydrous cyclohexane (40 mL) under argon. The resulting solution was heated at 80° C. for 16 h, before cooling to ambient temperature and adding EtOAc (40 mL), hydroquinone (20.0 mg) and acetic acid (0.3 mL). The reaction mixture was heated to 78° C. and 2-chloroacrylnitrile (5.54 mL, 69.0 mmol) was added dropwise over 20 min. Heating at 78° C. was continued for 1 h, then HCl gas was bubbled through the reaction mixture for 15 min, before cooling to ambient temperature. Saturated aqueous NaHCO₃ was added to adjust the reaction mixture to pH 8, then the organic phase was separated and the aqueous extracted with EtOAc (2×). The combined organics were washed with brine, dried (MgSO₄), filtered and concentrated in vacuo. Purification by column chromatography (EtOAc-IH, 1:9) afforded the title compound: RT=142 min; m/z (ES⁺)=170.06 [M+H]⁺ (Method A).

### Preparation 36: 4-[3-(5′-tert-Butyl-3,4,5,6-tetrahydro-2H-[1,2′]bipyridinyl-4-yl)propoxy]-2-methylbenzoic acid

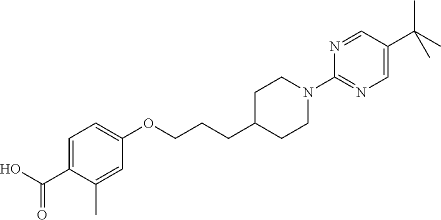

The title compound was synthesized from 2-methyl-4-(3-piperidin-4-ylpropoxy)-benzoic acid hydrochloride (Preparation 10, 400 mg, 1.28 mmol) and 5-tert-butyl-2-chloropyridine (Preparation 35, 302 mg, 1.79 mmol) employing a procedure similar to that outlined in Preparation 25: RT=3.07 min; m/z (ES⁺)=411.16 [M+H]⁺ (Method A).

### Preparation 37: 4-{3-[1-(5-Isopropylpyrimidin-2-yl)piperidin-4-yl]propoxy}-2-methylbenzoic acid methyl ester

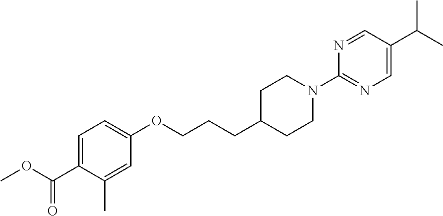

The title compound was synthesized from 4-[3-(4-methoxycarbonyl-3-methyl-phenoxy)propyl]piperidine-1-carboxylic acid tert-butyl ester (Preparation 6) and 2-chloro-5-isopropylpyrimidine employing the procedures outlined in Preparation 11: RT=4.44 min; m/z (ES⁺)=412.22 [M+H]⁺ (Method A).

### Preparation 38: 4-{3-[1-(5-Isopropylpyrimidin-2-yl)piperidin-4-yl]propoxy}-2-methylbenzoic acid

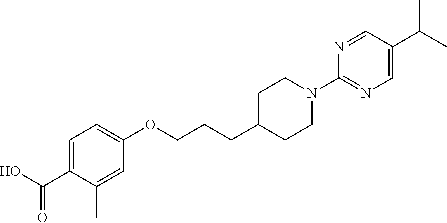

To a solution of 4-{3-[1-(5-isopropylpyrimidin-2-yl)piperidin-4-yl]propoxy}-2-methyl-benzoic acid methyl ester (Preparation 37, 726 mg, 1.76 mmol) in MeOH (17 mL) and H₂O (3 mL) was added NaOH (704 mg, 17.6 mmol) and the resulting mixture was stirred at 50° C. for 16 h. THF (5 mL) was added to the reaction mixture and heating was continued for 72 h. The reaction mixture was cooled to ambient temperature, acidified to pH 1 with 2M HCl and extracted with DCM and EtOAc. The combined organic extracts were concentrated in vacuo to yield the title compound: RT=3.90 min; m/z (ES⁺)=398.19 [M+H]⁺ (Method A).

### Preparation 39: 4-[(R)-3-(4-Bromo-3,5-dimethylphenoxy)-1-methylpropyl]piperidine-1-carboxylic acid tert-butyl ester

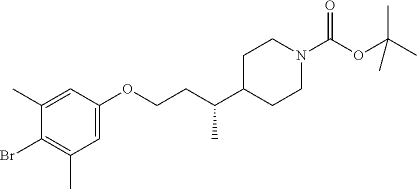

Reaction of 4-bromo-3,5-dimethylphenol with 4-((R)-3-methanesulfonyloxy-1-methylpropyl)piperidine-1-carboxylic acid tert-butyl ester (Preparation 30) employing a procedure similar to that utilized in Preparation 17 furnished the title compound: RT=5.40 min; m/z (ES⁺)=440.18 [M+H]⁺ (Method A).

### Preparation 40: 4-[(R)-3-(4-Carboxy-3,5-dimethylphenoxy)-1-methylpropyl]piperidine-1-carboxylic acid tert-butyl ester

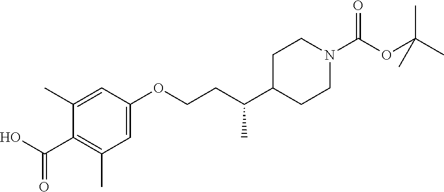

Using a procedure similar to that employed in Preparation 18, 4-[(R)-3-(4-bromo-3,5-dimethylphenoxy)-1-methylpropyl]piperidine-1-carboxylic acid tert-butyl ester (Preparation 39) was transformed into the corresponding aryllithium, which was then reacted with CO₂ to furnish the title compound: RT=4.28 min; m/z (ES⁻)=404.45 [M−H]⁻ (Method A).

### Preparation 41: 2,6-Dimethyl-4-((R)-3-piperidin-4-ylbutoxy)benzoic acid hydrochloride

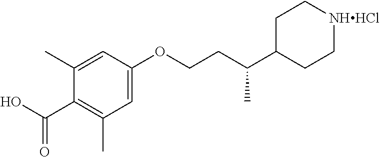

A stirred solution of 4-[(R)-3-(4-carboxy-3,5-dimethylphenoxy)-1-methylpropyl]-piperidine-1-carboxylic acid tert-butyl ester (Preparation 40, 200 mg, 0.49 mmol) in CH₂Cl₂ (3 mL) at 0° C. was treated with a solution of TFA (2 mL) in CH₂Cl₂ (3 mL). After 2 h, the reaction mixture was concentrated under reduced pressure, before being stirred with CH₂Cl₂ (3 mL) and 1M HCl (2 mL). After 1 h, the mixture was concentrated in vacuo to afford the title compound: RT=2.13 min; m/z (ES⁺)=306.15 [M+H]⁺ (Method A).

### Preparation 42: 4-{(R)-3-[1-(5-Chloropyrimidin-2-yl)piperidin-4-yl]butoxy}-2,6-dimethyl-benzoic acid

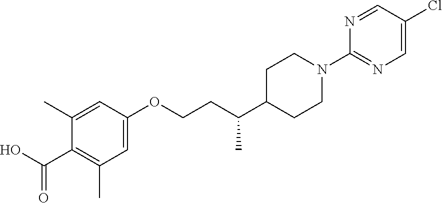

Reaction of 2,6-dimethyl-4-((R)-3-piperidin-4-ylbutoxy)benzoic acid hydrochloride (Preparation 41) with 2,5-dichloropyrimidine, utilizing a procedure similar to that outlined in Preparation 11, yielded the title compound: RT=4.24 min; m/z (ES⁺)=418.22 [M+H]⁺ (Method A).

### Preparation 43: 6-[(R)-3-(1-tert-Butoxycarbonylpiperidin-4-yl)butoxy]-2-methylnicotinic acid ethyl ester

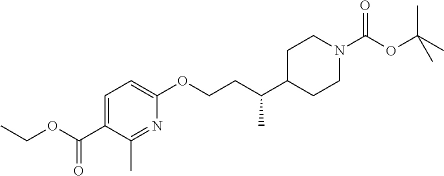

DIAD (5.20 mL, 20.6 mmol) was added to a stirred solution of tert-butyl 4-((R)-3-hydroxy-1-methylpropyl)piperidine-1-carboxylate (Preparation 3, 4.56 g, 17.7 mmol), 6-hydroxy-2-methylnicotinic acid ethyl ester (4.44 g, 26.6 mmol) and PPh₃ (6.97 g, 26.6 mmol) in anhydrous THF (50 mL) at ambient temperature. After stirring for 30 min, the solvent was removed in vacuo, and the remainder was dissolved in EtOAc and washed with 2M NaOH (2×) and brine. The organic layer was dried (MgSO₄), concentrated under reduced pressure to approximately one fifth of the original volume and IH was added. The solid produced was removed by filtration and the filtrate was concentrated under reduced pressure and purified by column chromatography (EtOAc-IH, 3:17) to afford the title compound: RT=4.75 min; m/z (ES⁺)=421.28 [M+H]⁺ (Method A).

### Preparation 44: 6-[(R)=1-(1-tert-Butoxycarbonylpiperidin-4-yl)butoxy]2-methylnicotinic acid

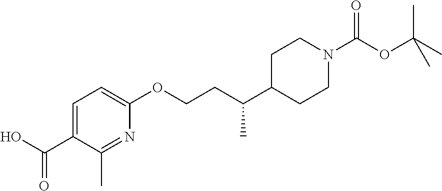

LiOH.H₂O (3.00 g, 71.4 mmol) was added to a stirred solution of 6-[(R)-3-(1-tert-butoxycarbonylpiperidin-4-yl)butoxy]-2-methylnicotinic acid ethyl ester (Preparation 43, 3.00 g, 7.14 mmol) in MeOH (40 mL) and H₂O (4 mL) and the resulting suspension was stirred at 60° C. for 16 h. The solvent was removed in vacuo, then the remainder was dissolved in H₂O, acidified to pH 1 with 2M HCl and extracted with DCM (3×). The combined organic extracts were dried (MgSO₄), filtered and concentrated in vacuo to afford the title compound: RT=4.02 min; ink (ES⁺)=393.28 [M+H]⁺ (Method A).

### Preparation 45: 6-{(R)-3-[1-(5-Chloropyrimidin-2-yl)piperidin-4-yl]butoxy}-2-methylnicotinic acid

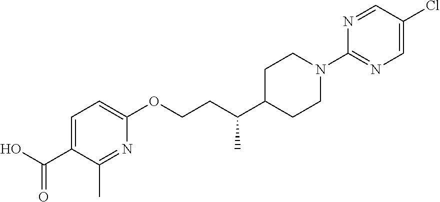

4M HCl in dioxane (100 mL) was added to 6-[(R)-3-(1-tert-butoxycarbonylpiperidin-4-yl)butoxy]-2-methylnicotinic acid (Preparation 44, 2.70 g, 6.89 mmol) and the resulting solution was stirred at ambient temperature for 2 h. The resulting solid was collected by filtration and washed with Et₂O to afford the hydrochloride salt of 2-methyl-6-((R)-3-piperidin-4-ylbutoxy)nicotinic acid: RT=2.43 min; ink (ES⁺)=292.18 [M+H]⁺. To a stirred solution of this compound (500 mg, 1.37 mmol) in DMSO (1 mL) was added 2,5-dichloropyrimidine (410 mg, 2.74 mmol) and DBU (814 μL, 5.48 mmol) and the resulting solution was heated at 100° C. in a sealed tube in the microwave for 1.5 h. The reaction mixture was diluted with 10% aqueous citric acid and EtOAc, then the organic phase was separated and washed with H₂O (3×) and brine (3×), before being dried (MgSO₄). Filtration, removal of solvent under reduced pressure and purification by recrystallisation (EtOAc) afforded the title compound: RT=4.63 min; ink (ES⁺)=405.12 [M+H]⁺ (Method A).

### Preparation 46: 6-{3-[1-(5-Isopropylpyrimidin-2-yl)piperidin-4-yl]propoxy}-2-methylnicotinic acid ethyl ester

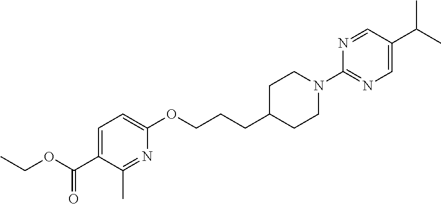

4M HCl in dioxane (10 mL) was added to 6-[(R)-3-(1-tert-butoxycarbonylpiperidin-4-yl)butoxy]-2-methylnicotinic acid ethyl ester (Preparation 43, 2.00 g, 4.80 mmol) and the resulting solution was stirred at ambient temperature for 4 h. The solvent was removed in vacuo to afford the hydrochloride salt of 2-methyl-6-(3-piperidin-4-ylpropoxy)nicotinic acid ethyl ester. To a stirred solution of this compound in DMSO (20 mL) was added 2-chloro-5-isopropyl-pyrimidine (357 mg, 2.29 mmol) and DBU (1.52 g, 11.0 mmol) and the resulting solution was heated at 100° C. for 16 h. The solvent was removed in vacuo at 80° C. and the remainder was dissolved in DCM and washed with saturated NaHCO₃ solution and brine, before being dried (MgSO₄), filtered and concentrated in vacuo. Purification by column chromatography (EtOAc-IH, 1:4) afforded the title compound: RT=4.64 min; ink (ES⁺)=427.23 [M+H]⁺ (Method A).

### Preparation 47: 6-{3-[1-(5-Isopropylpyrimidin-2-yl)piperidin-4-yl]propoxy}-2-methylnicotinic acid

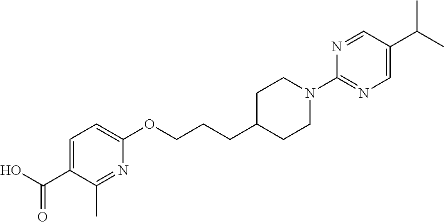

To a solution of 6-{3-[1-(5-isopropylpyrimidin-2-yl)piperidin-4-yl]propoxy}-2-methylnicotinic acid ethyl ester (Preparation 46, 137 mg, 322 μmol) in MeOH (4.2 mL) and H₂O (0.8 mL) was added NaOH (133 mg, 3.22 mmol) and the resulting mixture was stirred at 50° C. for 18 h. The reaction mixture was cooled to ambient temperature, acidified to pH 1 with 2M HCl and extracted with DCM and EtOAc. The combined organic extracts were concentrated in vacuo to yield the title compound: RT=3.54 min; m/z (ES⁺)=399.21 [M+H]⁺ (Method A).

### Preparation 48: 6-[3-(1-tert-Butoxycarbonylpiperidin-4-yl)propoxy]-2-methylnicotinic acid ethyl ester

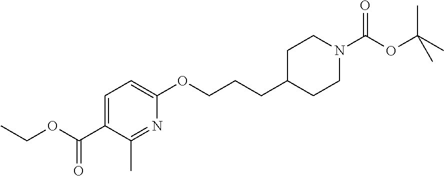

The title compound was synthesized from tert-butyl 4-(3-hydroxypropyl)piperidine-1-carboxylate (5.00 g, 2.05 mmol) and 6-hydroxy-2-methylnicotinic acid ethyl ester (4.47 g, 2.47 mmol) employing a procedure similar to that outlined in Preparation 43: RT=4.60 min; m/z (ES⁺)=407.24 [M+H]⁺ (Method A).

### Preparation 49: 6-[3-(1-tert-Butoxycarbonylpiperidin-4-yl)propoxy]-2-methylnicotinic acid

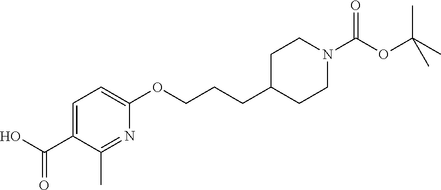

The title compound was synthesized from 6-[3-(1-tert-butoxycarbonylpiperidin-4-yl)-propoxy]-2-methylnicotinic acid ethyl ester (Preparation 48, 7.07 g, 1.74 mmol) employing a procedure similar to that employed in Preparation 44: RT=3.96 min; m/z (ES⁺)=379.16 [M+H]⁺ (Method A).

### Preparation 50: N-(2-Hydroxy-1-hydroxymethylethyl)-2-methyl-6-(3-piperidin-4-ylpropoxy)-nicotinamide hydrochloride

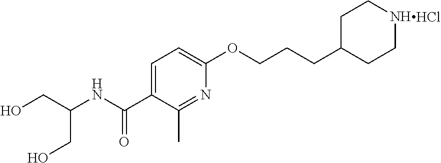

HOBt.H₂O (771 mg, 5.71 mmol) was added to a stirred solution of 6-[3-(1-tert-butoxycarbonylpiperidin-4-yl)propoxy]-2-methylnicotinic acid (Preparation 49, 1.80 g, 4.76 mmol), EDCI (1.10 g, 5.71 mmol) and DIPEA (2.50 mL, 14.3 mmol) in THF (50 mL). After 15 min, 2-aminopropane-1,3-diol (650 mg, 7.13 mmol) was added and the resulting mixture was stirred at ambient temperature for 16 h. The THF was removed in vacuo and the residue dissolved in DCM (200 mL) and washed with 1M NaOH, H₂O and brine, before being dried (MgSO₄). Filtration, solvent evaporation, and purification by column chromatography (EtOAc-MeOH, 25:1) afforded 4-{3-[5-(2-hydroxy-1-hydroxymethylethylcarbamoyl)-6-methylpyridin-2-yloxy]propyl}piperidine-1-carboxylic acid tert-butyl ester: RT=3.28 min; m/z (ES⁺)=452.29 [M+H]⁺. To this compound was added 4M HCl in dioxane (2.8 mL) and the resulting solution was stirred at ambient temperature for 2 h. Removal of the solvent in vacuo afforded the title compound: RT=1.88 min; m/z (ES⁺)=352.15 [M+H]⁺ (Method A).

### Preparation 51: N—((S)-2,3-Dihydroxypropyl)-2-methyl-6-(3-piperidin-4-ylpropoxy)-nicotinamide

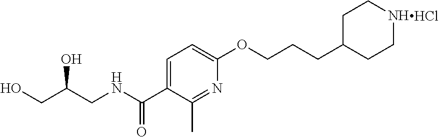

The title compound was synthesized from 6-[3-(1-tert-butoxycarbonylpiperidin-4-yl)-propoxy]-2-methylnicotinic acid (Preparation 49, 290 mg, 766 μmol) and (S)-3-amino-propane-1,2-diol (104.7 mg, 1.15 mmol) employing procedures similar to those outlined in Preparation 50: RT=1.89 min; m/z (ES)=350.34 [M−H]⁻ (Method A).

### Preparation 52: 6-{3-[1-(5-Ethylpyrimidin-2-yl)piperidin-4-yl]propoxy}-2-methylnicotinic acid

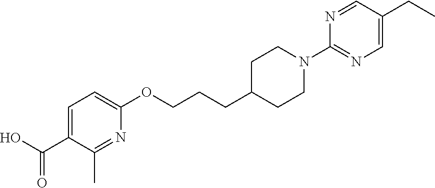

4M HCl in dioxane was added to 6-[3-O-tert-butoxycarbonylpiperidin-4-yl)propoxy]-2-methylnicotinic acid (Preparation 49, 250 mg, 6.61 mmol) and the resulting solution stirred at ambient temperature for 2 h. Removal of the solvent in vacuo afforded 2-methyl-6-(3-piperidin-4-ylpropoxy)nicotinic acid hydrochloride. To a solution of this compound in DMSO (5 mL) was added 2-chloro-5-ethylpyrimidine (222 mg, 1.56 mmol) and DBU (750 μL, 4.67 mmol) and the resulting solution was stirred at 70° C. for 72 h and at 100° C. for 3 h. The reaction mixture was diluted with MeOH (10 mL) and purified using a PE-AX column (MeOH-DCM, 1:1) to afford the title compound: RT=3.66 min; m/z (ES⁺)=385.28 [M+H]⁺ (Method A).

### Preparation 53: 6-Fluoro-3-methanesulfonyl-2-methylpyridine

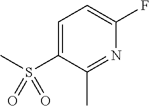

To a solution of 3-bromo-6-fluoro-2-methylpyridine (3.25 g, 17.1 mmol) and TMEDA (3.35 mL, 22.2 mmol) in toluene (200 mL) at −75° C. under argon was added 1.6 M n-butyllithium in hexane (12.8 mL, 20.5 mmol) over 10 min, and the mixture stirred for 50 min before dimethyldisulfide (1.84 mL, 20.5 mmol) was added. The reaction was stirred for 1 h at 75° C. then warmed to 2° C. and quenched with saturated NH₄Cl solution (40 mL). The organic phase was collected, washed with brine, dried (MgSO₄) and the solvent was removed under vacuum to give a residue which was purified by column chromatography (IH-EtOAc, 39:1) to furnish 6-fluoro-2-methyl-3-methylsulfanylpyridine. To a solution of this compound (330 mg, 2.10 mmol) in DCM (7 mL) at 0° C. was added 77% 3-chloroperbenzoic acid (970 mg, 4.30 mmol) over 15 min. A further aliquot of DCM (5 mL) was added and the mixture stirred for 1 h. The reaction mixture was diluted with DCM (25 mL), washed with Na₂CO₃ (15 mL) and the organic phase was collected through a hydrophobic frit. The solvent was removed under vacuum to furnish the title compound: RT=2.13 min; m/z (ES⁺)=189.89 [M+H]⁺ (Method A).

### Preparation 54: 3-Methanesulfonyl-2-methyl-6-(3-piperidin-4-ylpropoxy)pyridine hydrochloride

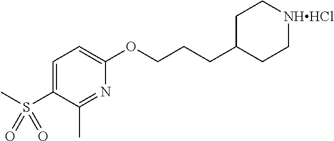

NaH (197 mg, 4.93 mmol) was added portionwise to a solution of tert-butyl 4-(3-hydroxypropyl)piperidine-1-carboxylate (1.00 g, 4.11 mmol) in THF (10 mL) and the resulting reaction mixture stirred at ambient temperature for 1 h. 6-Fluoro-3-methanesulfonyl-2-methylpyridine (Preparation 53, 778 mg, 4.11 mmol) was added to the reaction mixture and the resulting solution stirred at 70° C. for 2.5 h before quenching with H₂O. The reaction mixture was extracted with EtOAc (3×100 mL) and the combined organic extracts washed with brine, dried (MgSO₄), filtered and concentrated in vacuo. Purification by column chromatography (IH-EtOAc, 3:7) afforded 4-[3-(5-methanesulfonyl-6-methylpyridin-2-yloxy)propyl]piperidine-1-carboxylic acid tert-butyl ester: RT=4.17 min; m/z (ES⁺)=413.18 [M+H]⁺. To this compound (740 mg, 1.79 mmol) was added 4M HCl in dioxane (10 mL) and the resulting solution stirred at ambient temperature for 2 h. The solvent was removed in vacuo to afford the title compound: RT=2.21 min; m/z (ES⁺)=313.23 [M+H]⁺ (Method A).

### Preparation 55: 3-[1-(5-Chloropyrimidin-2-yl)piperidin-4-yl]propan-1-ol

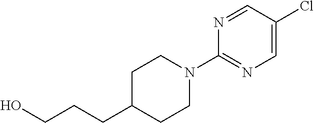

A stirred solution of 3-piperidin-4-ylpropan-1-ol hydrochloride (15.0 g, 84 mmol) in DMSO (120 mL) was cooled to 0° C., before being treated dropwise with DBU (30.0 mL, 201 mmol) over 5 min 2,5-Dichloropyrimidine (17.4 g, 117 mmol) was added portionwise, then the reaction was heated to 110° C. for 4 h. After cooling to 20° C., the reaction was poured into H₂O (200 mL) and extracted with EtOAc (3×500 mL). The combined organic extracts were washed with 1M HCl (2×200 mL), before being dried (MgSO₄) and concentrated. The residue was purified by column chromatography (EtOAc-IH, 4:6) to provide the title compound: ¹H NMR (CDCl₃) δ1.10-1.23 (m, 2H), 1.30-1.38 (m, 2H), 1.48-1.57 (m, 1H), 1.58-1.66 (m, 2H), 1.78 (d, 2H), 2.86 (m, 2H), 3.66 (t, 2H), 4.67 (d, 2H), 8.20 (s, 2H).

### Preparation 56: 6-{3-[1-(5-Chloropyrimidin-2-yl)piperidin-4-yl]propoxy}-2-methylnicotinic acid ethyl ester

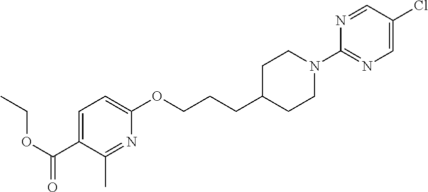

DIAD (2.88 mL, 14.7 mmol) was added dropwise over 10 min to a stirred solution of 3-[1-(5-chloropyrimidin-2-yl)piperidin-4-yl]propan-1-ol (Preparation 55, 2.50 g, 9.8 mmol), 6-hydroxy-2-methylnicotinic acid ethyl ester (1.95 g, 10.8 mmol), and PPh₃ (3.85 g, 14.7 mmol) in anhydrous THF (40 mL). After 16 h, the THF was removed under reduced pressure, then the residue was partitioned between EtOAc (200 mL) and 1M NaOH (100 mL). The organic layer was washed with brine, before being dried (MgSO₄), filtered and concentrated. The remainder was triturated with EtOAc-IH, then the filtrate was collected and purified by flash chromatography (EtOAc-IH, 1:9) to furnish the title compound: m/z (ES⁺)=419.16 [M+H]⁺ (Method A).

### Preparation 57: 6-{3-[1-(5-Chloropyrimidin-2-yl)piperidin-4-yl]propoxy}-2-methylnicotinic acid

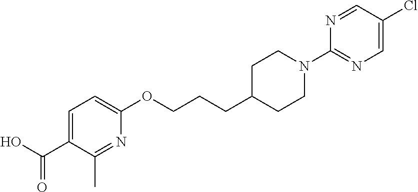

6-{3-[1-(5-Chloropyrimidin-2-yl)piperidin-4-yl]propoxy}-2-methylnicotinic acid ethyl ester (Preparation 56, 2.65 g, 6.33 mmol) was saponified, using a procedure similar to that outlined in Preparation 44, to furnish the title compound: m/z (ES⁺)=391.13 [M+H]⁺ (Method A).

### Preparation 58: 6-{3-[1-(5-Chloropyrimidin-2-yl)piperidin-4-yl]propoxy}-2,4-dimethyl-nicotinic acid ethyl ester

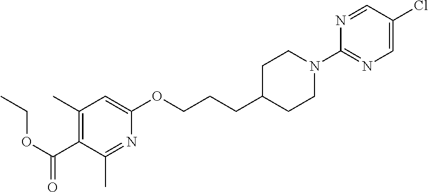

3-[1-(5-Chloropyrimidin-2-yl)piperidin-4-yl]propan-1-ol (Preparation 55, 1.55 g, 6.05 mmol) was condensed with 6-hydroxy-2,4-dimethylnicotinic acid ethyl ester (1.30 g, 6.66 mmol), employing a protocol similar to that outlined in Preparation 56, to furnish the title compound: m/z (ES⁺)=433.17 [M+H]⁺ (Method A).

### Preparation 59: 6-{3-[1-(5-Chloropyrimidin-2-yl)piperidin-4-yl]propoxy}-2,4-dimethyl-nicotinic acid

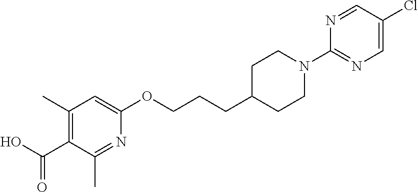

6-{3-[1-(5-Chloropyrimidin-2-yl)piperidin-4-yl]propoxy}-2,4-dimethylnicotinic acid ethyl ester (Preparation 58, 0.50 g, 1.15 mmol) was saponified, using a procedure similar to that outlined in Preparation 44, to furnish the title compound: ink (ES⁺)=405.15 [M+H]⁺ (Method A).

### Examples

The Examples in Table 3 were synthesised using the following general procedure: To a solution of 4-[(R)-3-(4-methanesulfonylphenoxy)-1-methylpropyl]piperidine hydrochloride (Preparation 5, 50.0 mg, 160 μmol) in DMSO (1 mL) was added DBU (36.0 μL, 240 μmol) and the appropriate aryl halide (240 μmol). The reaction mixture was heated overnight at 100° C. then loaded on to an SCX cartridge (2 g). The SCX cartridge was washed with MeOH, then the product was eluted with 1% NH₃ in MeOH. Evaporation of the solvent afforded the crude product that was purified using one or more of the following purification methods:

Method 1: Products were triturated with Et₂O then filtered.

Method 2: Products were purified by column chromotography (DCM-MeOH, 98:2).

Method 3: Products were dissolved in MeOH and shaken with MP-Isocyanate at ambient temperature for 1 h. Purified products were obtained upon filtration and evaporation of the solvent.

TABLE 3

| Ex | Structure | Name | Spectra |
|---|---|---|---|
| 1 | 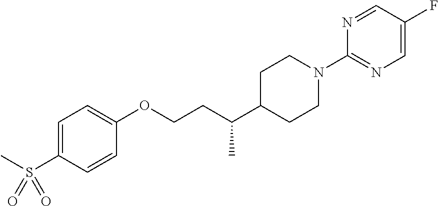 | 5-Fluoro-2-{4-[(R)-3-(4- methanesulfonyl- phenoxy)-1-methyl- propyl]piperidin-1-yl}- pyrimidine | RT = 4.23 min; m/z (ES⁺) = 408.16 [M + H]⁺ (Method B) |
| 2 | 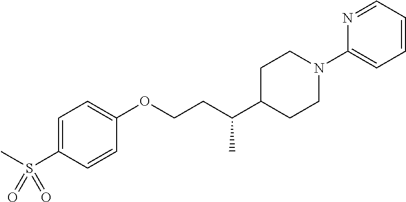 | 4-[(R)-3-(4- Methanesulfonyl- phenoxy)-1-methyl- propyl]-3,4,5,6- tetrahydro-2H- [1,2′]bipyridinyl | RT = 1.91 min; m/z (ES⁺) = 389.17 [M + H]⁺ (Method B) |
| 3 | 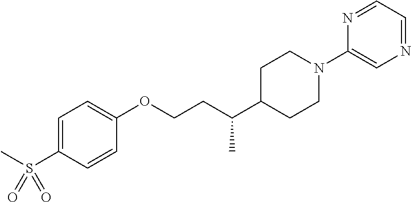 | 2-{4-[(R)-3-(4- Methanesulfonyl- phenoxy)-1-methyl- propyl]piperidin-1-yl}- pyrazine | RT = 3.38 min; m/z (ES⁺) = 390.15 [M + H]⁺ (Method B) |
| 4 | 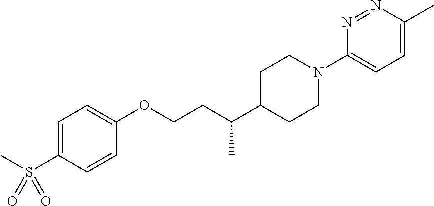 | 3-{4-[(R)-3-(4- Methanesulfonyl- phenoxy)-1-methyl- propyl]piperidin-1-yl}- 6-methylpyridazine | RT = 1.91 min; m/z (ES⁺) = 404.15 [M + H]⁺ (Method B) |
| 5 | 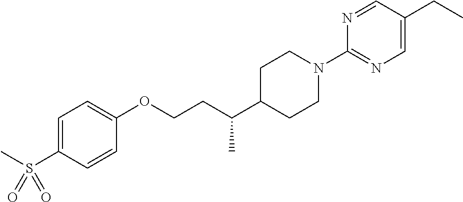 | 5-Ethyl-2-{4-[(R)-3-(4- methanesulfonyl- phenoxy)-1-methyl- propyl]piperidin-1-yl}- pyrimidine | RT = 3.90 min; m/z (ES⁺) = 418.14 [M + H]⁺ (Method B) |
| 6 | 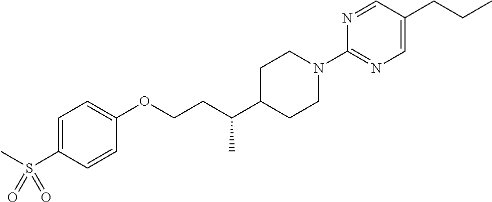 | 2-{4-[(R)-3-(4- Methanesulfonyl- phenoxy)-1-methyl- propyl]piperidin-1-yl}- 5-propylpyrimidine | RT = 4.25 min; m/z (ES⁺) = 432.18 [M + H]⁺ (Method B) |
| 7 | 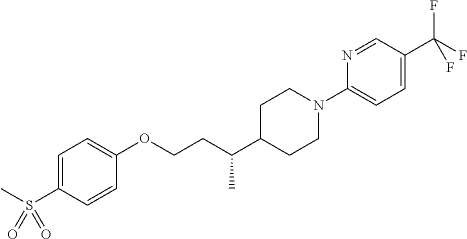 | 4-[(R)-3-(4- Methanesulfonyl- phenoxy)-1-methyl- propyl]-5′- trifluoromethyl-3,4,5,6- tetrahydro-2H- [1,2′]bipyridinyl | RT = 4.49 min; m/z (ES⁺) = 457.12 [M + H]⁺ (Method B) |
| 8 | 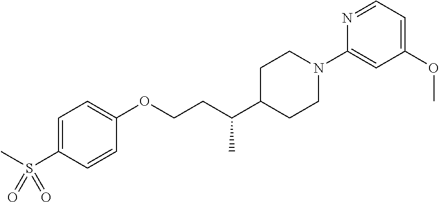 | 4-[(R)-3-(4- Methanesulfonyl- phenoxy)-1-methyl- propyl]-4′-methoxy- 3,4,5,6-tetrahydro-2H- [1,2′]bipyridinyl | RT = 2.05 min; m/z (ES⁺) = 419.13 [M + H]⁺ (Method B) |
| 9 | 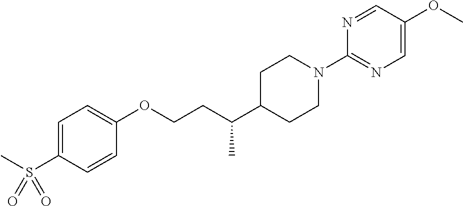 | 2-{4-[(R)-3-(4- Methanesulfonyl- phenoxy)-1-methyl- propyl]piperidin-1-yl}- 5-methoxypyrimidine | RT = 3.80 min; m/z (ES⁺) = 420.13 [M + H]⁺ (Method B) |
| 10 | 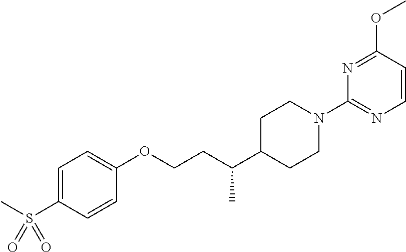 | 2-{4-[(R)-3-(4- Methanesulfonyl- phenoxy)-1-methyl- propyl]piperidin-1-yl}- 4-methoxypyrimidine | RT = 2.31 min; m/z (ES⁺) = 420.03 [M + H]⁺ (Method B) |
| 11 | 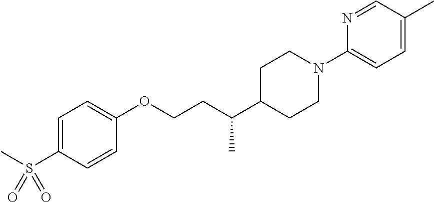 | 4-[(R)-3-(4- Methanesulfonyl- phenoxy)-1-methyl- propyl]-5′-methyl- 3,4,5,6-tetrahydro-2H- [1,2′]bipyridinyl | RT = 2.01 min; m/z (ES⁺) = 403.05 [M + H]⁺ (Method B) |
| 12 | 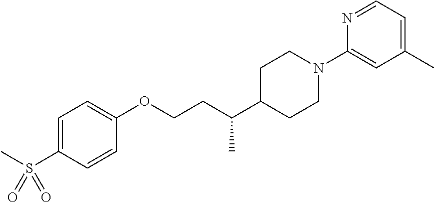 | 4-[(R)-3-(4- Methanesulfonyl- phenoxy)-1-methyl- propyl]-4′-methyl- 3,4,5,6-tetrahydro-2H- [1,2′]bipyridinyl | RT = 1.99 min; m/z (ES⁺) = 403.05 [M + H]⁺ (Method B) |
| 13 | 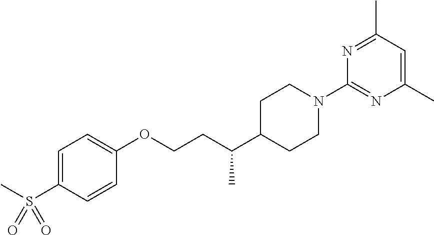 | 2-{4-[(R)-3-(4- Methanesulfonyl- phenoxy)-1-methyl- propyl]piperidin-1-yl}- 4,6-dimethylpyrimidine | RT = 2.88 min; m/z (ES⁺) = 418.01 [M + H]⁺ (Method B) |
| 14 | 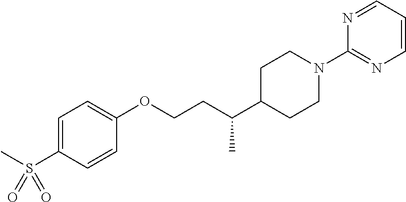 | 2-{4-[(R)-3-(4- Methanesulfonyl- phenoxy)-1-methyl- propyl]piperidin-1-yl}- pyrimidine | RT = 3.44 min; m/z (ES⁺) = 389.94 [M + H]⁺ (Method B) |
| 15 | 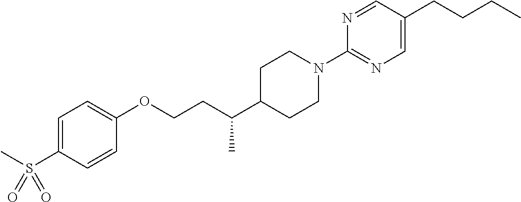 | 5-Butyl-2-{4-[(R)-3-(4- methanesulfonyl- phenoxy)-1-methyl- propyl]piperidin-1-yl}- pyrimidine | RT = 4.67 min; m/z (ES⁺) = 446.13 [M + H]⁺ (Method B) |
| 16 | 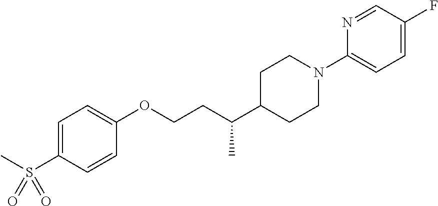 | 5′-Fluoro-4-[(R)-3-(4- methanesulfonyl- phenoxy)-1-methyl- propyl]-3,4,5,6- tetrahydro-2H- [1,2′]bipyridinyl | RT = 3.77 min; m/z (ES⁺) = 407.09 [M + H]⁺ (Method A) |
| 17 | 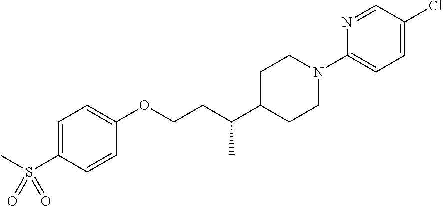 | 5′-Chloro-4-[(R)-3-(4- methanesulfonyl- phenoxy)-1-methyl- propyl]-3,4,5,6- tetrahydro-2H- [1,2′]bipyridinyl | RT = 4.12 min; m/z (ES⁺) = 423.05 [M + H]⁺ (Method A) |

### Example 18

### 4-{3-[1-(5-Chloropyrimidin-2-yl)piperidin-4-yl]propoxy}-N-(2-hydroxyethyl)-2-methylbenzamide

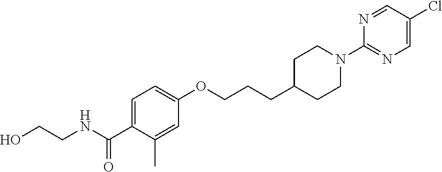

HOBt.H₂O (50.0 mg, 370 μmol) was added to a stirred solution of 4-{3-[1-(5-chloropyrimidin-2-yl)piperidin-4-yl]propoxy}-2-methylbenzoic acid (Preparation 12, 110 mg, 283 μmol) and EDCI (70.5 mg, 370 μmol) in THF (7 mL). After 20 min, 2-aminoethanol (68 μL, 1.13 mmol) was added and the resulting mixture was heated at 40° C. for 16 h. The THF was removed in vacuo and the residue partitioned between EtOAc and 2M NaOH. The organic phase was separated and washed with 2M NaOH, 1M HCl and brine, before being dried (MgSO₄). Filtration, solvent evaporation, and purification by column chromatography (EtOAc) afforded the title compound: RT=3.79 min; m/z (ES⁺)=433.24 [M+H]⁺ (Method A).

The amides listed in Table 4 were synthesised by condensing the appropriate acid with the appropriate amine, employing a procedure similar to that outlined in Example 18.

TABLE 4

| Ex | Structure | Name | Spectra: LCMS Method A |
|---|---|---|---|
| 19 | 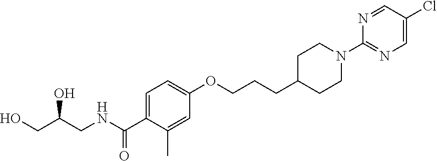 | 4-{3-[1-(5-(Chloropyrimidin- 2-yl)piperidin-4-yl]- propoxy}-N-((S)-2,3- dihydroxypropyl)-2-methyl- benzamide | RT = 3.66 min; m/z (ES⁺) = 463.22 [M + H]⁺ |
| 20 | 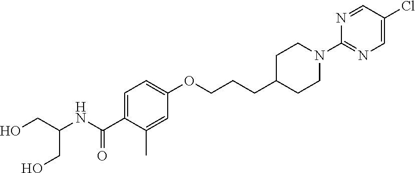 | 4-{3-[1-(5-Chloropyrimidin- 2-yl)piperidin-4-yl]- propoxy}-N-(2-hydroxy-1- hydroxymethylethyl)-2- methylbenzamide | RT = 3.66 min; m/z (ES⁺) = 463.31 [M + H]⁺ |
| 21 | 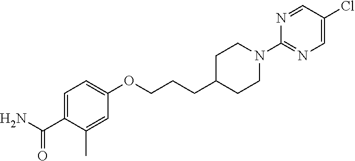 | 4-{3-[1-(5-Chloropyrimidin- 2-yl)piperidin-4-yl]- propoxy}-2-methyl- benzamide | RT = 3.92 min; m/z (ES⁺) = 389.19 [M + H]⁺ |
| 22 | 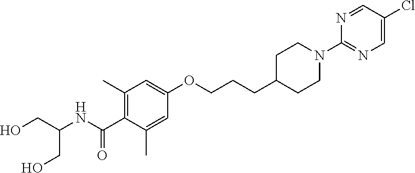 | 4-{3-[1-(5-Chloropyrimidin- 2-yl)piperidin-4-yl]- propoxy}-N-(2-hydroxy-1- hydroxymethylethyl)-2,6- dimethylbenzamide | RT = 3.60 min; m/z (ES⁺) = 477.16 [M + H]⁺ |
| 23 | 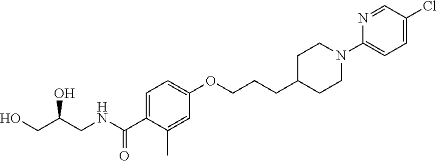 | 4-[3-(5′-Chloro-3,4,5,6- tetrahydro-2H- [1,2′]bipyridinyl-4-yl)- propoxy]-N-((S)-2,3- dihydroxypropyl)-2-methyl- benzamide | δ_{H} (CDCl₃) 1.21-1.36 (m, 2H), 1.41-1.51 (m, 2H), 1.52-1.65 (m, 1H), 1.78-1.92 (m, 4H), 2.50 (s, 3H), 2.79- 2.90 (m, 2H), 2.90-3.03 (m, 2H), 3.59-3.73 (m, 4H), 3.85-3.94 (m, 1H), 4.01 (t, 2H), 4.22-4.31 (m, 2H), 6.15- 6.24 (m, 1H), 6.62 (d, 1H), 6.71-6.80 (m, 2H), 7.36-7.45 (m, 2H), 8.11-8.15 (m, 1H); RT = 3.17 min; m/z (ES⁺) = 462.17 [M + H]⁺ |
| 24 | 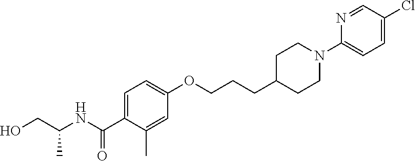 | 4-[3-(5′-Chloro-3,4,5,6- tetrahydro-2H- [1,2′]bipyridinyl-4-yl)- propoxy]-N-((R)-2-hydroxy- 1-methylethyl)-2-methyl- benzamide | RT = 3.42 min; m/z (ES⁺) = 446.20 [M + H]⁺ |
| 25 | 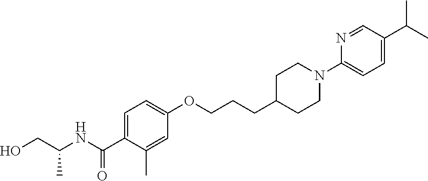 | N-((R)-2-Hydroxy-1-methyl- ethyl)-4-[3-(5′-isopropyl- 3,4,5,6-tetrahydro-2H- [1,2′]bipyridinyl-4-yl)- propoxy]-2-methyl- benzamide | RT = 2.75 min; m/z (ES⁺) = 454.27 [M + H]⁺ |
| 26 | 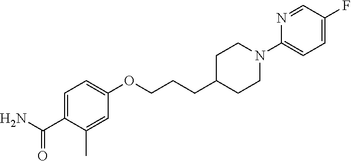 | 4-[3-(5′-Fluoro-3,4,5,6- tetrahydro-2H- [1,2′]bipyridinyl-4-yl)- propoxy]-2-methyl- benzamide | RT = 3.09 min; m/z (ES⁺) = 372.18 [M + H]⁺ |
| 27 | 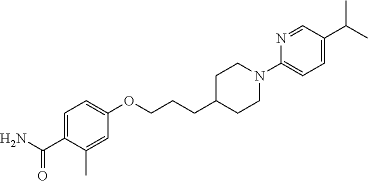 | 4-[3-(5′-Isopropyl-3,4,5,6- tetrahydro-2H- [1,2′]bipyridinyl-4-yl)- propoxy]-2-methyl- benzamide | RT = 2.72 min; m/z (ES⁺) = 396.20 [M + H]⁺ |
| 28 | 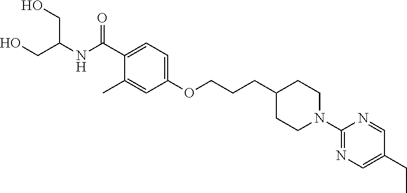 | 4-{3-[1-(5-Ethylpyrimidin- 2-yl)piperidin-4-yl]- propoxy}-N-(2-hydroxy-1- hydroxymethylethyl)-2- methylbenzamide | RT = 3.07 min; m/z (ES⁺) = 457.24 [M + H]⁺ |
| 29 | 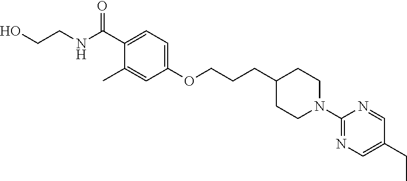 | 4-{3-[1-(5-Ethylpyrimidin- 2-yl)piperidin-4-yl]- propoxy}-N-(2-hydroxy- ethyl)-2-methylbenzamide | RT = 3.20 min; m/z (ES⁺) = 427.36 [M + H]⁺ |
| 30 | 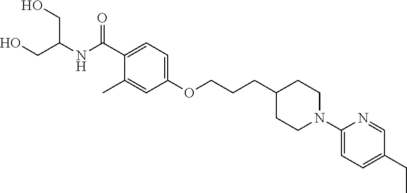 | 4-[3-(5′-Ethyl-3,4,5,6- tetrahydro-2H- [1,2′]bipyridinyl-4-yl)- propoxy]-N-(2-hydroxy-1- hydroxymethylethyl)-2- methylbenzamide | RT = 2.57 min; m/z (ES⁺) = 456.25 [M + H]⁺ |
| 31 | 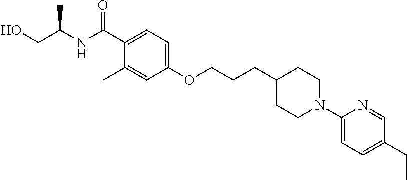 | 4-[3-(5′-Ethyl-3,4,5,6- tetrahydro-2H- [1,2′]bipyridinyl-4-yl)- propoxy]-N-((R)-2-hydroxy- 1-methylethyl)-2-methyl- benzamide | RT = 2.67 min; m/z (ES⁺) = 440.23 [M + H]⁺ |
| 32 | 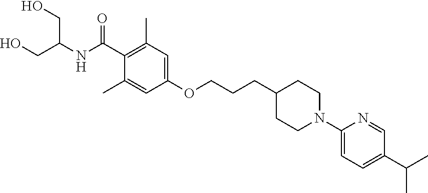 | N-(2-Hydroxy-1- hydroxymethylethyl)-4-[3- (5′-isopropyl-3,4,5,6- tetrahydro-2H- [1,2′]bipyridinyl-4-yl)- propoxy]-2,6-dimethyl- benzamide | RT = 2.65 min; m/z (ES⁺) = 484.26 [M + H]⁺ |
| 33 | 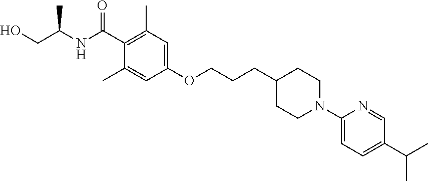 | N-((R)-2-Hydroxy-1-methyl- ethyl)-4-[3-(5′-isopropyl- 3,4,5,6-tetrahydro-2H- [1,2′]bipyridinyl-4-yl)- propoxy]-2,6-dimethyl- benzamide | RT = 2.77 min; m/z (ES⁺) = 468.26 [M + H]⁺ |
| 34 | 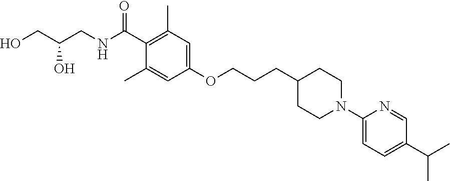 | N-((S)-2,3-Dihydroxy- propyl)-4-[3-(5′-isopropyl- 3,4,5,6-tetrahydro-2H- [1,2′]bipyridinyl-4-yl)- propoxy]-2,6-dimethyl- benzamide | RT = 2.72 min; m/z (ES⁺) = 484.26 [M + H]⁺ |
| 35 | 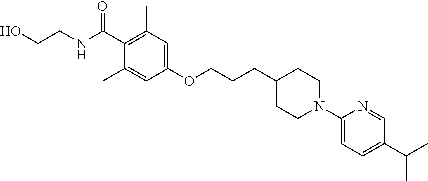 | N-(2-Hydroxyethyl)-4-[3- (5′-isopropyl-3,4,5,6- tetrahydro-2H- [l,2′]bipyridinyl-4-yl)- propoxy]-2,6-dimethyl- benzamide | RT = 2.78 min; m/z (ES⁺) = 454.24 [M + H]⁺ |
| 36 | 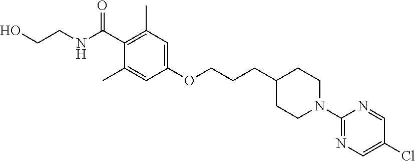 | 4-{3-[1-(5-Chloro- pyrimidin-2-yl)piperidin-4- yl]propoxy}-N-(2-hydroxy- ethyl)-2,6-dimethyl- benzamide | RT = 3.75 min; m/z (ES⁺) = 447.19 [M + H]⁺ |
| 37 | 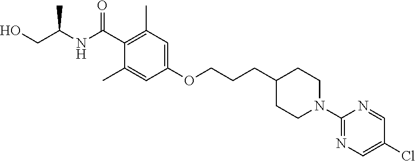 | 4-{3-[1-(5-Chloro- pyrimidin-2-yl)piperidin-4- yl]propoxy}-N-((R)-2- hydroxy-1-methylethyl)-2,6- dimethylbenzamide | RT = 3.80 min; m/z (ES⁺) = 461.18 [M + H]⁺ |
| 38 | 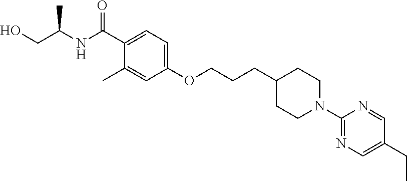 | 4-{3-[1-(5-Ethylpyrimidin- 2-yl)piperidin-4-yl]- propoxy}-N-((R)-2-hydroxy- 1-methylethyl)-2-methyl- benzamide | RT = 3.25 min; m/z (ES⁺) = 441.22 [M + H]⁺ |
| 39 | 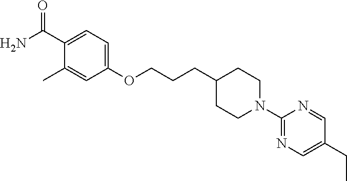 | 4-{3-[1-(5-Ethylpyrimidin- 2-yl)piperidin-4-yl]- propoxy}-2-methyl- benzamide | RT = 3.30 min; m/z (ES⁺) = 383.17 [M + H]⁺ |
| 40 | 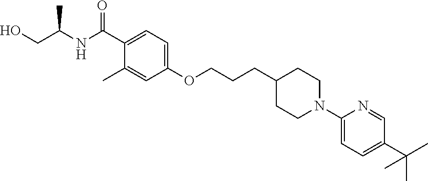 | 4-[3-(5′-tert-Butyl-3,4,5,6- tetrahydro-2H- [1,2′]bipyridinyl-4-yl)- propoxy]-N-((R)-2-hydroxy- 1-methylethyl)-2-methyl- benzamide | RT = 2.84 min; m/z (ES⁺) = 468.27 [M + H]⁺ |
| 41 | 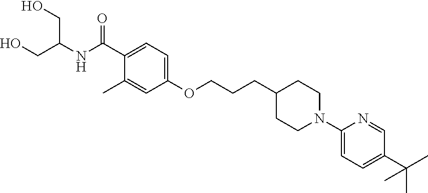 | 4-[3-(5′-tert-Butyl-3,4,5,6- tetrahydro-2H- [1,2′]bipyridinyl-4-yl)- propoxy]-N-(2-hydroxy-1- hydroxymethylethyl)-2- methylbenzamide | RT = 2.73 min; m/z (ES⁺) = 484.26 [M + H]⁺ |
| 42 | 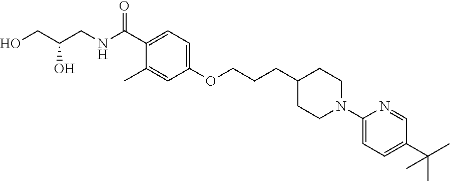 | 4-[3-(5′-tert-Butyl-3,4,5,6- tetrahydro-2H- [1,2′]bipyridinyl-4-yl)- propoxy]-N-((S)-2,3- dihydroxypropyl)-2-methyl- benzamide | RT = 2.82 min; m/z (ES⁺) = 484.29 [M + H]⁺ |
| 43 | 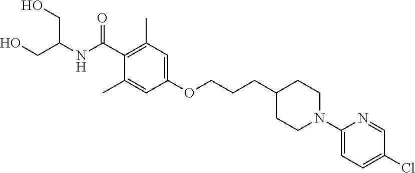 | 4-[3-(5′-Chloro-3,4,5,6- tetrahydro-2H- [1,2′]bipyridinyl-4-yl)- propoxy]-N-(2-hydroxy-1- hydroxymethylethyl)-2,6- dimethylbenzamide | RT = 3.25 min; m/z (ES⁺) = 476.17 [M + H]⁺ |
| 44 | 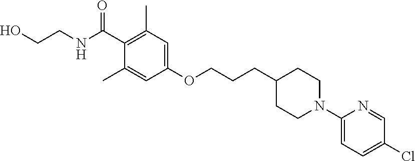 | 4-[3-(5′-Chloro-3,4,5,6- tetrahydro-2H- [1,2′]bipyridinyl-4-yl)- propoxy]-N-(2-hydroxy- ethyl)-2,6-dimethyl- benzamide | RT = 3.40 min; m/z (ES⁺) = 446.15 [M + H]⁺ |
| 45 | 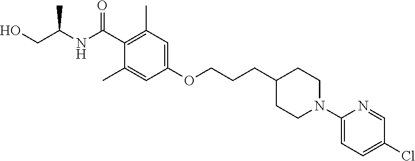 | 4-[3-(5′-Chloro-3,4,5,6- tetrahydro-2H- [1,2′]bipyridinyl-4-yl)- propoxy]-N-((R)-2-hydroxy- 1-methylethyl)-2,6- dimethylbenzamide | RT = 3.52 min; m/z (ES⁺) = 460.17 [M + H]⁺ |
| 46 | 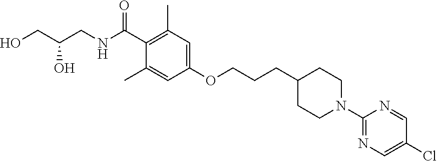 | 4-{3-[1-(5-Chloro- pyrimidin-2-yl)piperidin-4- yl]propoxy}-N-((S)-2,3- dihydroxypropyl)-2,6- dimethylbenzamide | δ_{H} (CDCl₃) 1.12-1.26 (m, 2H), 1.38-1.48 (m, 2H), 1.53-1.64 (m, 1H), 1.76-1.87 (m, 4H), 2.31 (s, 6H), 2.70- 2.77 (m, 2H), 2.82-2.93 (m, 2H), 2.94-2.99 (m, 1H), 3.51-3.76 (m, 4H), 3.86-3.98 (m, 3H), 4.64-4.73 (m, 2H), 6.56 (s, 2H), 8.21 (s, 2H); RT = 3.60 min; m/z (ES⁺) = 477.18 [M + H]⁺ |
| 47 | 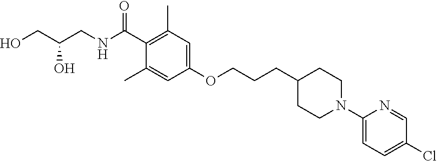 | 4-[3-(5′-Chloro-3,4,5,6- tetrahydro-2H- [1,2′]bipyridinyl-4-yl)- propoxy]-N-((S)-2,3- dihydroxypropyl)-2,6- dimethylbenzamide | δ_{H} (CDCl₃) 1.18-1.32 (m, 2H), 1.37-1.47 (m, 2H), 1.48-1.61 (m, 1H), 1.76-1.88 (m, 4H), 2.32 (s, 6H), 2.71- 2.77 (m, 1H), 2.77-2.88 (m, 2H), 2.95-3.00 (m, 1H), 3.51-3.76 (m, 4H), 3.84-3.98 (m, 3H), 4.19-4.29 (m, 2H), 6.00-6.10 (m, 1H), 6.56 (s, 2H), 6.60 (d, 1H), 7.36-7.43 (m, 1H), 8.08-8.12 (m, 1H); RT = 3.18 min; m/z (ES⁺) = 476.15 [M + H]⁺ |
| 48 | 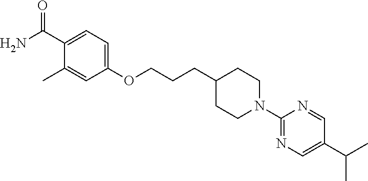 | 4-{3-[1-(5-Isopropyl- pyrimidin-2-yl)piperidin-4- yl]propoxy}-2-methyl- benzamide | RT = 3.55 min; m/z (ES⁺) = 397.26 [M + H]⁺ |
| 49 | 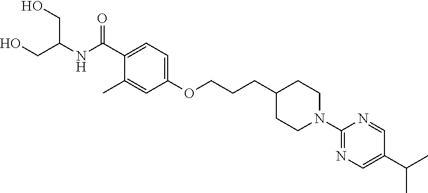 | N-(2-Hydroxy-1- hydroxymethylethyl)-4-{3- [1-(5-isopropylpyrimidin-2- yl)piperidin-4-yl]propoxy}- 2-methylbenzamide | δ_{H} (CDCl₃) 1.14-1.29 (m, 8H), 1.39-1.49 (m, 2H), 1.51-1.63 (m, 1H), 1.75-1.90 (m, 4H), 2.48 (s, 3H), 2.52- 2.59 (m, 2H), 2.77 (sep, 1H), 2.81-2.91 (m, 2H), 3.86-4.02 (m, 6H), 4.10-4.19 (m, 1H), 4.66-4.76 (m, 2H), 6.46-6.55 (m, 1H), 6.68-6.78 (m, 2H), 7.40 (d, 1H), 8.20 (s, 2H); RT = 3.17 min; m/z (ES⁺) = 471.18 [M + H]⁺ |
| 50 | 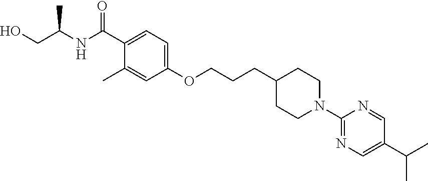 | N-((R)-2-Hydroxy-1-methyl- ethyl)-4-{3-[1-(5-isopropyl- pyrimidin-2-yl)piperidin-4- yl]propoxy}-2-methyl- benzamide | RT = 3.43 min; m/z (ES⁺) = 455.18 [M + H]⁺ |
| 51 | 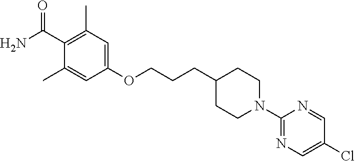 | 4-{3-[1-(5-Chloro- pyrimidin-2-yl)piperidin-4- yl]propoxy}-2,6-dimethyl- benzamide | RT = 3.90 min; m/z (ES⁺) = 403.17 [M + H]⁺ |
| 52 | 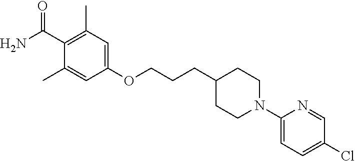 | 4-[3-(5′-Chloro-3,4,5,6- tetrahydro-2H- [1,2′]bipyridinyl-4-yl)- propoxy]-2,6-dimethyl- benzamide | RT = 3.59 min; m/z (ES⁺) = 402.11 [M + H]⁺ |
| 53 | 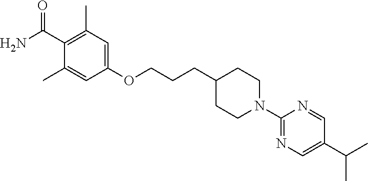 | 4-{3-[1-(5-Isopropyl- pyrimidin-2-yl)piperidin-4- yl]propoxy}-2,6-dimethyl- benzamide | RT = 3.78 min; m/z (ES⁺) = 411.20 [M + H]⁺ |
| 54 | 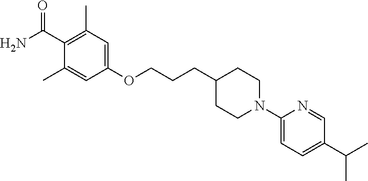 | 4-[3-(5′-Isopropyl-3,4,5,6- tetrahydro-2H- [1,2′]bipyridinyl-4-yl)- propoxy]-2,6-dimethyl- benzamide | RT = 3.42 min; m/z (ES⁺) = 410.20 [M + H]⁺ |
| 55 | 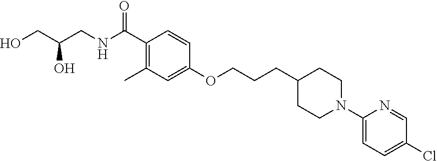 | 4-[3-(5′-Chloro-3,4,5,6- tetrahydro-2H- [1,2′]bipyridinyl-4-yl)- propoxy]-N-((R)-2,3- dihydroxypropyl)-2-methyl- benzamide | RT = 3.29 min; m/z (ES⁺) = 462.17 [M + H]⁺ |
| 56 | 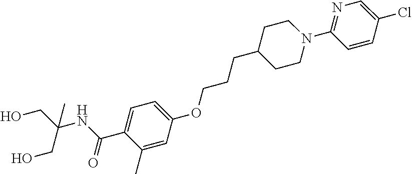 | 4-[3-(5′-Chloro-3,4,5,6- tetrahydro-2H- [1,2′]bipyridinyl-4-yl)- propoxy]-N-(2-hydroxy-1- hydroxymethyl-1-methyl- ethyl)-2-methylbenzamide | RT = 3.50 min; m/z (ES⁺) = 476.20 [M + H]⁺ |
| 57 | 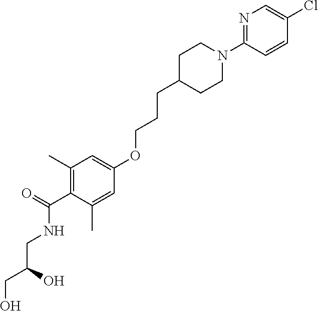 | 4-[3-(5′-Chloro-3,4,5,6- tetrahydro-2H- [1,2′]bipyridinyl-4-yl)- propoxy]-N-((R)-2,3- dihydroxypropyl)-2,6- dimethylbenzamide | RT = 3.18 min; m/z (ES⁺) = 476.21 [M + H]⁺ |
| 58 | 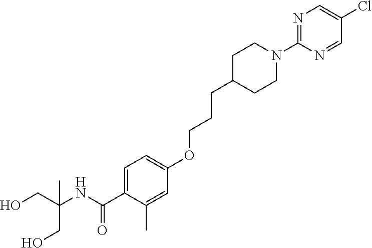 | 4-{3-[1-(5-Chloro- pyrimidin-2-yl)piperidin-4- yl]propoxy}-N-(2-hydroxy- 1-hydroxymethyl-1-methyl- ethyl)-2-methylbenzamide | RT = 3.95 min; m/z (ES⁺) = 477.20 [M + H]⁺ |
| 59 | 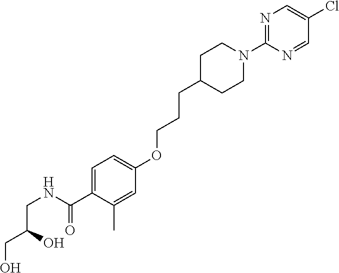 | 4-{3-[1-(5-Chloro- pyrimidin-2-yl)piperidin-4- yl]propoxy}-N-((R)-2,3- dihydroxypropyl)-2-methyl- benzamide | RT = 3.63 min; m/z (ES⁺) = 463.19 [M + H]⁺ |
| 60 | 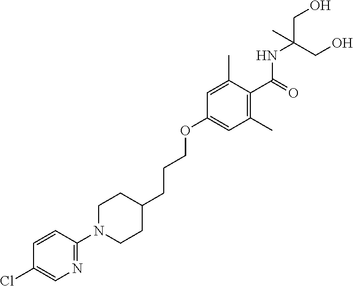 | 4-[3-(5′-Chloro-3,4,5,6- tetrahydro-2H- [1,2′]bipyridinyl-4-yl)- propoxy]-N-(2-hydroxy-1- hydroxymethyl-1-methyl- ethyl)-2,6-dimethyl- benzamide | RT = 3.55 min; m/z (ES⁺) = 490.22 [M + H]⁺ |
| 61 | 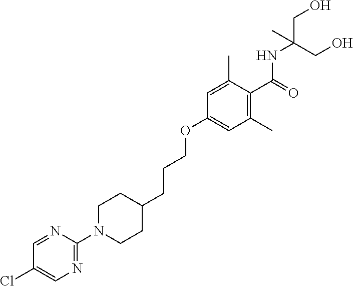 | 4-{3-[1-(5-Chloro- pyrimidin-2-yl)piperidin-4- yl]propoxy}-N-(2-hydroxy- 1-hydroxymethyl-1-methyl- ethyl)-2,6-dimethyl- benzamide | RT = 3.98 min; m/z (ES⁺) = 491.21 [M + H]⁺ |
| 62 | 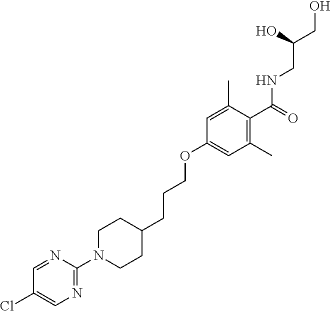 | 4-{3-[1-(5-Chloro- pyrimidin-2-yl)piperidin-4- yl]propoxy}-N-((R)-2,3- dihydroxypropyl)-2,6- dimethylbenzamide | RT = 3.68 min; m/z (ES⁺) = 477.20 [M + H]⁺ |
| 63 | 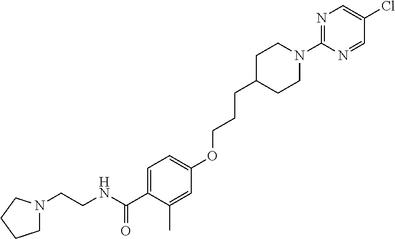 | 4-{3-[1-(5-Chloro- pyrimidin-2-yl)piperidin-4- yl]propoxy}-2-methyl-N-(2- pyrrolidin-1-ylethyl)- benzamide | RT = 3.09 min; m/z (ES⁺) = 486.24 [M + H]⁺ |
| 64 | 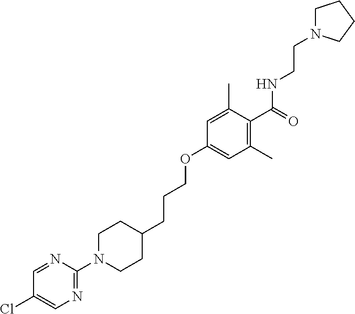 | 4-{3-[1-(5-Chloro- pyrimidin-2-yl)piperidin-4- yl]propoxy}-2,6-dimethyl- N-(2-pyrrolidin-1-ylethyl)- benzamide | RT = 3.04 min; m/z (ES⁺) = 500.25 [M + H]⁺ |
| 65 | 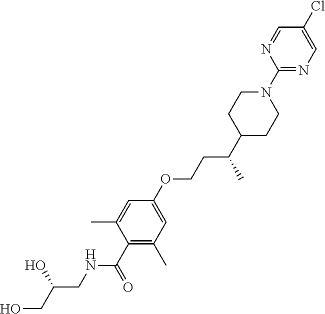 | 4-{(R)-3-[1-(5-Chloro- pyrimidin-2-yl)piperidin-4- yl]butoxy}-N-((R)-2,3- dihydroxypropyl)-2,6- dimethylbenzamide | RT = 3.85 min; m/z (ES⁺) = 491.21 [M + H]⁺ |
| 66 | 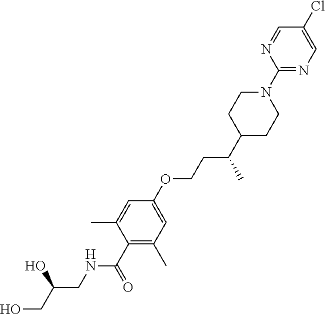 | 4-{(R)-3-[1-(5-Chloro- pyrimidin-2-yl)piperidin-4- yl]butoxy}-N-((S)-2,3- dihydroxypropyl)-2,6- dimethylbenzamide | RT = 3.84 min; m/z (ES⁺) = 491.21 [M + H]⁺ |
| 67 | 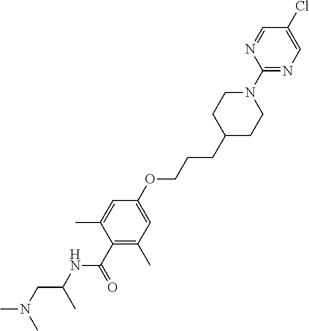 | 4-{3-[1-(5-Chloro- pyrimidin-2-yl)piperidin-4- yl]propoxy}-N-(2- dimethylamino-1-methyl- ethyl)-2,6-dimethyl- benzamide | RT = 3.09 min; m/z (ES⁺) = 488.23 [M + H]⁺ |
| 68 | 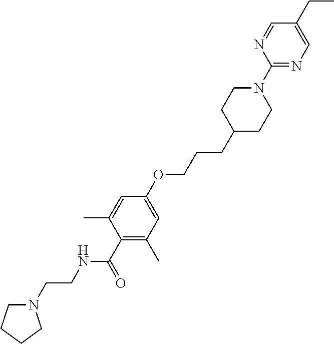 | 4-{3-[1-(5-Ethylpyrimidin- 2-yl)piperidin-4-yl]- propoxy}-2,6-dimethyl-N- (2-pyrrolidin-1-ylethyl)- benzamide | RT = 2.70 min; m/z (ES⁺) = 494.35 [M + H]⁺ |
| 69 | 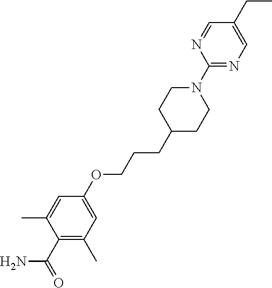 | 4-{3-[1-(5-Ethylpyrimidin- 2-yl)piperidin-4-yl]- propoxy}-2,6-dimethyl- benzamide | RT = 3.38 min; m/z (ES⁺) = 397.24 [M + H]⁺ |
| 70 | 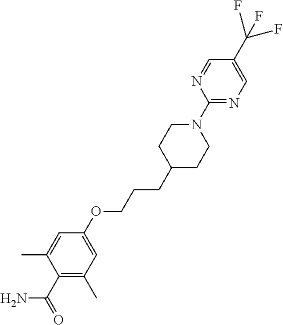 | 2,6-Dimethyl-4-{3-[1-(5- trifluoromethylpyrimidin-2- yl)piperidin-4-yl]propoxy}- benzamide | RT = 4.15 min; m/z (ES⁺) = 437.21 [M + H]⁺ |

### Example 71

### N—((R)-2-Hydroxy-1-methylethyl)-4-{3-[1-(5-isopropylpyrimidin-2-yl)piperidin-4-yl]propoxy}-2,6-dimethylbenzamide

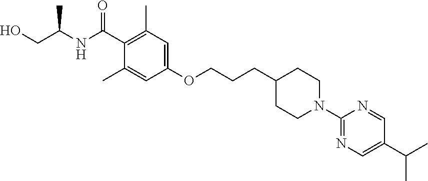

HOAt (70.0 mg, 504 μmol) was added to a stirred solution of 4-{3-[1-(5-isopropylpyrimidin-2-yl)piperidin-4-yl]propoxy}-2,6-dimethylbenzoic acid (Preparation 22, 150 mg, 360 μmol), EDCI (70.5 mg, 370 μmol) and NEt₃ (150 μL, 1.08 mmol) in THF (5 mL). After 20 min, (R)-2-aminopropan-1-ol (85.0 μL, 1.08 mmol) was added and the resulting mixture was heated at 55° C. for 19 h. The THF was removed in vacuo and the residue partitioned between EtOAc and 2M NaOH. The organic phase was separated and washed with 2M NaOH, 1M HCl and brine, before being dried (MgSO₄). Filtration, solvent evaporation, and trituration with EtOAc afforded the title compound: RT=3.48 min; m/z (ES⁺)=469.22 [M+H]⁺ (Method A).

The amides listed in Table 5 were synthesised by condensing the appropriate acid with the appropriate amine, employing a procedure similar to that outlined in Example 71.

TABLE 5

| | | | Spectra: LCMS |
|---|---|---|---|
| Ex | Structure | Name | Method A |
| 72 | 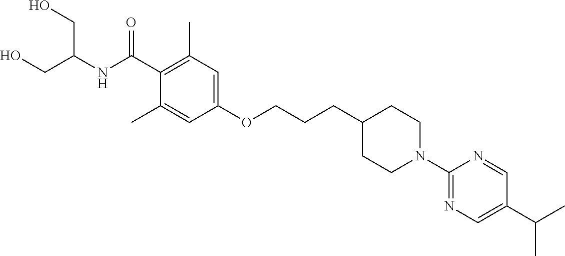 | N-(2-Hydroxy-1- hydroxymethylethyl)-4-{3- [1-(5-isopropylpyrimidin-2- yl)piperidin-4-yl]propoxy}- 2,6-dimethylbenzamide | RT = 3.23 min; m/z (ES⁺) = 485.21 [M + H]⁺ |
| 73 | 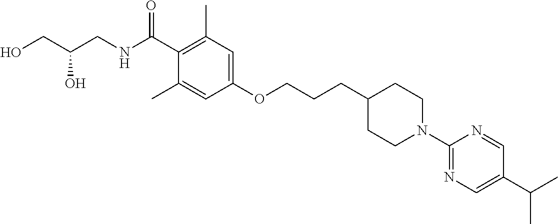 | N-((S)-2,3-Dihydroxy- propyl)-4-{3-[1-(5- isopropylpyrimidin-2-yl)- piperidin-4-yl]propoxy}-2,6- dimethylbenzamide | RT = 3.27 min; m/z (ES⁺) = 485.21 [M + H]⁺ |
| 74 | 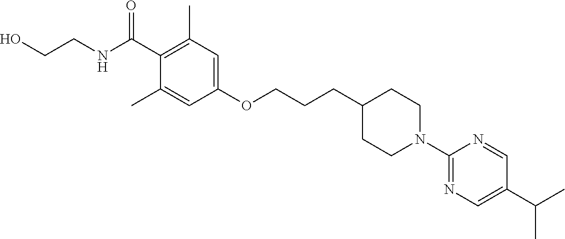 | N-(2-Hydroxyethyl)-4-{3- [1-(5-isopropylpyrimidin-2- yl)piperidin-4-yl]propoxy}- 2,6-dimethylbenzamide | RT = 3.40 min; m/z (ES⁺) = 455.24 [M + H]⁺ |

### Example 75

### N-(2-Aminoethyl)-4-{3-[1-(5-chloropyrimidin-2-yl)piperidin-4-yl]propoxy}-2-methylbenzamide

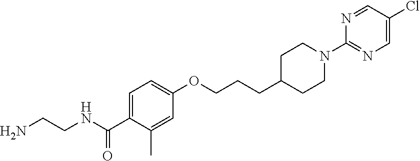

HOBt.H₂O (100 mg, 735 μmol) was added to a stirred solution of 4-{3-[1-(5-chloro-pyrimidin-2-yl)piperidin-4-yl]propoxy}-2-methylbenzoic acid (Preparation 12, 220 mg, 565 mol) and EDCI (141 mg, 735 μmol) in THF (12 mL) and DMF (600 μL). After 15 min, (2-aminoethyl)carbamic acid tert-butyl ester (268 μL, 1.694 mmol) was added and the resulting mixture was stirred at ambient temperature for 96 h. The solvent was removed in vacuo and the residue partitioned between EtOAc and 2M NaOH. The organic phase was separated and washed with 2M NaOH, 2M HCl and brine, before being dried (MgSO₄). Filtration, solvent evaporation, and purification by column chromatography (EtOAc-IH, 1:1 to 1:0) afforded [2-(4-{3-[1-(5-chloropyrimidin-2-yl)piperidin-4-yl]propoxy}-2-methylbenzoylamino)ethyl]carbamic acid tert-butyl ester: RT=4.26 min; m/z (ES⁺)=532.32 [M+H]⁺. To a stirred suspension of this compound in dioxane (7 mL) was added 4M HCl in dioxane (1.75 mL, 7.0 mmol) and the resulting solution was stirred at ambient temperature for 3 h. The solvent was removed in vacuo and the remainder was dissolved EtOAc and 2M HCl. The acidic layer was basified to pH 10 with 2M NaOH and extracted with EtOAc (3×). The combined organic extracts were dried (MgSO₄), filtered and concentrated in vacuo to afford the title compound: RT=3.13 min; ink (ES⁺)=432.21 [M+H]⁺ (Method A).

The amino-containing amides listed in Table 6 were synthesised by condensing the appropriate acid with the appropriate Boc-amino-containing amine, followed by Boc deprotection, employing procedures similar to those outlined in Example 75.

TABLE 6

| Eg | Structure | Name | Spectra: LCMS Method A |
|---|---|---|---|
| 76 | 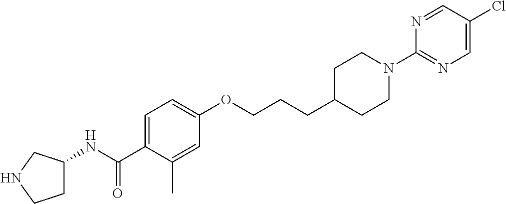 | 4-{3-[1-(5-Chloropyrimidin-2- yl)piperidin-4-yl]propoxy}-2- methyl-N-(R)-pyrrolidin-3-yl- benzamide | RT = 3.09 min; m/z (ES⁺) = 458.14 [M + H]⁺ |
| 77 | 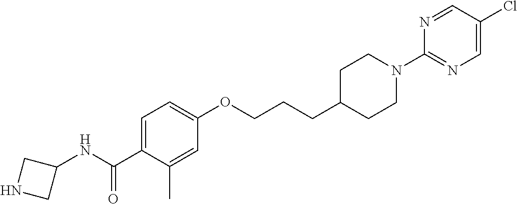 | N-Azetidin-3-yl-4-{3-[1-(5- chloropyrimidin-2-yl)- piperidin-4-yl]propoxy}-2- methylbenzamide | RT = 3.22 min; m/z (ES⁺) = 444.18 [M + H]⁺ |
| 78 | 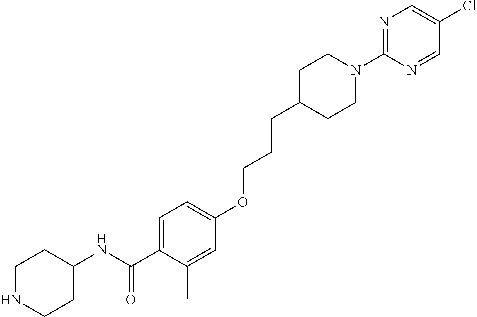 | 4-{3-[1-(5-Chloropyrimidin-2- yl)piperidin-4-yl]propoxy}-2- methyl-N-piperidin-4-yl- benzamide | RT = 2.95 min; m/z (ES⁺) = 472.25 [M + H]⁺ |
| 79 | 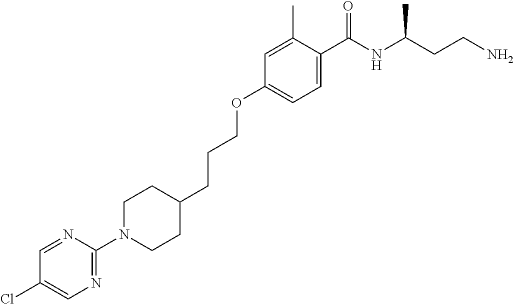 | N-((S)-3-Amino-1-methyl- propyl)-4-{3-[1-(5-chloro- pyrimidin-2-yl)piperidin-4-yl]- propoxy}-2-methylbenzamide | RT = 3.02 min; m/z (ES⁺) = 460.25 [M + H]⁺ |
| 80 | 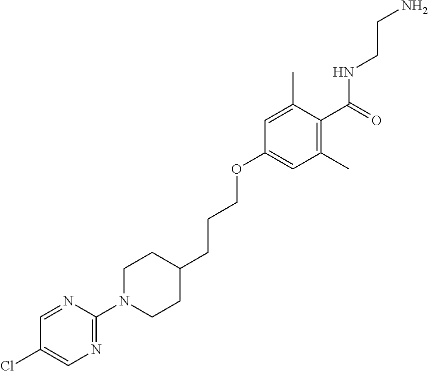 | N-(2-Aminoethyl)-4-{3-[1-(5- chloropyrimidin-2-yl)- piperidin-4-yl]propoxy}-2,6- dimethylbenzamide | RT = 2.98 min; m/z (ES⁺) = 446.23 [M + H]⁺ |
| 81 | 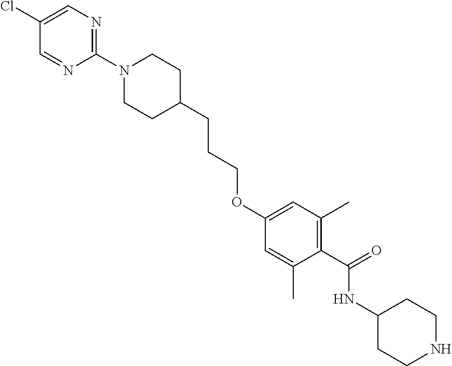 | 4-{3-[1-(5-Chloropyrimidin-2- yl)piperidin-4-yl]propoxy}-2,6- dimethyl-N-piperidin-4-yl- benzamide | RT = 3.20 min; m/z (ES⁺) = 486.27 [M + H]⁺ |
| 82 | 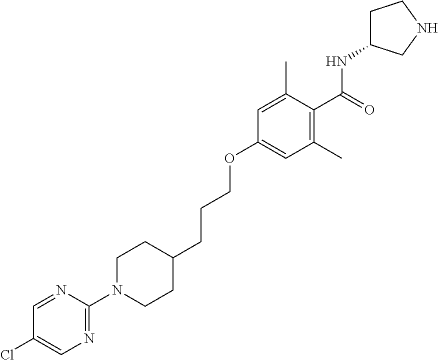 | 4-{3-[1-(5-Chloropyrimidin-2- yl)piperidin-4-yl]propoxy}-2,6- dimethyl-N-(R)-pyrrolidin-3- ylbenzamide | RT = 2.98 min; m/z (ES⁺) = 472.24 [M + H]⁺ |
| 83 | 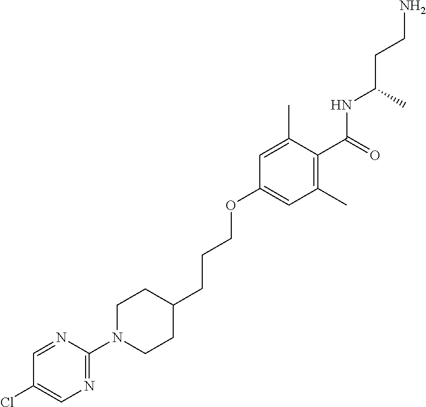 | N-((S)-3-Amino-1-methyl- propyl)-4-{3-[1-(5-chloro- pyrimidin-2-yl)piperidin-4-yl]- propoxy}-2,6-dimethyl- benzamide | RT = 3.15 min; m/z (ES⁺) = 474.26 [M + H]⁺ |
| 84 | 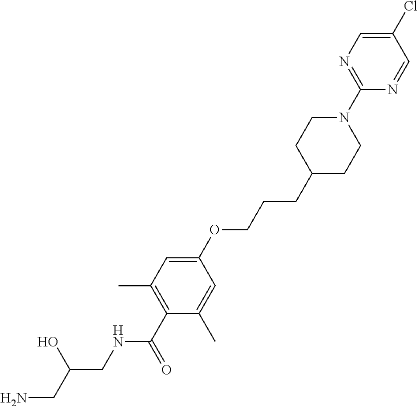 | N-(3-Amino-2-hydroxy- propyl)-4-{3-[1-(5-chloro- pyrimidin-2-yl)piperidin-4- yl]propoxy}-2,6-dimethyl- benzamide | RT = 3.05 min; m/z (ES⁺) = 476.25 [M + H]⁺ |
| 85 | 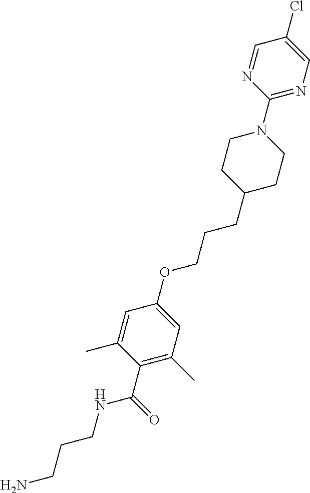 | N-(3-Aminopropyl)-4-{3-[1-(5- chloropyrimidin-2-yl)- piperidin-4-yl]propoxy}-2,6- dimethylbenzamide | RT = 3.17 min; m/z (ES⁺) = 460.25 [M + H]⁺ |
| 86 | 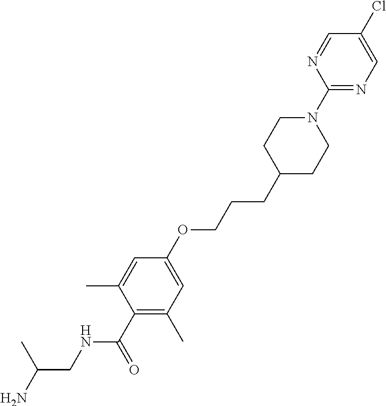 | N-(2-Aminopropyl)-4-{3-[1-(5- chloropyrimidin-2-yl)- piperidin-4-yl]propoxy}-2,6- dimethylbenzamide | RT = 3.18 min; m/z (ES⁺) = 460.25 [M + H]⁺ |
| 87 | 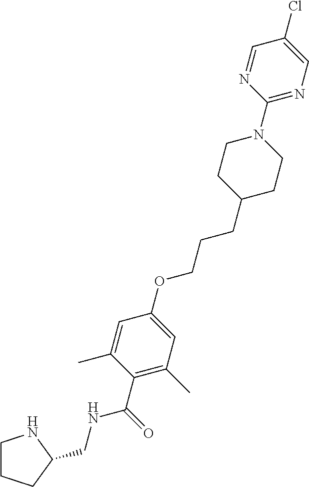 | 4-{3-[1-(5-Chloropyrimidin-2- yl)piperidin-4-yl]propoxy}-2,6- dimethyl-N-(S)-1-pyrrolidin-2- ylmethylbenzamide | RT = 3.27 min; m/z (ES⁺) = 486.26 [M + H]⁺ |
| 88 | 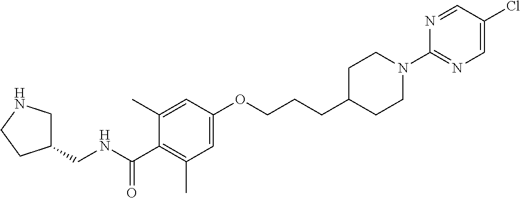 | 4-{3-[1-(5-Chloropyrimidin-2- yl)piperidin-4-yl]propoxy}-2,6- dimethyl-N-(R)-1-pyrrolidin-3- ylmethylbenzamide | RT = 2.97 min; m/z (ES⁺) = 486.27 [M + H]⁺ |
| 89 | 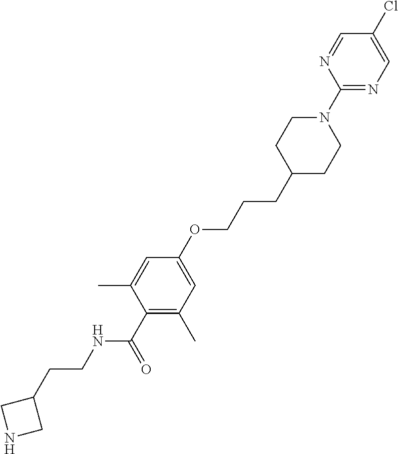 | N-(2-Azetidin-3-ylethyl)-4-{3- [1-(5-chloropyrimidin-2-yl)- piperidin-4-yl]propoxy}-2,6- dimethylbenzamide | RT = 3.00 min; m/z (ES⁺) = 486.27 [M + H]⁺ |
| 90 | 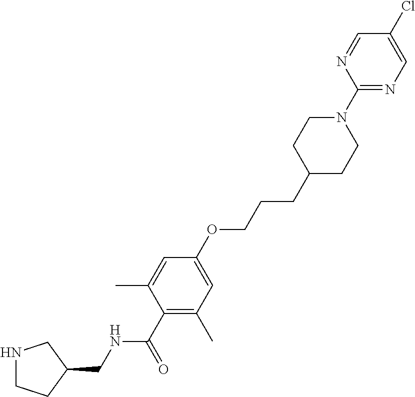 | 4-{3-[1-(5-Chloropyrimidin-2- yl)piperidin-4-yl]propoxy}-2,6- dimethyl-N-(S)-1-pyrrolidin-3- ylmethylbenzamide | RT = 3.02 min; m/z (ES⁺) = 486.27 [M + H]⁺ |
| 91 | 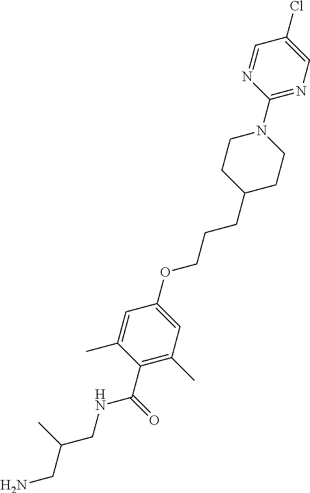 | N-(3-Amino-2-methylpropyl)- 4-{3-[1-(5-chloropyrimidin-2- yl)piperidin-4-yl]propoxy}-2,6- dimethylbenzamide | RT = 3.25 min; m/z (ES⁺) = 454.26 [M + H]⁺ |
| 92 | 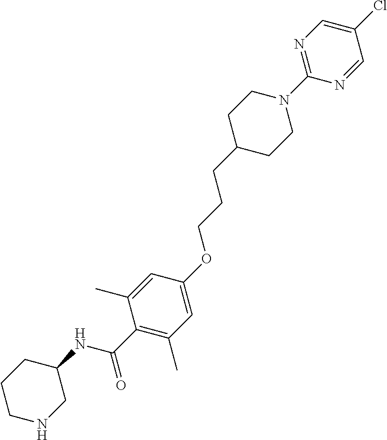 | 4-{3-[1-(5-Chloropyrimidin-2- yl)piperidin-4-yl]propoxy}-2,6- dimethyl-N-(R)-piperidin-3-yl- benzamide | RT = 3.04 min; m/z (ES⁺) = 486.21 [M + H]⁺ |
| 93 | 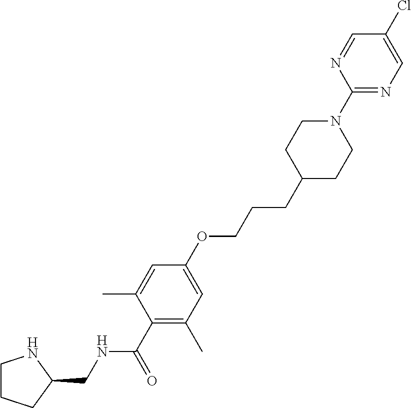 | 4-{3-[1-(5-Chloropyrimidin-2- yl)piperidin-4-yl]propoxy}-2,6- dimethyl-N-(R)-1-pyrrolidin- 2-ylmethylbenzamide | RT = 3.07 min; m/z (ES⁺) = 486.22 [M + H]⁺ |
| 94 | 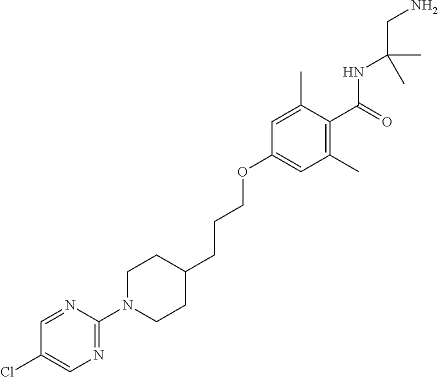 | N-(2-Amino-1,1-dimethyl- ethyl)-4-{3-[1-(5-chloro- pyrimidin-2-yl)piperidin-4-yl]- propoxy}-2,6-dimethyl- benzamide | RT = 3.04 min; m/z (ES⁺) = 474.22 [M + H]⁺ |
| 95 | 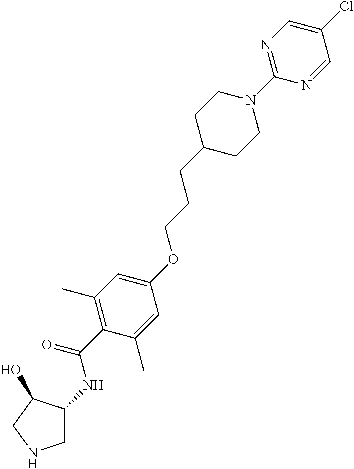 | 4-{3-[1-(5-Chloropyrimidin-2- yl)piperidin-4-yl]propoxy}-N- ((3R,4R)-4-hydroxypyrrolidin- 3-yl)-2,6-dimethylbenzamide | RT = 2.97 min; m/z (ES⁺) = 488.21 [M + H]⁺ |
| 96 | 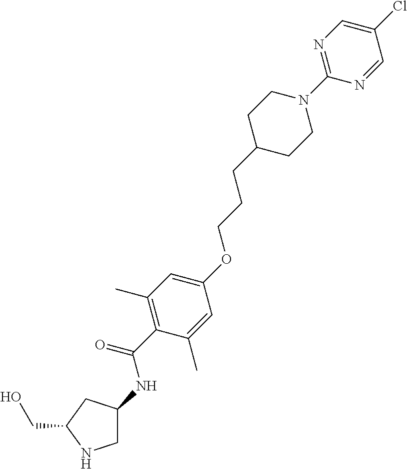 | 4-{3-[1-(5-Chloropyrimidin-2- yl)piperidin-4-yl]propoxy}-N- ((3R,5S)-5-hydroxymethyl- pyrrolidin-3-yl)-2,6-dimethyl- benzamide | RT = 2.95 min; m/z (ES⁺) = 502.22 [M + H]⁺ |
| 97 | 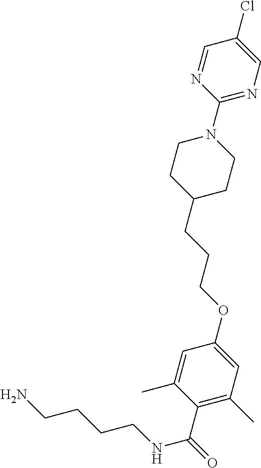 | N-(4-Aminobutyl)-4-{3-[1-(5- chloropyrimidin-2-yl)- piperidin-4-yl]propoxy}-2,6- dimethylbenzamide | RT = 2.92 min; m/z (ES⁺) = 474.23 [M + H]⁺ |
| 98 | 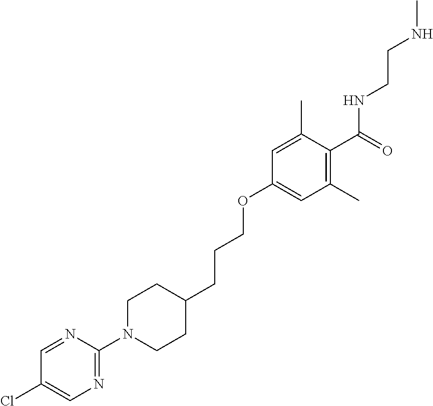 | 4-{3-[1-(5-Chloropyrimidin-2- yl)piperidin-4-yl]propoxy}-2,6- dimethyl-N-(2- methylaminoethyl)benzamide | RT = 2.98 min; m/z (ES⁺) = 460.23 [M + H]⁺ |
| 99 | 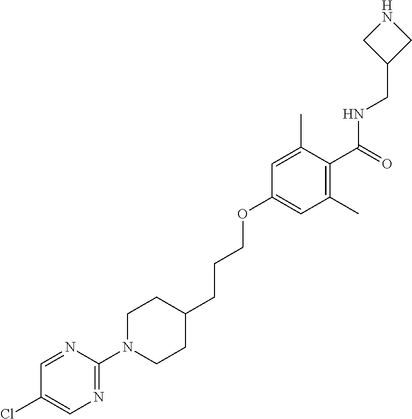 | N-Azetidin-3-ylmethyl-4-{3- [1-(5-chloropyrimidin-2-yl)- piperidin-4-yl]propoxy}-2,6- dimethylbenzamide | RT = 2.97 min; m/z (ES⁺) = 472.22 [M + H]⁺ |
| 100 | 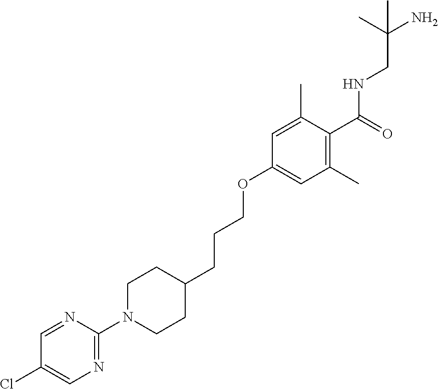 | N-(2-Amino-2-methylpropyl)- 4-{3-[1-(5-chloropyrimidin-2- yl)piperidin-4-yl]propoxy}-2,6- dimethylbenzamide | RT = 3.09 min; m/z (ES⁺) = 474.24 [M + H]⁺ |
| 101 | 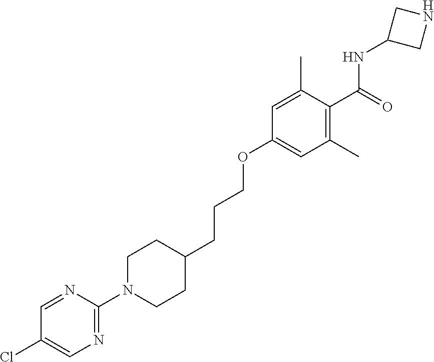 | N-Azetidin-3-yl-4-{3- [1-(5-chloropyrimidin-2-yl)- piperidin-4-yl]propoxy}-2,6- dimethylbenzamide | RT = 2.88 min; m/z (ES⁺) = 458.20 [M + H]⁺ |
| 102 | 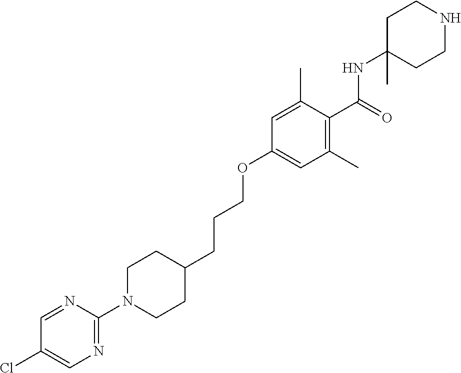 | 4-{3-[1-(5-Chloropyrimidin-2- yl)piperidin-4-yl]propoxy}-2,6- dimethyl-N-(4-methyl- piperidin-4-yl)benzamide | RT = 3.04 min; m/z (ES⁺) = 500.25 [M + H]⁺ |
| 103 | 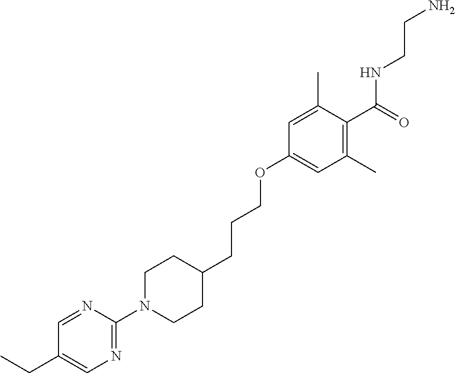 | N-(2-Aminoethyl)-4-{3-[1-(5- ethylpyrimidin-2-yl)piperidin- 4-yl]propoxy}-2,6-dimethyl- benzamide | RT = 2.59 min; m/z (ES⁺) = 440.29 [M + H]⁺ |
| 104 | 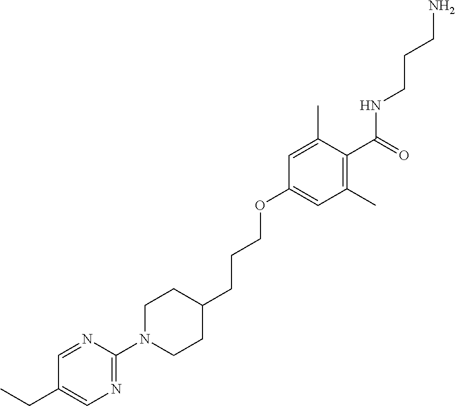 | N-(3-Aminopropyl)-4-{3-[1-(5- ethylpyrimidin-2-yl)piperidin- 4-yl]propoxy}-2,6-dimethyl- benzamide | RT = 2.59 min; m/z (ES⁺) = 454.30 [M + H]⁺ |
| 105 | 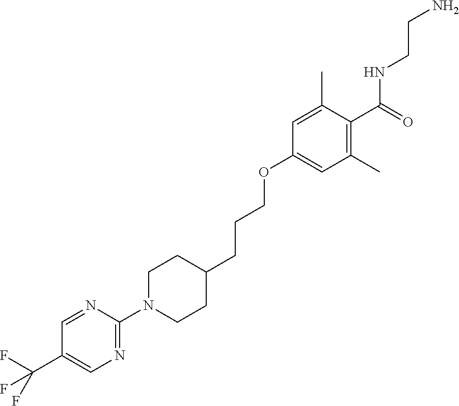 | N-(2-Aminoethyl)-2,6- dimethyl-4-{3-[1-(5- trifluoromethylpyrimidin-2-yl)- piperidin-4-yl]propoxy}- benzamide | RT = 3.02 min; m/z (ES⁺) = 480.23 [M + H]⁺ |
| 106 | 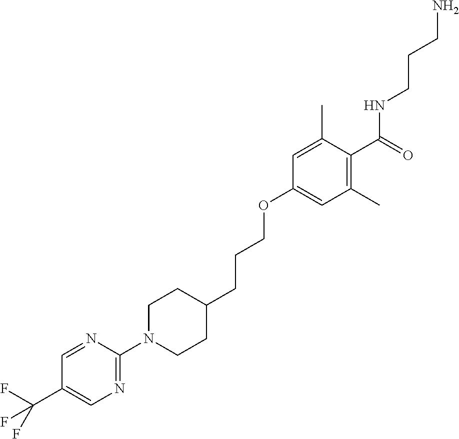 | N-(3-Aminopropyl)-2,6- dimethyl-4-{3-[1-(5- trifluoromethylpyrimidin-2-yl)- piperidin-4-yl]propoxy}- benzamide | RT = 3.02 min; m/z (ES⁺) = 494.24 [M + H]⁺ |
| 107 | 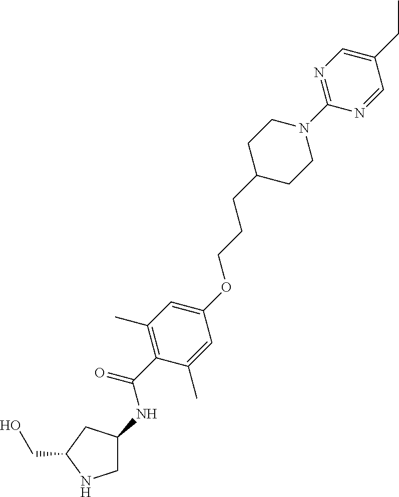 | 4-{3-[1-(5-Ethylpyrimidin-2- yl)piperidin-4-yl]propoxy}-N- ((3R,5S)-5-hydroxymethyl- pyrrolidin-3-yl)-2,6-dimethyl- benzamide | RT = 2.65 min; m/z (ES⁺) = 496.33 [M + H]⁺ |
| 108 | 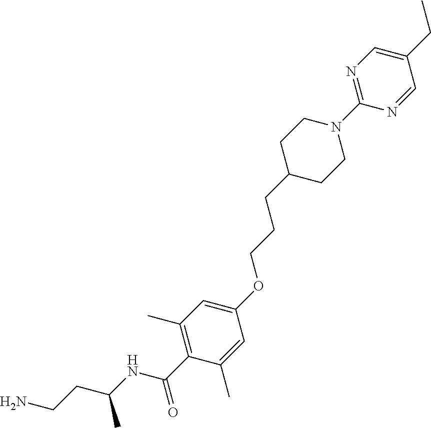 | N-((S)-3-Amino-1-methyl- propyl)-4-{3-[1-(5-ethyl- pyrimidin-2-yl)piperidin-4-yl]- propoxy}-2,6-dimethyl- benzamide | RT = 2.62 min; m/z (ES⁺) = 468.28 [M + H]⁺ |
| 109 | 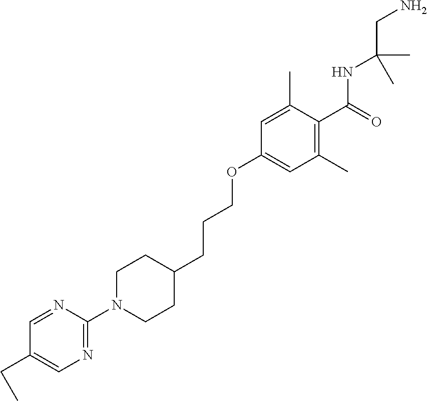 | N-(2-Amino-1,1-dimethyl- ethyl)-4-{3-[1-(5-ethyl- pyrimidin-2-yl)piperidin-4-yl]- propoxy}-2,6-dimethyl- benzamide | RT = 2.77 min; m/z (ES⁺) = 468.32 [M + H]⁺ |

### Examples 110 and 111

### N—((R)-3-Amino-2-hydroxypropyl)-4-{3-[1-(5-chloropyrimidin-2-yl)-piperidin-4-yl]propoxy}-2,6-dimethylbenzamide and N—((S)-3-Amino-2-hydroxypropyl)-4-{3-[1-(5-chloropyrimidin-2-yl)piperidin-4-yl]propoxy}-2,6-dimethylbenzamide

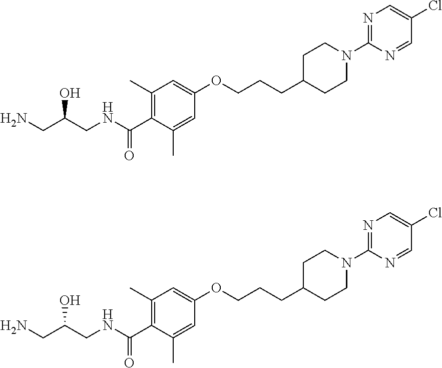

To a solution of 4-{3-[1-(5-chloropyrimidin-2-yl)piperidin-4-yl]propoxy}-2,6-dimethylbenzoic acid (Preparation 20, 602 mg, 1.49 mmol) and (3-amino-2-hydroxypropyl)carbamic acid tert-butyl ester (425 mg, 2.23 mmol) in DMF (15 mL) was added HOBt (245 mg, 1.60 mmol), DIPEA (0.57 mL, 3.27 mmol) and EDCI (350 mg, 1.83 mmol). After stirring at 50° C. for 18 h, the reaction mixture was partitioned between EtOAc (50 mL) and water/brine (150 mL, 1:1). The layers were separated and the aqueous phase extracted with EtOAc (3×50 mL), then the combined organics were washed with 1M HCl (50 mL), 1M NaOH (50 mL) and brine (50 mL). The organic phase was dried (MgSO₄), filtered and concentrated to a residue which was purified by column chromatography (IH:EtOAc, 1:4) to give [3-(4-{3-[1-(5-chloropyrimidin-2-yl)piperidin-4-yl]propoxy}-2,6-dimethylbenzoylamino)-2-hydroxy-propyl]carbamic acid tert-butyl ester as a racemic mixture: RT=4.40 min; m/z (ES⁺)=576.31 [M+H]⁺ (Method A). The individual enantiomers of this compound were separated by preparative chiral HPLC using a Daicel Chiralpak IA column (250×20 mm, 5 μm), with an eluent of MeCN/iPrOH (9:1), at a flow rate of 13 mL/min, and UV detection at 250 nm. To a solution of enantiomer 1 (RT=16.17 min, Daicel Chiralpak IA, 308 mg, 0.535 mmol) in CH₂Cl₂ (8 mL) was added TFA (2 mL) and the solution was stirred at ambient temperature for 3 h. The solvent was removed and the residue was partitioned between EtOAc (200 mL) and 1M NaOH (100 mL). The layers were separated and the aqueous phase extracted with EtOAc (2×50 mL), then the combined organics were washed with brine (100 mL) and dried (MgSO₄). Filtration and removal of the solvent in vacuo provided the free amine, which was redissolved in MeOH (100 mL) and 1M HCl (5 mL). Concentration followed by several co-evaporations with MeOH afforded one of the title compounds as its hydrochloride salt: RT=2.92 min; m/z (ES⁺)=476.21 [M+H]⁺ (Method A). Deprotection of the other enantiomeric Boc-protected intermediate under similar conditions afforded the enantiomeric title compound, which displayed spectroscopic data identical to that described above.

The amides listed in Table 7 were obtained in enantiomerically pure form employing procedures similar to that outlined for Examples 110 and 111, with the exception that the individual enantiomers of the Boc-protected intermediates were separated by preparative chiral HPLC using a Daicel Chiralpak IA column (250×20 mm, 5 μm), with an eluent of IH/iPrOH (7:3) at a flow rate of 15 mL/min, and UV detection at 250 nm.

TABLE 7

| Eg | Structure | Name | Spectra: LCMS Method A |
|---|---|---|---|
| 112 and 113 | 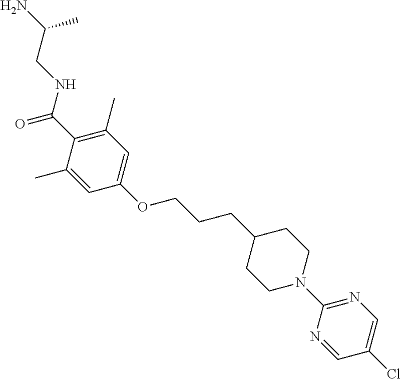 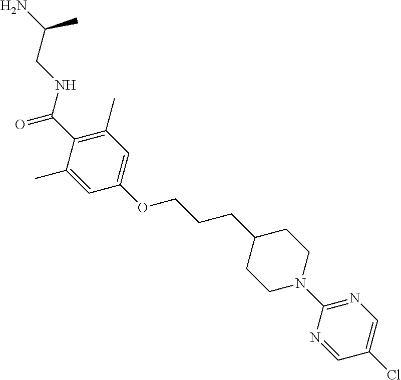 | N-((R)-2-Aminopropyl)-4-{3- [1-(5-chloropyrimidin-2-yl)- piperidin-4-yl]propoxy}-2,6- dimethylbenzamide and N-((S)-2-Aminopropyl)-4-{3- [1-(5-chloropyrimidin-2-yl)- piperidin-4-yl]propoxy}-2,6- dimethylbenzamide | RT = 3.09 min; m/z (ES⁺) = 460.17 [M + H]⁺ |
| 114 and 115 | 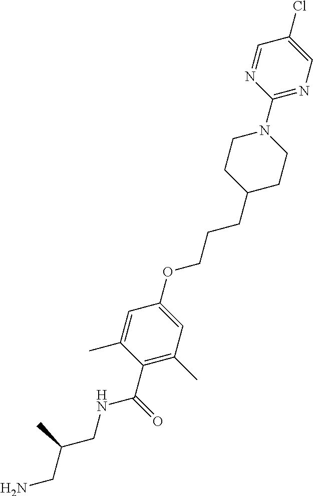 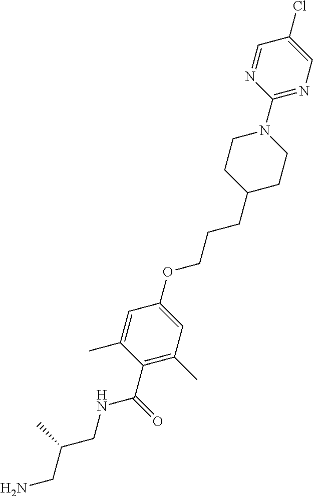 | N-((R)-3-Amino-2-methyl- propyl)-4-{3-[1-(5-chloro- pyrimidin-2-yl)piperidin-4-yl]- propoxy}-2,6-dimethyl- benzamide and N-((S)-3-Amino-2-methyl- propyl)-4-{3-[1-(5-chloro- pyrimidin-2-yl)piperidin-4-yl]- propoxy}-2,6-dimethyl- benzamide | RT = 3.05 min; m/z (ES⁺) = 474.24 [M + H]⁺ |
| 116 and 117 | 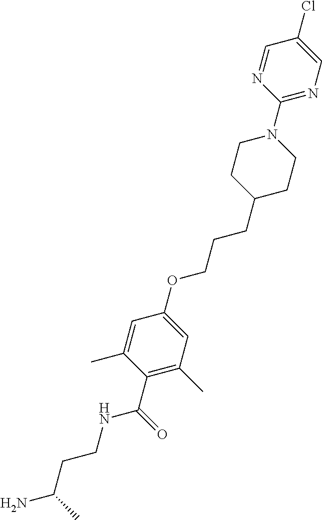 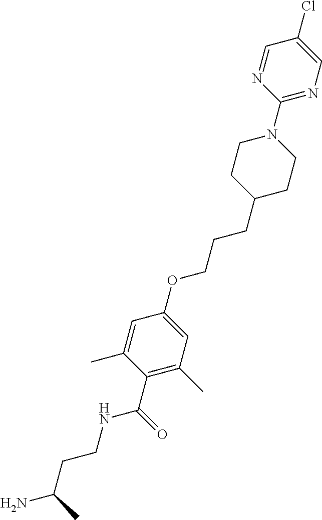 | N-((R)-3-Aminobutyl)-4-{3-[1- (5-chloropyrimidin-2-yl)- piperidin-4-yl]propoxy}-2,6- dimethylbenzamide and N-((S)-3-Aminobutyl)-4-{3-[1- (5-chloropyrimidin-2-yl)- piperidin-4-yl]propoxy}-2,6- dimethylbenzamide | RT = 3.02 min; m/z (ES⁺) = 474.23 [M + H]⁺ |
| 118 and 119 | 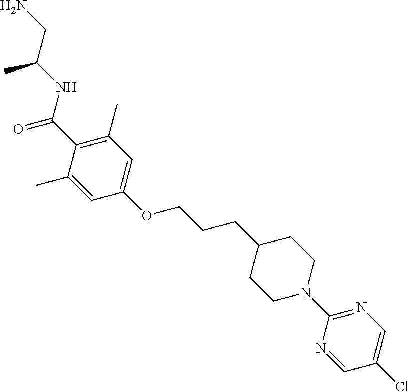 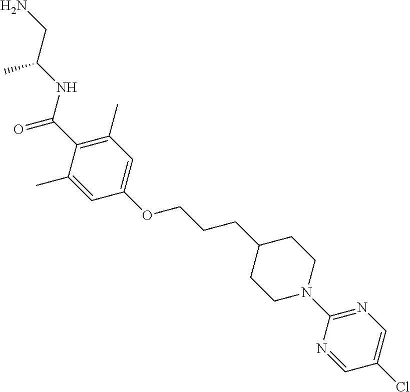 | N-((S)-2-Amino-1-methyl- ethyl)-4-{3-[1-(5-chloro- pyrimidin-2-yl)piperidin-4-yl]- propoxy}-2,6-dimethyl- benzamide and N-((R)-2-Amino-1-methyl- ethyl)-4-{3-[1-(5-chloro- pyrimidin-2-yl)piperidin-4-yl]- propoxy}-2,6-dimethyl- benzamide | RT = 2.95 min; m/z (ES⁺) = 460.23 [M + H]⁺ |
| 120 and 121 | 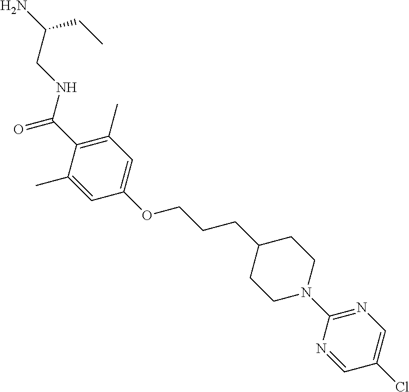 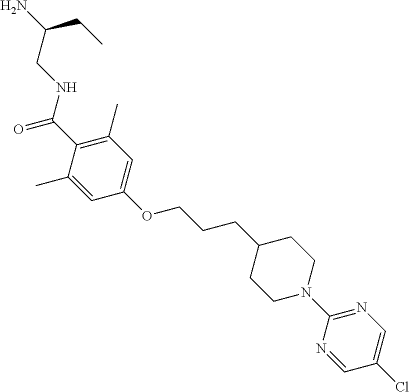 | N-((R)-2-Aminobutyl)-4-{3-[1- (5-chloropyrimidin-2-yl)- piperidin-4-yl]propoxy}-2,6- dimethylbenzamide and N-((S)-2-Aminobutyl)-4-{3-[1- (5-chloropyrimidin-2-yl)- piperidin-4-yl]propoxy}-2,6- dimethylbenzamide | RT = 3.13 min; m/z (ES⁺) = 474.25 [M + H]⁺ |
| 122 and 123 | 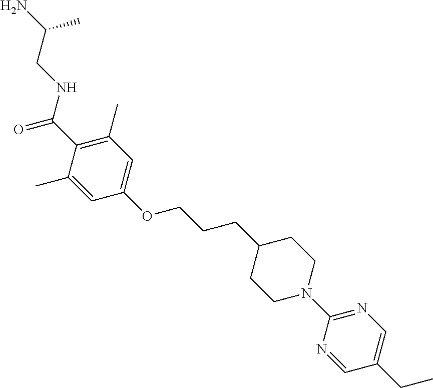 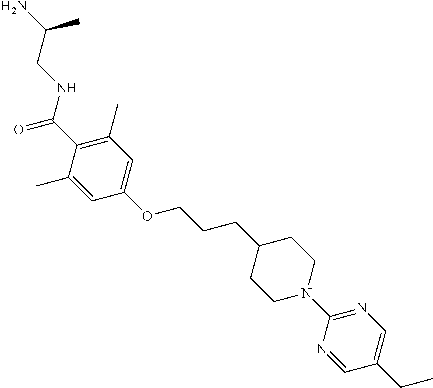 | N-((R)-2-Aminopropyl)-4-{3- [1-(5-ethylpyrimidin-2-yl)- piperidin-4-yl]propoxy}-2,6- dimethylbenzamide and N-((S)-2-Aminopropyl)-4-{3- [1-(5-ethylpyrimidin-2-yl)- piperidin-4-yl]propoxy}-2,6- dimethylbenzamide | RT = 2.59 min; m/z (ES⁺) = 454.30 [M + H]⁺ |
| 124 and 125 | 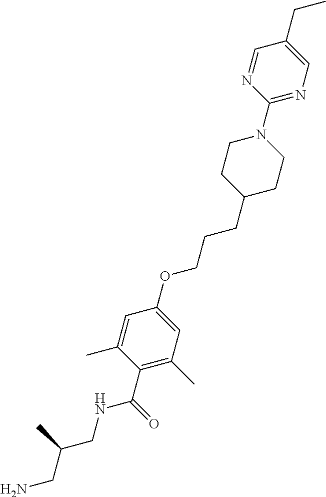 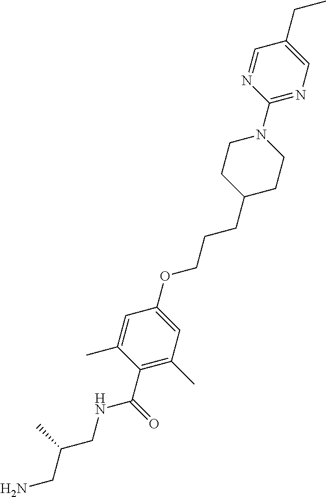 | N-((R)-3-Amino-2-methyl- propyl)-4-{3-[1-(5-ethyl- pyrimidin-2-yl)piperidin-4-yl]- propoxy}-2,6-dimethyl- benzamide and N-((S)-3-Amino-2-methyl- propyl)-4-{3-[1-(5-ethyl- pyrimidin-2-yl)piperidin-4-yl]- propoxy}-2,6-dimethyl- benzamide | RT = 2.70 min; m/z (ES⁺) = 468.33 [M + H]⁺ |
| 126 and 127 | 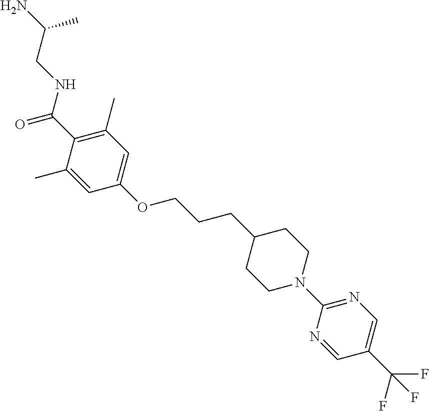 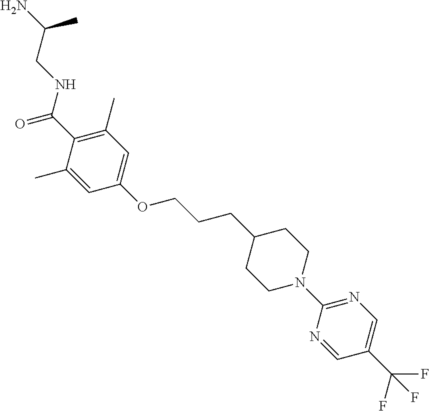 | N-((R)-2-Aminopropyl)-2,6- dimethyl-4-{3-[1-(5- trifluoromethylpyrimidin-2-yl)- piperidin-4-yl]propoxy}- benzamide and N-((S)-2-Aminopropyl)-2,6- dimethyl-4-{3-[1-(5- trifluoromethylpyrimidin-2-yl)- piperidin-4-yl]propoxy}- benzamide | RT = 3.15 min; m/z (ES⁺) = 494.24 [M + H]⁺ |

### Example 128

### 4-[3-(5′-Chloro-3,4,5,6-tetrahydro-2H-[1,2′]bipyridinyl-4-yl)propoxy]-N-(2-hydroxyethyl)-2-methylbenzamide

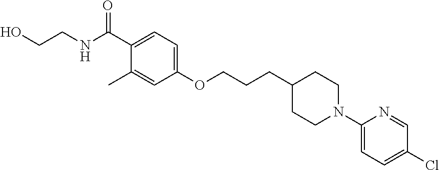

HATU (82.0 mg, 220 μmol) was added to a stirred solution of 4-[3-(5′-chloro-3,4,5,6-tetrahydro-2H-[1,2′]bipyridinyl-4-yl)propoxy]-2-methylbenzoic acid (Preparation 27, 118 mg, 180 μmol), ethanolamine (22.0 μL, 360 μmol) and DIPEA (63.0 μL, 360 μL) in THF (7 mL), and the resulting solution was stirred at ambient temperature for 16 h. The reaction mixture was diluted with DCM (150 mL), washed with H₂O, saturated aqueous NaHCO₃ solution, dried (MgSO₄), filtered and concentrated in vacuo. Purification by RP-HPLC afforded the title compound: RT=3.40 min; m/z (ES⁺)=432.25 [M+H]⁺ (Method A).

The amides listed in Table 8 were synthesised by condensing the appropriate acid with the appropriate amine, employing a procedure similar to that outlined in Example 128.

TABLE 8

| | | | Spectra: LCMS |
|---|---|---|---|
| Ex | Structure | Name | Method A |
| 129 | 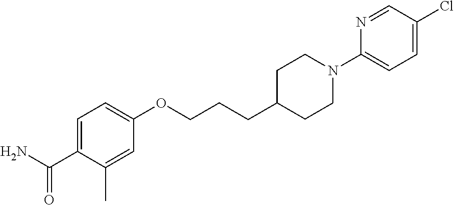 | 4-[3-(5′-Chloro-3,4,5,6- tetrahydro-2H- [1,2′]bipyridinyl-4-yl)- propoxy]-2-methylbenzamide- 2-methylbenzamide | RT = 3.47 min; m/z (ES⁺) = 388.18 [M + H]⁺ |
| 130 | 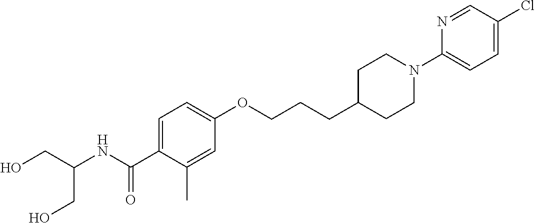 | 4-[3-(5′-Chloro-3,4,5,6- tetrahydro-2H- [1,2′]bipyridinyl-4-yl)- propoxy]-N-(2-hydroxy-1- hydroxymethylethyl)-2-methyl- benzamide | RT = 3.18 min; m/z (ES⁺) = 462.22 [M + H]⁺ |
| 131 | 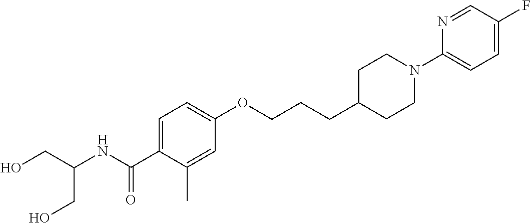 | 4-[3-(5′-Fluoro-3,4,5,6- tetrahydro-2H- [1,2′]bipyridinyl-4-yl)- propoxy]-N-(2-hydroxy-1- hydroxymethylethyl)-2-methyl benzamide | RT = 2.81 min; m/z (ES⁺) = 446.23 [M + H]⁺ |
| 132 | 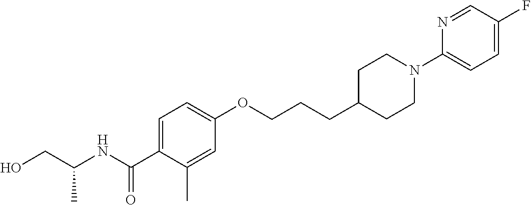 | 4-[3-(5′-Fluoro-3,4,5,6- tetrahydro-2H- [1,2′]bipyridinyl-4-yl)- propoxy]-N-((R)-2-hydroxy-1- methylethyl)-2-methyl- benzamide | RT = 3.03 min; m/z (ES⁺) = 430.28 [M + H]⁺ |
| 133 | 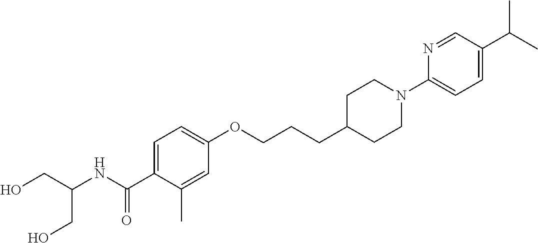 | N-(2-Hydroxy-1- hydroxymethylethyl)-4-[3-(5′- isopropyl-3,4,5,6-tetrahydro- 2H-[1,2′]bipyridinyl-4-yl)- propoxy]-2-methylbenzamide | RT = 2.70 min; m/z (ES⁺) = 470.27 [M + H]⁺ |
| 134 | 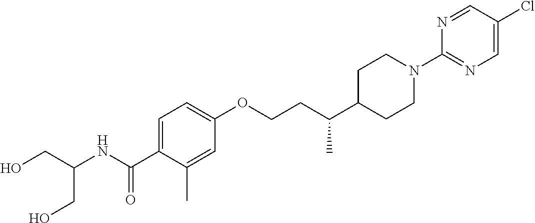 | 4-{(R)-3-[1-(5-Chloro- pyrimidin-2-yl)piperidin-4-yl]- butoxy}-N-(2-hydroxy-1- hydroxymethylethyl)-2-methyl- benzamide | RT = 3.78 min; m/z (ES⁺) = 477.20 [M + H]⁺ |
| 135 | 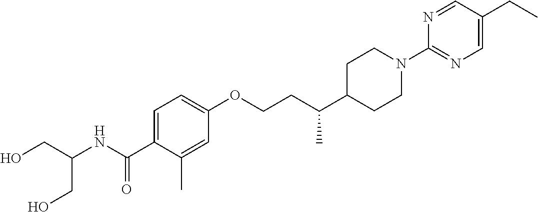 | 4-{(R)-3-[1-(5-Ethyl- pyrimidin-2-yl)piperidin-4-yl]- butoxy}-N-(2-hydroxy-1- hydroxymethylethyl)-2-methyl- benzamide | RT = 3.09 min; m/z (ES⁺) = 471.24 [M + H]⁺ |
| 136 | 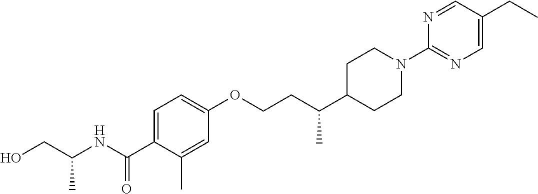 | 4-{(R)-3-[1-(5-Ethyl- pyrimidin-2-yl)-piperidin-4- yl]butoxy}-N-((R)-2-hydroxy- 1-methylethyl)-2-methyl- benzamide | RT = 3.39 min; m/z (ES⁺) = 455.25 [M + H]⁺ |

### Example 137

### N-(2-Hydroxyethyl)-4-[3-(5′-isopropyl-3,4,5,6-tetrahydro-2H-[1,2′]bipyridinyl-4-yl)propoxy]-2-methylbenzamide

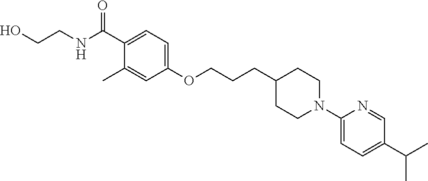

2-Chloro-5-isopropylpyridine (65.0 mg, 420 μmol) was added to a stirred solution of N-(2-hydroxyethyl)-2-methyl-4-(3-piperidin-4-ylpropoxy)benzamide hydrochloride (Preparation 9, 100 mg, 280 μmol) and DBU (63.0 μL, 420 μmol) in DMSO (0.5 mL). The reaction mixture was heated at 125° C. for 60 h, before being poured into H₂O (50 mL) and extracted with EtOAc (3×). The combined organic extracts were washed with brine, dried (MgSO₄), filtered and concentrated in vacuo. Purification by column chromatography (EtOAc) afforded the title compound: RT=2.73 min; m/z (ES⁺)=440.28 [M+H]⁺ (Method A).

The compounds listed in Table 9 were synthesized from N-(2-hydroxyethyl)-2-methyl-4-(3-piperidin-4-ylpropoxy)benzamide hydrochloride (Preparation 9) and 2,5-difluoropyridine or 3,6-dichloropyridazine, employing a procedure similar to that outlined in Example 137.

### Table 9

| | | | Spectra: LCMS |
|---|---|---|---|
| Ex | Structure | Name | Method A |
| 138 | 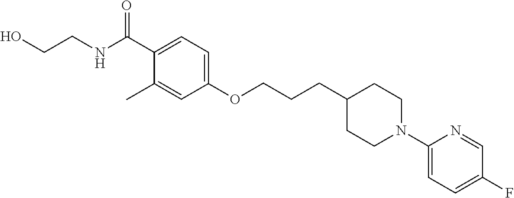 | 4-[3-(5′-Fluoro-3,4,5,6-tetrahydro-2H- [1,2′]bipyridinyl-4-yl)-propoxy]-N- (2-hydroxyethyl)-2-methylbenzamide | RT = 2.98 min; m/z (ES⁺) = 416.21 [M + H]⁺ |
| 139 | 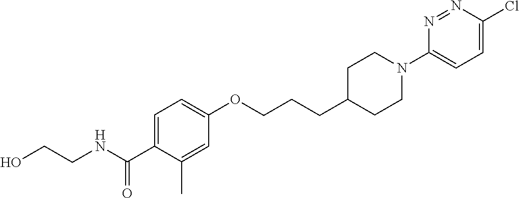 | 4-{3-[1-(6-Chloropyridazin-3- yl)piperidin-4-yl]propoxy}-N- (2-hydroxyethyl)-2-methyl- benzamide | RT = 3.10 min; m/z (ES⁺) = 433.18 [M + H]⁺ |

### Example 140

### N-(2-Hydroxyethyl)-4-{3-[1-(6-isopropoxypyridazin-3-yl)piperidin-4-yl]-propoxy}-2-methylbenzamide

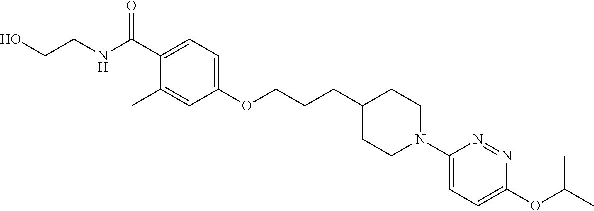

To a solution of N-(2-hydroxyethyl)-2-methyl-4-(3-piperidin-4-ylpropoxy)benzamide hydrochloride (Preparation 9, 50.0 mg, 140 μmol) in dioxane (1.5 mL) was added 3-chloro-6-isopropoxypyridazine (36.0 mg, 210 μmol), NaOt-Bu (27.0 mg, 280 μmol) and 2,8,9-triisobutyl-2,5,8,9-tetraaza-1-phosphabicyclo[3.3.3]undecane (5.0 mg, 14.0 μmol). Argon was bubbled through the reaction mixture for 20 min, then palladium acetate (11.0 mg, 49.0 μmol) was added. Argon was bubbled through the reaction mixture for 10 min, then the reaction mixture was heated in a sealed tube in the microwave at 120° C. for 2 h. The reaction mixture was diluted with methanol, filtered through celite and concentrated in vacuo. Purification by column chromatography (EtOAc) afforded the title compound: RT=2.67 min; m/z (ES)=457.28 [M+H]⁺ (Method A).

### Example 141

### 4-{3-[1-(5-Fluoropyrimidin-2-yl)piperidin-4-yl]propoxy}-N-(2-hydroxyethyl)-2-methylbenzamide

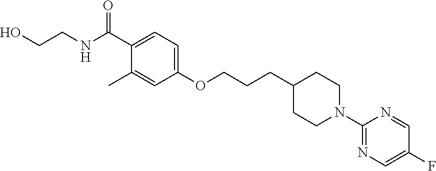

To a solution of N-(2-hydroxyethyl)-2-methyl-4-(3-piperidin-4-ylpropoxy)benzamide hydrochloride (Preparation 9, 68.0 mg, 191 μmol) in toluene (1.5 mL) was added 2-chloro-5-fluoropyrimidine (27.8 mg, 210 μmol), NaOt-Bu (45.9 mg, 478 μmol) and 2,8,9-triisobutyl-2,5,8,9-tetraaza-1-phosphabicyclo[3.3.3]undecane (7.0 mg, 19.1 μmol). Argon was bubbled through the reaction mixture for 15 min, then bis(dibenzlideneacetone)palladium (17.5 mg, 19.1 μmol) was added. Argon was bubbled through the reaction mixture for 15 min, then the reaction mixture was heated in a sealed tube in the microwave at 100° C. for 1 h. The reaction mixture was diluted with methanol, filtered through celite and concentrated in vacuo. The remainder was dissolved in EtOAc, washed with H₂O, dried (MgSO₄), filtered and concentrated in vacuo. Purification by RP-HPLC afforded the title compound: RT=3.53 min; m/z (ES⁺)=417.20 [M+H]⁺ (Method A).

### Example 142

### 4-{3-[1-(5-Chloropyrimidin-2-yl)piperidin-4-yl]propoxy}-N-((1R,2S)-2,3-dihydroxy-1-methylpropyl)-2,6-dimethylbenzamide

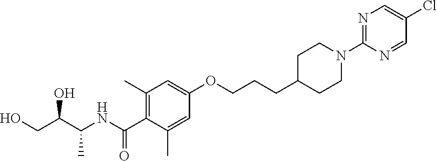

To a solution of 4-{3-[1-(5-chloropyrimidin-2-yl)piperidin-4-yl]propoxy}-2,6-dimethylbenzoic acid (Preparation 20, 168 mg, 0.416 mmol) and (+)-(4S,1′R)-2,2-dimethyl-4-(1′-aminoethyl)-1,3-dioxolane (70 mg, 0.484 mmol) in DMF (10 mL) was added HOBt (65 mg, 0.424 mmol), DIPEA (0.16 mL, 0.92 mmol) and EDCI (98 mg, 0.511 mmol). After stirring at 50° C. for 18 h, the reaction mixture was partitioned between EtOAc (50 mL) and water/brine (150 mL, 1:1). The layers were separated and the aqueous phase extracted with EtOAc (3×50 mL), then the combined organics were washed with 1M NaOH solution (50 mL) and brine (50 mL). The organic phase was dried (MgSO₄), filtered and concentrated to a residue before being redissolved in 1M HCl solution (in MeOH, 50 mL). After removal of the solvent in vacuo the crude material was purified by column chromatography (DCM:MeOH, 10:1) to give the title compound: RT=3.82 min; m/z (ES⁺)=491.18 [M+H]⁺ (Method A).

### Example 143

### 6-{(R)-3-[1-(5-Chloropyrimidin-2-yl)piperidin-4-yl]butoxy}-N—((S)-2,3-dihydroxypropyl)-2-methylnicotinamide

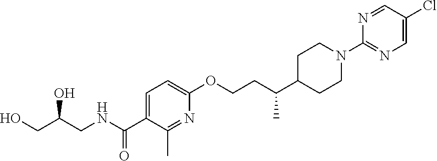

HATU (113 mg, 298 μmol) was added to a stirred solution of 6-{(R)-3-[1-(5-chloro-pyrimidin-2-yl)piperidin-4-yl]butoxy}-2-methylnicotinic acid (Preparation 45, 100 mg, 248 μmol), (S)-3-aminopropane-1,2-diol (34.0 mg, 372 μmol) and DIPEA (65.0 μL, 372 μL) in THF (7 mL), and the resulting solution was stirred at ambient temperature for 72 h. The reaction mixture was diluted with EtOAc and washed with 10% aqueous citric acid, H₂O (3×) and brine, dried (MgSO₄), filtered and concentrated in vacuo. Purification by recrystallisation (EtOAc) afforded the title compound: δ_{H} (CDCl₃) 0.94 (d, 3H), 1.20-1.40 (m, 2H), 1.48-1.67 (m, 3H), 1.68-1.78 (m, 2H), 1.85-1.98 (m, 1H), 2.61 (s, 3H), 2.75-2.98 (m, 4H), 3.52-3.72 (m, 4H), 3.84-3.94 (m, 1H), 4.30-4.45 (m, 2H), 4.70-4.80 (m, 2H), 6.17-6.28 (m, 1H), 6.54 (d, 1H), 7.62 (d, 1H), 8.21 (s, 2H); RT=3.61 min; m/z (ES⁺)=478.15 [M+H]⁺ (Method A).

### Example 144

### 6-{(R)-3-[1-(5-Chloropyrimidin-2-yl)piperidin-4-yl]butoxy}-N-(2-hydroxy-1-hydroxymethylethyl)-2-methylnicotinamide

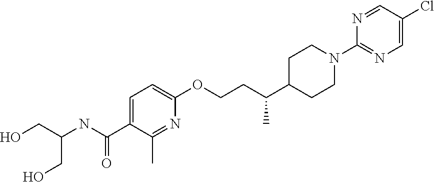

The title compound was synthesized from 6-{(R)-3-[1-(5-chloropyrimidin-2-yl)-piperidin-4-yl]butoxy}-2-methylnicotinic acid (Preparation 45, 100 mg, 248 μmol) and 2-aminopropane-1,3-diol (34 mg, 372 μmol) emplying a procedure similar to that outlined in Example 143: RT=3.51 min; m/z (ES⁺)=478.14 [M+H]⁺ (Method A).

### Example 145

### N—((R)-2-Hydroxy-1-methylethyl)-6-{3-[1-(5-isopropylpyrimidin-2-yl)piperidin-4-yl]propoxy}-2-methylnicotinamide

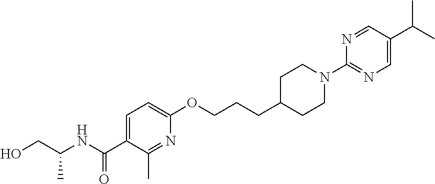

HOBt.H₂O (36.0 mg, 270 μmol) was added to a stirred solution of 6-{3-[1-(5-isopropyl-pyrimidin-2-yl)piperidin-4-yl]propoxy}-2-methylnicotinic acid (Preparation 47, 100 mg, 250 μmol) and EDCI (51.5 mg, 270 μmol) in THF (7 mL). After 20 min, (R)-2-aminopropan-1-ol (20.0 mg, 270 μmol) was added and the resulting mixture was stirred at ambient temperature for 16 h. The THF was removed in vacuo and the residue partitioned between EtOAc and 2M NaOH. The organic phase was separated and washed with 2M NaOH, 1M HCl and brine, before being dried (MgSO₄). Filtration, solvent evaporation, and purification by recrystallisation afforded the title compound: RT=3.79 min; m/z (ES⁺)=433.24 [M+H]⁺ (Method A).

### Example 146

### N-(2-Hydroxy-1-hydroxymethylethyl)-6-{3-[1-(5-isopropylpyrimidin-2-yl)-piperidin-4-yl]propoxy}-2-methylnicotinamide

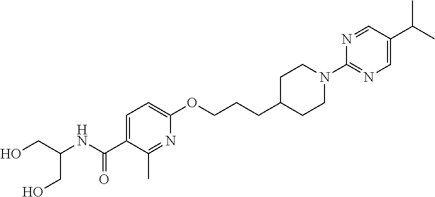

The title compound was synthesized from 6-{3-[1-(5-isopropylpyrimidin-2-yl)-piperidin-4-yl]propoxy}-2-methylnicotinic acid (Preparation 47, 100 mg, 250 μmol) and 2-aminopropane-1,3-diol (25.0 mg, 270 μmol) employing a procedure similar to that outlined in Example 145: RT=2.98 min; m/z (ES⁺)=472.22 [M+H]⁺ (Method A).

### Example 147

### 6-{3-[1-(5-Chloropyrimidin-2-yl)piperidin-4-yl]propoxy}-N-(2-hydroxy-1-hydroxymethylethyl)-2-methylnicotinamide

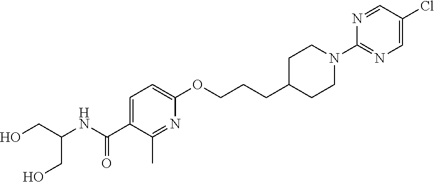

To a solution of N-(2-hydroxy-1-hydroxymethylethyl)-2-methyl-6-(3-piperidin-4-yl-propoxy)nicotinamide hydrochloride (Preparation 50, 125 mg, 320 μmol) in DMSO (5 mL) was added 2,5-dichloropyrimidine (72.0 mg, 490 μmol) and DBU (119 μL, 800 μmol) and the resulting solution was stirred at 100° C. for 16 h. The reaction mixture was diluted with EtOAc (100 mL) then washed with H₂O and brine, before being dried (MgSO₄), filtered and concentrated in vacuo. Purification by recrystallisation (EtOAc) and RP-HPLC afforded the title compound: RT=3.48 min; m/z (ES⁺)=464.16 [M+H]⁺ (Method A).

### Example 148

### 6-[3-(5′-Chloro-3,4,5,6-tetrahydro-2H-[1,2′]bipyridinyl-4-yl)propoxy]-N-(2-hydroxy-1-hydroxymethylethyl)-2-methylnicotinamide

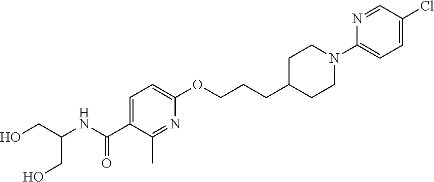

To a solution of N-(2-hydroxy-1-hydroxymethylethyl)-2-methyl-6-(3-piperidin-4-yl-propoxy)nicotinamide hydrochloride (Preparation 50, 125 mg, 320 μmol) in DMSO (5 mL) was added 5-chloro-2-fluoropyridine (70.2 mg, 533 μmol) and DBU (133 μL, 889 μmol) and the resulting solution was stirred at 100° C. for 16 h. Further 5-chloro-2-fluoropyridine (93.7 mg, 712 μmol) was added to the reaction mixture and heating at 70° C. was continued for 72 h. The reaction mixture was diluted with EtOAc, then washed with H₂O and brine, before being dried (MgSO₄), filtered and concentrated in vacuo to afford the title compound: RT=2.90 min; m/z (ES⁺)=463.22 [M+H]⁺ (Method A).

### Example 149

### 6-{3-[1-(5-Ethylpyrimidin-2-yl)piperidin-4-yl]propoxy}-2-methylnicotinamide

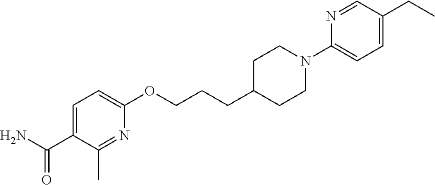

The title compound was synthesized from 6-{3-[1-(5-ethylpyrimidin-2-yl)piperidin-4-yl]propoxy}-2-methylnicotinic acid (Preparation 52, 240 mg, 624 μmol) and 0.5M NH₃ in dioxane (6.24 mL, 3.12 mmol) employing a procedure similar to that outlined in Example 145: RT=3.11 min; m/z (ES⁺)=384.21 [M+H]⁺ (Method A).

The amides listed in Table 10 were synthesised by condensing the appropriate acid with the appropriate amine, employing procedures similar to those outlined in Examples 145 and 149.

TABLE 10

| Ex | Structure | Name | Spectra: LCMS |
|---|---|---|---|
| 150 | 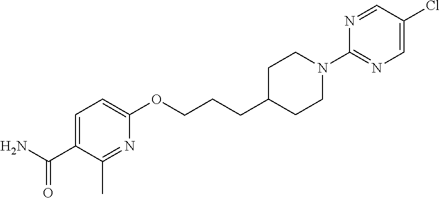 | 6-{3-[1-(5-Chloro- pyrimidin-2-yl)piperidin-4- yl]propoxy}-2-methyl- nicotinamide | RT = 3.88 min; m/z (ES⁺) = 390.12 [M + H]⁺ (Method A) |
| 151 | 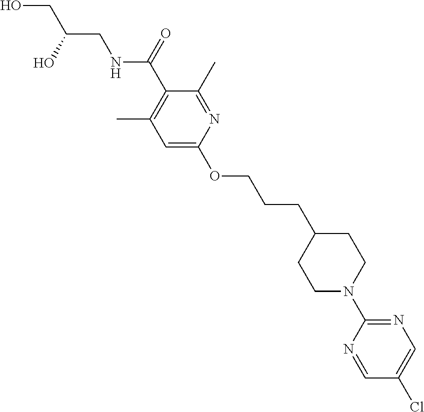 | 6-{3-[1-(5-Chloro- pyrimidin-2-yl)piperidin-4- yl]propoxy}-N-((S)-2,3- dihydroxypropyl)-2,4- dimethylnicotinamide | RT = 3.32 min; m/z (ES⁺) = 478.21 [M + H]⁺ (Method A) |
| 152 | 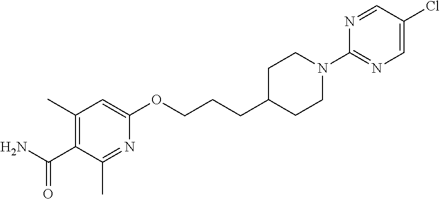 | 6-{3-[1-(5-Chloro- pyrimidin-2-yl)piperidin-4- yl]propoxy}-2,4-dimethyl- nicotinamide | RT = 3.72 min; m/z (ES⁺) = 404.17 [M + H]⁺ (Method A) |
| 153 | 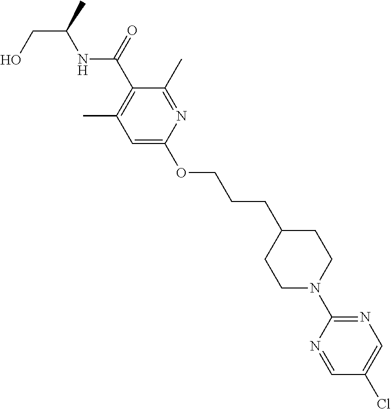 | 6-{3-[1-(5-Chloro- pyrimidin-2-yl)piperidin-4- yl]propoxy}-N-((R)-2- hydroxy-1-methylethyl)-2,4- dimethylnicotinamide | RT = 3.63 min; m/z (ES⁺) = 462.19 [M + H]⁺ (Method A) |
| 154 | 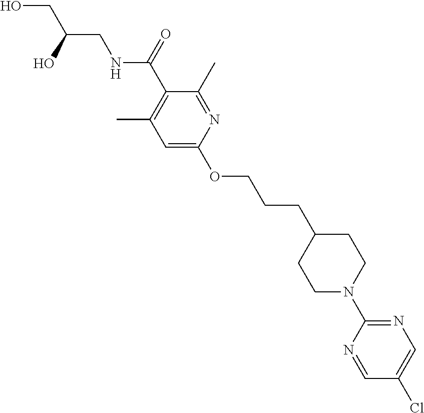 | 6-{3-[1-(5-Chloro- pyrimidin-2-yl)piperidin-4- yl]propoxy}-N-((R)-2,3- dihydroxypropyl)-2,4- dimethylnicotinamide | RT = 3.35 min; m/z (ES⁺) = 478.21 [M + H]⁺ (Method A) |
| 155 | 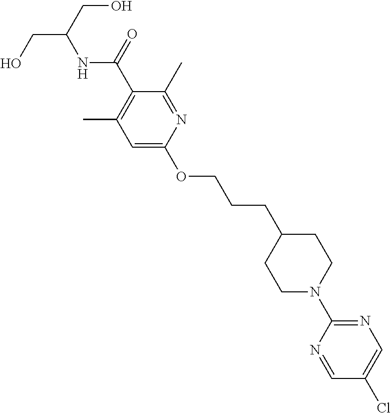 | 6-{3-[1-(5-Chloro- pyrimidin-2-yl)piperidin-4- yl]propoxy}-N-(2-hydroxy- 1-hydroxymethylethyl)-2,4- dimethylnicotinamide | RT = 3.30 min; m/z (ES⁺) = 478.19 [M + H]⁺ (Method A) |
| 156 | 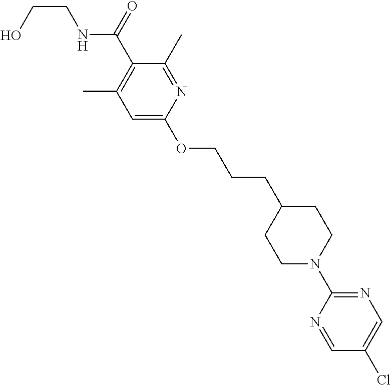 | 6-{3-[1-(5-Chloro- pyrimidin-2-yl)piperidin-4- yl]propoxy}-N-(2-hydroxy- ethyl)-2,4-dimethyl- nicotinamide | RT = 3.52 min; m/z (ES⁺) = 448.19 [M + H]⁺ (Method A) |
| 157 | 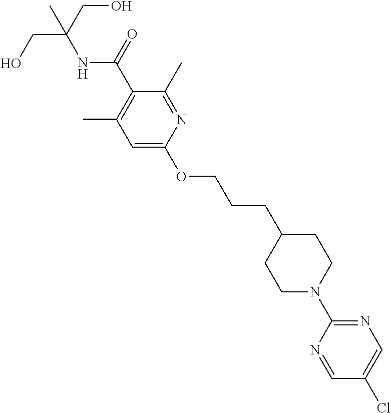 | 6-{3-[1-(5-Chloro- pyrimidin-2-yl)piperidin-4- yl]propoxy}-N-(2-hydroxy- 1-hydroxymethyl-1-methyl- ethyl)-2,4-dimethyl- nicotinamide | RT = 3.65 min; m/z (ES⁺) = 492.22 [M + H]⁺ (Method A) |

The amino-containing amides listed in Table 11 were synthesised by a two-step process: (i) 6-{3-[1-(5-chloropyrimidin-2-yl)piperidin-4-yl]propoxy}-2,4-dimethylnicotinic acid (Preparation 59) was condensed with the appropriate Boc-amino-containing amine, employing procedures similar to that outlined in Example 145; (ii) the Boc group was removed with 4M HCl in dioxane, utilizing a protocol similar to that outlined in Preparation 50.

TABLE 11

| Ex | Structure | Name | Spectra: LCMS |
|---|---|---|---|
| 158 | 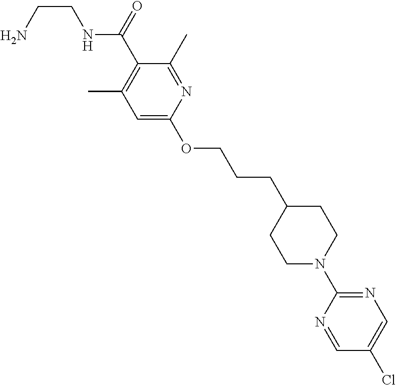 | N-(2-Aminoethyl)-6-{3-[1- (5-chloropyrimidin-2-yl)- piperidin-4-yl]propoxy}-2,4- dimethylnicotinamide | RT = 2.77 min; m/z (ES⁺) = 447.21 [M + H]⁺ (Method A) |
| 159 | 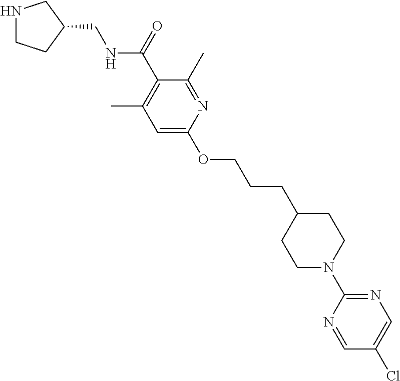 | 6-{3-[1-(5-Chloro- pyrimidin-2-yl)piperidin-4- yl]propoxy}-2,4-dimethyl- N-(R)-1-pyrrolidin-3- ylmethylnicotinamide | RT = 2.77 min; m/z (ES⁺) = 487.23 [M + H]⁺ (Method A) |

### Example 160

### N—((S)-2,3-Dihydroxypropyl)-6-{3-[1-(5-ethylpyrimidin-2-yl)piperidin-4-yl]-propoxy}-2-methylnicotinamide

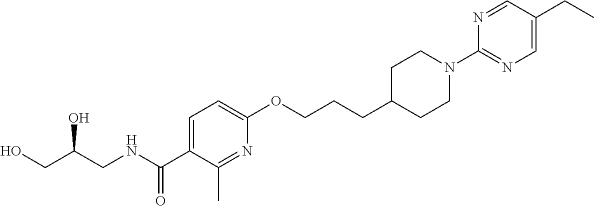

The title compound was synthesized from N—((S)-2,3-dihydroxypropyl)-2-methyl-6-(3-piperidin-4-ylpropoxy)nicotinamide (Preparation 51, 341 mg, 881 μmol) and 2-chloro-5-ethylpyrimidine (189 mg, 1.32 mmol) employing a procedure similar to that outlined in Example 147: RT=3.08 min; ink (ES⁺)=458.38 [M+M]⁺ (Method A).

### Example 161

### 5′-Fluoro-4-[3-(5-methanesulfonyl-6-methylpyridin-2-yloxy)propyl]-3,4,5,6-tetrahydro-2H-[1,2′]bipyridinyl

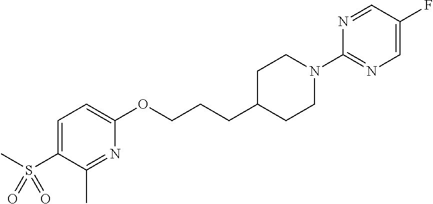

2,5-Difluoropyridine (65.0 mg, 490 lima) and DBU (72.0 μL, 490 μmol) were added to a solution of 3-methanesulfonyl-2-methyl-6-(3-piperidin-4-yl-propoxy)pyridine hydrochloride (Preparation 54, 125 mg, 330 μmol) in DMSO (500 μL) and the resulting solution stirred at 50° C. for 72 h. The reaction mixture was poured into H₂O (50 mL), extracted with EtOAc (2×75 mL) and the combined organic extracts washed with brine, dried (MgSO₄), filtered and concentrated in vacuo. Purification by column chromatography (IH-EtOAc; 1:1) afforded the title compound: RT=3.67 min; m/z (ES⁺)=408.11 [M+H]⁺ (Method A).

### Example 162

### 5-Fluoro-2-{4-[3-(5-methanesulfonyl-6-methylpyridin-2-yloxy)propyl]piperidin-1-yl}pyrimidine

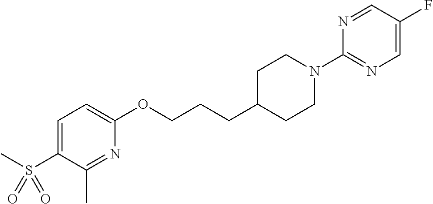

The title compound was synthesized from 3-methanesulfonyl-2-methyl-6-(3-piperidin-4-ylpropoxypyridine hydrochloride (Preparation 54, 125 mg, 330 μmol) and 2-chloro-5-fluoropyrimidine (65.0 mg, 490 mmol) employing a procedure similar to that outlined in Example 161: RT=4.24 min; m/z (ES⁺)=409.12 [M+H]⁺ (Method A).

The following compound may be prepared by methods analogous to those described above:

### 2-{4-[(R)-3-(4-Methanesulfonylphenoxy)-1-methylpropyl]piperidin-1-yl}-5-trifluoromethylpyrimidine

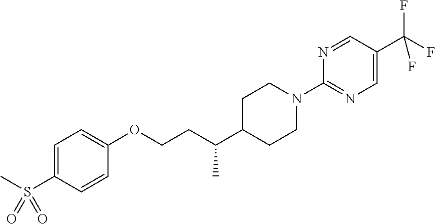

The biological activity of the compounds of the invention may be tested in the following assay systems:

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Ste3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di(β-D-galactopyranoside) was used (FDG), a substrate for the enzyme that releases fluorescein, allowing a fluorimetric read-out. 20 μl per well of 500 μM FDG/2.5% Triton X100 was added (the detergent was necessary to render the cells permeable). After incubation of the cells with the substrate for 60 min, 20 μl per well of 1M sodium carbonate was added to terminate the reaction and enhance the fluorescent signal. The plates were then read in a fluorimeter at 485/535 nm.

The compounds of the invention give an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line may be used to investigate the effect of compounds of the invention on intracellular levels of cyclic AMP (cAMP). The cell monolayers are washed with phosphate buffered saline and stimulated at 37° C. for 30 min with various concentrations of compound in stimulation buffer plus 1% DMSO. Cells are then lysed and cAMP content determined using the Perkin Elmer AlphaScreen™ (Amplified Luminescent Proximity Homogeneous Assay) cAMP kit. Buffers and assay conditions are as described in the manufacturer's protocol.

### In Vivo Feeding Study

The effect of compounds of the invention on body weight and food and water intake may be examined in freely-feeding male Sprague-Dawley rats maintained on reverse-phase lighting. Test compounds and reference compounds are dosed by appropriate routes of administration (e.g. intraperitoneally or orally) and measurements made over the following 24 h. Rats are individually housed in polypropylene cages with metal grid floors at a temperature of 21±4° C. and 55±20% humidity. Polypropylene trays with cage pads are placed beneath each cage to detect any food spillage. Animals are maintained on a reverse phase light-dark cycle (lights off for 8 h from 09.30-17.30 h) during which time the room was illuminated by red light. Animals have free access to a standard powdered rat diet and tap water during a two week acclimatization period. The diet is contained in glass feeding jars with aluminum lids. Each lid had a 3-4 cm hole in it to allow access to the food. Animals, feeding jars and water bottles are weighed (to the nearest 0.1 g) at the onset of the dark period. The feeding jars and water bottles are subsequently measured 1, 2, 4, 6 and 24 h after animals are dosed with a compound of the invention and any significant differences between the treatment groups at baseline compared to vehicle-treated controls.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38 (1):44-8).

cAMP Assay

HIT-T15 cells were plated in standard culture medium in 96-well plates at 100,000 cells/0.1 ml/well and cultured for 24 hr and the medium was then discarded. Cells were incubated for 15 min at room temperature with 100 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 ul lysis buffer (5 mM HEPES, 0.3% Tween-20, 0.1% BSA, pH 7.4) was added per well and the plate was shaken at 900 rpm for 20 min Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μl reactions were set up containing 8 μl sample, 5 μl acceptor bead mix and 12 μl detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells were plated in standard culture medium in 12-well plates at 106 cells/1 ml/well and cultured for 3 days and the medium was then discarded. Cells were washed×2 with supplemented Krebs-Ringer buffer (KRB) containing 119 mM NaCl, 4.74 mM KCl, 2.54 mM CaCl₂, 1.19 mM MgSO₄, 1.19 mM KH2PO4, 25 mM NaHCO₃, 10 mM HEPES at pH 7.4 and 0.1% bovine serum albumin. Cells were incubated with 1 ml KRB at 37° C. for 30 min which was then discarded. This was followed by a second incubation with KRB for 30 min, which was collected and used to measure basal insulin secretion levels for each well. Compound dilutions (0, 0.1, 0.3, 1, 3, 10 uM) were then added to duplicate wells in 1 ml KRB, supplemented with 5.6 mM glucose. After 30 min incubation at 37° C. samples were removed for determination of insulin levels. Measurement of insulin was done using the Mercodia Rat insulin ELISA kit, following the manufacturers instructions, with a standard curve of known insulin concentrations. For each well insulin levels were corrected by subtraction of the basal secretion level from the pre-incubation in the absence of glucose. Data was analysed using XLfit 3 software.

Representative compounds of the invention were found to increase insulin secretion at an EC₅₀ of less than 10 μM. Compounds showing an EC₅₀ of less than 1 μM in the insulin secretion assay may be preferred.

### Oral Glucose Tolerance Tests

The effects of compounds of the invention on oral glucose (Glc) tolerance were evaluated in male Sprague-Dawley rats. Food was withdrawn 16 h before administration of Glc and remained withdrawn throughout the study. Rats had free access to water during the study. A cut was made to the animals' tails, then blood (1 drop) was removed for measurement of basal Glc levels 60 min before administration of the Glc load. Then, the rats were weighed and dosed orally with test compound or vehicle (20% aqueous hydroxypropyl-β-cyclodextrin) 45 min before the removal of an additional blood sample and treatment with the Glc load (2 g kg⁻¹ p.o.). Blood samples were then taken from the cut tip of the tail 5, 15, 30, 60, 120, and 180 min after Glc administration. Blood glucose levels were measured just after collection using a commercially available glucose-meter (OneTouch® Ultra™ from Lifescan). Representative compounds of the invention statistically reduced the Glc excursion at doses of ≦10 mg kg⁻¹.

The effects of compounds of the invention on oral glucose (Glc) tolerance may also evaluated in male C57B1/6 or male ob/ob mice. Food is withdrawn 5 h before administration of Glc and remained withdrawn throughout the study. Mice have free access to water during the study. A cut is made to the animals' tails, then blood (20 μL) is removed for measurement of basal Glc levels 45 min before administration of the Glc load. Then, the mice are weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) are then taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood are then added to 180 μL of Trinders glucose reagent (Sigma enzymatic (Trinder) colorimetric method) in a 96-well assay plate. After mixing, the samples are left at rt for 30 min before being read against Glc standards (Sigma glucose/urea nitrogen combined standard set).

1. A compound of formula (I), or a pharmaceutically acceptable salt thereof: 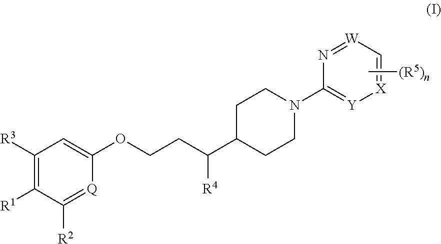
wherein Q is CH or N;
one of W, X and Y is N or CH and the others are CH where the H may be replaced by R⁵ when present;
R¹ is —SO₂Me or —CONHR⁶;
R², R³ and R⁴ are independently selected from hydrogen and methyl;
n is 0, 1 or 2;
R⁵ is independently C₁₋₄ alkyl, C₁₋₄ alkoxy, fluoro, chloro, C₁₋₃ fluoroalkyl or benzyl;
R⁶ is hydrogen, 3-azetidinyl, 3-pyrrolidinyl, 3-pipendinyl, or 4-piperidinyl, wherein the azetidinyl, pyrrolidinyl and piperidinyl rings may be optionally substituted with OH, CH₂OH or CH₃, C₁₋₃ alkyl, C₂₋₄ alkyl substituted by —N(R⁷)₂ and/or one or two hydroxy groups, or C₁₋₄ alkyl substituted by a 4- to 6-membered nitrogen-containing heterocyclic ring; and
R⁷ is independently hydrogen or methyl.

2. A compound according to claim 1, or a pharmaceutically acceptable salt thereof, wherein Q is CH.

3. A compound according to claim 1, or a pharmaceutically acceptable salt thereof, wherein Q is N.

4. A compound according to claim 1, or a pharmaceutically acceptable salt thereof, wherein W and X are CH.

5. A compound according to claim 4, or a pharmaceutically acceptable salt thereof, wherein W, X and Y are CH.

6. A compound according to claim 1, or a pharmaceutically acceptable salt thereof, wherein R¹ is —SO₂Me.

7. A compound according to claim 1, or a pharmaceutically acceptable salt thereof, wherein R¹ is —CONHR⁶.

8. A compound according to claim 1, or a pharmaceutically acceptable salt thereof, wherein one or both of R² and R³ are methyl.

9. A compound according to claim 1, or a pharmaceutically acceptable salt thereof, wherein n is 1.

10. A compound according to claim 9, or a pharmaceutically acceptable salt thereof, wherein R⁵ is meta or para to the point of attachment to the piperidinyl nitrogen.

11. A compound according to claim 10, or a pharmaceutically acceptable salt thereof, wherein R⁵ is para to the point of attachment to the piperidinyl nitrogen.

12. A compound according to claim 1, or a pharmaceutically acceptable salt thereof, wherein R⁴ is hydrogen.

13. A compound according to claim 1, or a pharmaceutically acceptable salt thereof, wherein R⁴ is methyl.

14. A compound according to claim 13, or a pharmaceutically acceptable salt thereof, wherein R⁴ is methyl and the stereocenter produced has the (R)-configuration.

15. A compound according to claim 1, or a pharmaceutically acceptable salt thereof, wherein R⁵ is C₁₋₃ alkyl, fluoro, chloro or C₁₋₃ fluoroalkyl.

16. A compound according to claim 1, or a pharmaceutically acceptable salt thereof, wherein R⁶ is hydrogen or C₂₋₃ alkyl substituted by —N(R⁷)₂ or one or two hydroxy groups.

17. A compound according to claim 16, or a pharmaceutically acceptable salt thereof, wherein R⁶ is 2-hydroxyethyl, 2-hydroxy-1-methylethyl, 2,3-dihydroxypropyl or 2-hydroxy-1-hydroxy methylethyl.

18. A compound of claim 1, wherein the compound is any one of Examples 1 to 162, or a pharmaceutically acceptable salt thereof.

19. A pharmaceutical composition comprising a compound according to claim 1, or a pharmaceutically acceptable salt thereof and a pharmaceutically acceptable carrier.

20. A method for the treatment of a disease or condition in which GPR119 plays a role comprising a step of administering to a subject in need thereof an effective amount of a compound according to claim 1, or a pharmaceutically acceptable salt thereof.

21. A method for the regulation of satiety comprising a step of administering to a subject in need thereof an effective amount of a compound according to claim 1, or a pharmaceutically acceptable salt thereof.

22-27. (canceled)

28. The method of claim 20, wherein the disease or condition in which GPR119 plays a role is obesity, diabetes, metabolic syndrome (syndrome X), impaired glucose tolerance, hyperlipidemia, hypertriglyceridemia, hypercholesterolemia, low HDL levels, or hypertension.
